(19) United States
(12) Patent Application Publication
(10) Pub. No.: US 2012/0252792 A1
(43) Pub. Date: Oct. 4, 2012

(54) METHODS AND COMPOSITIONS FOR MODULATING RHO-MEDIATED GENE TRANSCRIPTION

(75) Inventors: Richard Neubig, Ann Arbor, MI (US); Chris Evelyn, Wheatley Heights, NY (US); Jenny Ryu, Ann Arbor, MI (US); Scott Larsen, Ann Arbor, MI (US); Jessica Bell, Ann Arbor, MI (US)

(73) Assignee: THE REGENTS OF THE UNIVERSITY OF MICHIGAN, Ann Arbor, MI (US)

(21) Appl. No.: 13/496,827

(22) PCT Filed: Sep. 17, 2010
(86) PCT No.: PCT/US10/49312

§ 371 Date: Jun. 13, 2012

Related U.S. Application Data
(60) Provisional application No. 61/243,370, filed on Sep. 17, 2009.

(51) Int. Cl. A61K 31/166; A61P 35/00; A61P 29/00; A61P 15/10; A61P 9/00; A61P 19/02; A61P 43/00; A61P 19/08; A61P 19/10; A61P 35/04; C12N 9/96; C07C 237/22; A61K 31/167; C07C 237/52; C07D 209/08; A61K 31/404; C07D 277/30; A61K 31/426; C07D 243/08; C07D 261/04; A61K 31/42; A61K 31/551; C07C 233/78

(52) U.S. Cl. 514/218; 564/185; 514/617; 435/188; 564/164; 514/620; 564/155; 514/616; 548/491; 514/415; 548/204; 514/365; 540/492; 548/240; 514/378

(57) ABSTRACT

The invention provides methods, compositions, and kits for the inhibition of members of the Rho GTPase family. Specifically, the invention provides methods, compositions and kits for the inhibition of RhoA and/or RhoC transcriptional signalling. The invention finds use in treatment of Rho-mediated disease states (e.g., tumor metastasis, inflammation, inflammatory disease), Rho-mediated biological conditions, and in cell signaling research.

### CROSS REFERENCE TO RELATED APPLICATIONS

This application claims priority to U.S. Provisional Patent Application Ser. No. 61/243,370, filed Sep. 17, 2009, hereby incorporated by reference in its entirety.

This invention was made with government support under contract numbers GM39561, CA069568 and F31 CA113268 awarded by the National Institutes of Health. The government has certain rights in the invention.

### FIELD OF THE INVENTION

The invention relates to methods, compositions, and kits for the inhibition of members of the Rho GTPase family. Specifically, the invention relates to methods, compositions and kits for the inhibition of RhoA and/or RhoC transcriptional signaling. The invention finds use in treatment of Rho-mediated disease states (e.g., tumor metastasis), Rho-mediated biological conditions, and in cell signaling research.

### BACKGROUND OF THE INVENTION

Cancer metastasis is a significant medical problem in the United States, where it is estimated that >500,000 cancer-related deaths in 2003 resulted from metastatic tumors rather than primary tumors (approximately 90% of cancer deaths). Cancer metastasis requires malfunction in several tightly regulated cellular processes controlling cell movement from a primary site to a secondary site. These cellular processes include cell survival, adhesion, migration, and proteolysis resulting in extracellular matrix remodeling, immune escape, angiogenesis and lymphangiogenesis, and target ‘homing’. Most existing cancer treatments focus on killing tumor cells; however, such chemotherapeutic intervention leads to substantial toxicity to healthy cells and tissue. Since spread, or metastasis, of cancers is the primary cause of cancer-related mortalities, there is urgent need for agents that specifically inhibit or prevent signals that trigger metastasis.

Rho proteins are overexpressed in various tumors, including colon, breast, lung, testicular germ cell, and head and neck squamous-cell carcinoma (Sawyer, Expert Opin. Investig. Drugs., 13: 1-9, 2004; herein incorporated by reference in its entirety). The rho family of small GTP binding proteins plays important roles in many normal biological processes and in cancer (Schmidt and Hall, Genes Dev., 16:1587-1609, 2002; Burridge and Wennerberg, Cell, 116:167-179, 2004; each herein incorporated by reference in its entirety). This family includes three main groups: rho, rac, and cdc42. Rho is activated by numerous external stimuli including growth factor receptors, immune receptors, cell adhesion, and G protein coupled receptors (GPCRs) (Schmidt and Hall, Genes Dev., 16:1587-1609, 2002, Sah et al., Annu. Rev. Pharmacol. Toxicol., 40:459-489, 2000; each herein incorporated by reference in its entirety).

RhoA and rhoC play roles in metastasis (Clark et al., Nature 406:532-535, 2000; Ikoma et al., Clin Cancer Res 10:1192-1200, 2004; Shikada et al., Clin Cancer Res 9:5282-5286, 2003; Wu et al., Breast Cancer Res Treat 84:3-12, 2004; Hakem et al, Genes Dev 19:1974-9, 2005; each herein incorporated by reference in its entirety). Both rhoA and rac1 can regulate the function of the extracellular matrix (ECM) proteins, ezrin, moesin, and radixin, by the phosphorylation of ezrin via the rhoA pathway and the phosphorylation of the ezrin antagonist, neurofibromatosis 2, by the racl pathway (Shaw et al., Dev Cell 1:63-72, 2001; Matsui et al., J Cell Biol 140:647-657, 1998; each herein incorporated by reference in its entirety). These ECM proteins promote cell movement by utilizing the ECM receptor, CD44, to link the actin cytoskeleton with the plasma membrane. In addition, rhoA and racl regulate ECM remodeling by controlling the levels of matrix metalloproteinases (MMPs) or their antagonists, tissue inhibitors of metalloproteinases (TIMPs) (Bartolome et al., Cancer Res 64:2534-2543, 2004; herein incorporated by reference in its entirety). RhoA is also required for monocyte tail retraction during transendothelial migration, indicating a role in extravasation, which is a key process in metastasis (Worthylake et al., J Cell Biol 154:147-160, 2001; herein incorporated by reference in its entirety).

In addition to cytoskeletal effects, rhoA and rhoC induce gene transcription via the serum response factor, SRF. SRF is associated with cellular transformation and epithelial-mesenchymal transformation (Iwahara et al., Oncogene 22:5946-5957, 2003; Psichari et al., J Biol Chem 277:29490-29495, 2002; each herein incorporated by reference in its entirety). Rho activates SRF via release of the transcriptional coactivator, megakaryoblastic leukemia protein (MKL) (Cen et al., Mol Cell Biol 23:6597-6608, 2003; Miralles et al., Cell 113:329-342, 2003; Selvaraj and Prywes, J Biol Chem 278:41977-41987, 2003; each herein incorporated by reference in its entirety). MKL, like the rhoGEF LARG, was first identified as a site of gene translocation in leukemia (megakaryoblastic leukemia) (Mercher et al., Genes Chromosomes Cancer 33:22-28, 2002; herein incorporated by reference in its entirety). The protein product of the translocated MKL gene is hyperactive compared to the wild-type protein. MKL has also been called modified in acute leukemia (MAL) or BSAC (Miralles et al., Cell 113:329-342, 2003; Sasazuki et al., J Biol Chem 277:28853-28860, 2002; each herein incorporated by reference in its entirety). Interestingly, MKL/MAL/BSAC was identified in an antiapoptosis screen for genes that abrogate tumor necrosis factor-induced cell death (Sasazuki et al., J Biol Chem 277:28853-28860, 2002; herein incorporated by reference in its entirety). As a consequence of rho signaling, MKL translocates to the nucleus and binds SRF leading to the expression of c-fos which, along with c-jun, forms the transcription factor AP-1. The AP-1 transcription factor promotes the activity of various MMPs and other cell motility genes (Benbow and Brinckerhoff, Matrix Biol 15:519-526, 1997; herein incorporated by reference in its entirety). Expression of these genes leads to cancer cell invasion and metastasis. Thus, there is a link between rho-controlled biological processes and cancer metastasis. Similarly, both LARG and MKL are important players in these processes.

The relative contributions of rho and rac proteins in the metastatic phenotype has been studied (Sahai and Marshall, Nat Rev Cancer 2:133-142, 2002; Whitehead et al., Oncogene 20:1547-1555, 2001; each herein incorporated by reference in its entirety). Sahai and Marshall (Nat Cell Biol 5:711-719, 2003; herein incorporated by reference in its entirety) showed that different tumor cell lines exhibit different mechanisms of motility and invasion. In particular, 375m2 melanoma and LS174T colon carcinoma cell lines showed striking “rounded” and “blebbed” morphology during invasion into Matrigel matrices. This invasion was entirely rho-dependent and was blocked by C3 exotoxin, the N17rho dominant negative protein, and a ROCK kinase inhibitor. In contrast, two other cell lines were blocked instead by a rac dominant negative mutation, but not rho or ROCK inhibitors. These latter two cell lines (BE colon carcinoma and SW962 squamous cell carcinoma) had elongated morphologies. A third line showed a mixed morphology and was blocked partially by both rho and rac inhibitors. Additionally, mice lacking rhoC have greatly reduced metastasis of virally-induced breast tumors to lung (Hakem et al, Genes Dev 19:1974-9, 2005; herein incorporated by reference in its entirety). Also, knock-down of SRF or its transcriptional co-activator MKL reduced lung metastases from breast or melanoma xenografts (Medjkane et al, Nat Cell Biol. 11:257-68, 2009; herein incorporated in its entirety). Clearly there is important heterogeneity in mechanisms of tumor cell behavior that contributes to metastasis. It is widely recognized that cell growth and apoptosis mechanisms vary greatly among tumors, necessitating customized therapeutic approaches.

Development of targeted therapies for cancer requires an understanding of critical molecular steps in specific tumors. Signaling by growth factors, G protein coupled receptors, and small GTP binding proteins are known to play important roles in the biology of cancer. In some tumors, rho GTPases are involved in cell transformation and metastasis. In particular, RhoA and RhoC are upregulated in melanoma and in breast, lung, prostate, and pancreatic cancer. Expression of RhoA and/or RhoC has been correlated with tumor aggressiveness and invasiveness. In addition, thrombin, lysophosphatidic acid (LPA), and other agonists at G protein coupled receptors within the rho pathway are associated with changes in cell motility, invasion, and metastatic behavior.

A key principle in targeted therapies is that only those tumors utilizing a particular signaling pathway are susceptible to such therapy. The design and identification of novel chemical inhibitors of rho pathways contributes to the armamentarium of targeted cancer therapies. An increase in such targeted cancer therapies is urgently needed. In addition, improved therapeutic agents for other rho-mediated diseases processes are needed. Other rho-mediated disease states include but are not limited to pulmonary arterial hypertension (Naeije et al., Expert Opinin. Pharmacother. 8:2247-2265, 2007; herein incorporated by reference in its entirety); axon regeneration following nerve damage due to spinal cord injury, brain injury, and neurodegenerative diseases (Gross et al., Cell Transpl. 16:245-262, 2007; herein incorporated by reference in its entirety), Raynaud's phenomenon (Flavahan, Rheum. Dis. Clin. North Am. 34:81, 2007; herein incorporated by reference in its entirety), cerebral vascular disease (Chrissobolis et al., Stroke 37:2174-2180, 2006; herein incorporated by reference in its entirety), cardiovascular disease (Noma et al., Am. J. Physiol. Cell Physiol. 290(3):C661-8, 2006; herein incorporated by reference in its entirety), and erectile dysfunction (Jin et al., Clin. Sci. (Lond.) 110:153-165, 2006; herein incorporated by reference in its entirety).

What is needed are new compositions and methods for targeted therapy to assist in the treatment and management of cancer.

### SUMMARY OF THE INVENTION

Certain embodiments of the present invention relate to methods and compositions for the inhibition of members of the Rho GTPase family. Specifically, some embodiments of the present invention relate to methods and compositions for the inhibition of RhoA and/or RhoC signal transduction. Some embodiments of the present invention find use in treatment of Rho-mediated disease states (e.g., tumor metastasis, inflammatory diseases), Rho-mediated biological conditions (e.g., inflammation), and cell signaling research.

In some embodiments of the present invention, identification of rho-inhibiting agents was facilitated by use of a dual luciferase assay. In this system, an SRE.L-firefly luciferase reporter allowed specific detection of Rho/MLK1 pathway activity expressed in a cell line (e.g., PC-3 prostate cancer cell line) in which this pathway had been up-regulated by transient transfection with the Gα12QL activator of Rho/MLK1. The assay system further comprised co-transfection of cells with a thymidine kinase-Renilla luciferase (TK-Renilla) reporter to discern agents causing general transcriptional inhibition, rather than Rho-specific inhibition. To assess the impact of tested agents on cell viability, WST1-metabolism assays were used to determine whether acute nonspecific toxic effects were caused by the candidate agents. This screening allowed the identification of novel, nontoxic compositions that specifically inhibit the Rho/MLK1 pathway.

In some embodiments, the present invention is directed to compositions and methods for the treatment of disease states, disorders, and biological conditions that involve rho-signaling. More specifically, the present invention is directed to compositions for inhibiting rho-mediated gene transcription, where the compositions comprise an isolated compound that has any of the following general structural formulas such as:

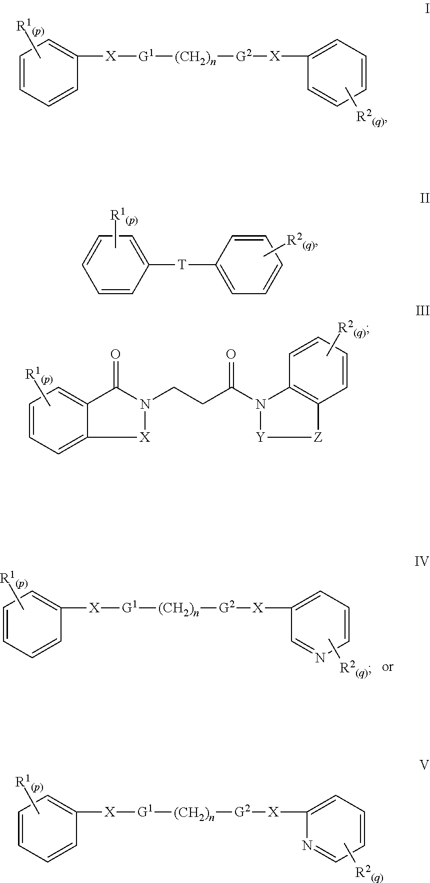

wherein G¹ and G² may be independently selected groups such as (C═O)NH, NH(C═O), (C═O)NHO, NH, O, NH(C═O)O, O(C═O)NH, and heteroaryl; and
wherein p and q may be independently 1, 2, 3, 4, or 5; and
wherein X, Y, Z are independently: (CH₂)ₘ wherein m is 0, 1, or 2 and n is 1, 2, or 3; and
wherein heteroaryl is selected from the group consisting of 1,3-thiazole and isoxazole; and
wherein T is selected from the group consisting of:

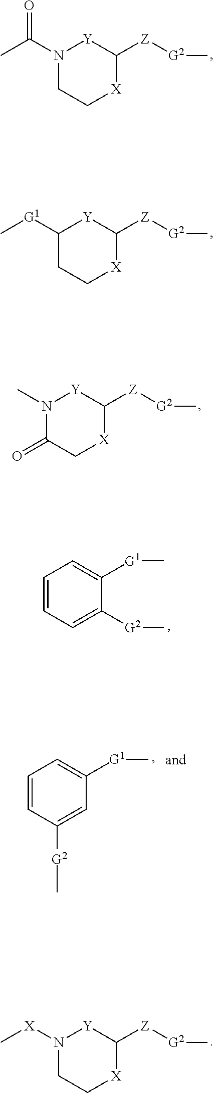

In some embodiments, G¹ and G² are not CONH when R¹ is 3,5-bis(CF₃) and R² is 4-Cl.

In some embodiments, R¹ and R² may be one or more functional groups independently selected from the group consisting of halogen, CF₃, OCF₃, CN, O(C1-C6 alkyl), and C1-C 6 alkyl.

In some embodiments, R¹ and R² may be one or more independently selected functional groups such as halogen; CF₃; OCF₃; CN; O(C1-C6 alkyl); C1-C6 alkyl; hydrogen; alkyl; substituted alkyl; OH; a chemical moiety comprising an aryl subgroup; a chemical moiety comprising a substituted aryl subgroup; a chemical moiety comprising a cycloaliphatic subgroup; a chemical moiety comprising a substituted cycloaliphatic subgroup; a chemical moiety comprising a heterocyclic subgroup; a chemical moiety comprising a substituted heterocyclic subgroup; a chemical moiety comprising at least one ester subgroup; a chemical moiety comprising at least one ether subgroup; a linear or branched, saturated or unsaturated, substituted or non-substituted, aliphatic chain having at least 2 carbons; a chemical moiety comprising sulfur; a chemical moiety comprising nitrogen; —OR—, wherein R comprises one or more of a chemical moiety comprising an aryl subgroup; a chemical moiety comprising a substituted aryl subgroup; a chemical moiety comprising a cycloaliphatic subgroup; a chemical moiety comprising a substituted cycloaliphatic subgroup; a chemical moiety comprising a heterocyclic subgroup; a chemical moiety comprising a substituted heterocyclic subgroup; a linear or branched, saturated or unsaturated, substituted or non-substituted, aliphatic chain having at least 2 carbons; a chemical moiety comprising at least one ester subgroup; a chemical moiety comprising at least one ether subgroup; a chemical moiety comprising sulfur; a chemical moiety comprising nitrogen.

In some embodiments, the rho-inhibiting agent is a composition such as

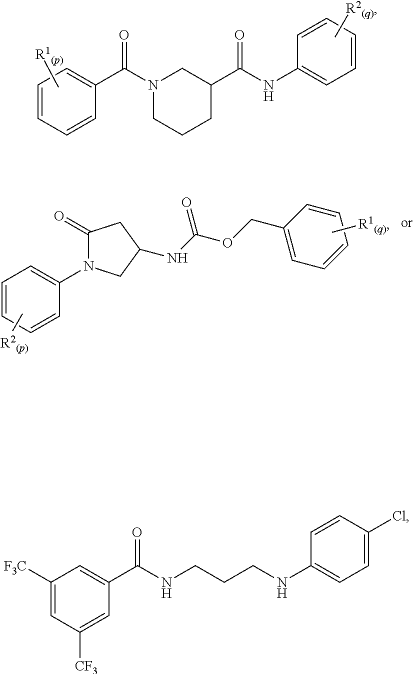

wherein p and q may be independently 1, 2, 3, 4, or 5.

It should be understood that derivatives of the above compounds, as well as pure enantiomers (pure R, pure S), or racemic mixture, salts, esters, and prodrugs are contemplated within the compositions and methods of embodiments of the present invention. In some embodiments, the composition is in a pharmaceutically appropriate formulation for administration to a human subject. In some embodiments, the composition has an IC₅₀ value for rho protein of between 1 and 50,000 nM (e.g., 1-10; 10-25; 25-50; 50-100; 100-1,000; 1,000-5,000; 5,000-50,000 nM). In some embodiments, the rho protein is a protein such as rhoA or rhoC. In some embodiments, the composition results in 50% (e.g., 40%, 30%, etc.) or less inhibition of WST-1 metabolism in an in vitro cell when the composition is administered to the in vitro cell at a concentration of 10 μM.

In certain embodiments, the present invention provides a method of treating or preventing a rho-mediated disease in a subject comprising administering a composition in a pharmaceutically appropriate formulation to the subject, wherein the composition has a structure as described above or elsewhere herein.

In some embodiments, the rho-mediated disease is a disease or disease state such as cancer, inflammation, inflammatory disease, pulmonary arterial hypertension, axon regeneration following nerve damage, Raynaud's phenomenon, cerebral vascular disease, cardiovascular disease, or erectile dysfunction. In some embodiments, the cancer type is a type such as fibrosarcoma, myxosarcoma, liposarcoma, chondrosarcoma, osteogenic sarcoma, chordoma, angiosarcoma, endotheliosarcoma, lymphangiosarcoma, Ewing's tumor, lymphangioendotheliosarcoma, synovioma, mesothelioma, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, polycythemia vera, lymphoma, multiple myeloma, Waldenstrom's macroglobulinemia, or heavy chain disease. In some embodiments, the inflammatory disease is a disease such as arthritis, rheumatoid arthritis, psoriatic arthritis, osteoarthritis, degenerative arthritis, polymyalgia rheumatic, ankylosing spondylitis, reactive arthritis, gout, pseudogout, inflammatory joint disease, systemic lupus erythematosus, polymyositis, and fibromyalgia. Additional types of arthritis include achilles tendinitis, achondroplasia, acromegalic arthropathy, adhesive capsulitis, adult onset Still's disease, anserine bursitis, avascular necrosis, Behcet's syndrome, bicipital tendinitis, Blount's disease, brucellar spondylitis, bursitis, calcaneal bursitis, calcium pyrophosphate deposition disease (CPPD), crystal deposition disease, Caplan's syndrome, carpal tunnel syndrome, chondrocalcinosis, chondromalacia patellae, chronic synovitis, chronic recurrent multifocal osteomyelitis, Churg-Strauss syndrome, Cogan's syndrome, corticosteroid-induced osteoporosis, costosternal syndrome, CREST syndrome, cryoglobulinemia, degenerative joint disease, dermatomyositis, diabetic finger sclerosis, diffuse idiopathic skeletal hyperostosis (DISH), discitis, discoid lupus erythematosus, drug-induced lupus, Duchenne's muscular dystrophy, Dupuytren's contracture, Ehlers-Danlos syndrome, enteropathic arthritis, epicondylitis, erosive inflammatory osteoarthritis, exercise-induced compartment syndrome, Fabry's disease, familial Mediterranean fever, Farber's lipogranulomatosis, Felty's syndrome, Fifth's disease, flat feet, foreign body synovitis, Freiberg's disease, fungal arthritis, Gaucher's disease, giant cell arteritis, gonococcal arthritis, Goodpasture's syndrome, granulomatous arteritis, hemarthrosis, hemochromatosis, Henoch-Schonlein purpura, Hepatitis B surface antigen disease, hip dysplasia, Hurler syndrome, hypermobility syndrome, hypersensitivity vasculitis, hypertrophic osteoarthropathy, immune complex disease, impingement syndrome, Jaccoud's arthropathy, juvenile ankylosing spondylitis, juvenile dermatomyositis, juvenile rheumatoid arthritis, Kawasaki disease, Kienbock's disease, Legg-Calve-Perthes disease, Lesch-Nyhan syndrome, linear scleroderma, lipoid dermatoarthritis, Lofgren's syndrome, Lyme disease, malignant synovioma, Marfan's syndrome, medial plica syndrome, metastatic carcinomatous arthritis, mixed connective tissue disease (MCTD), mixed cryoglobulinemia, mucopolysaccharidosis, multicentric reticulohistiocytosis, multiple epiphyseal dysplasia, mycoplasmal arthritis, myofascial pain syndrome, neonatal lupus, neuropathic arthropathy, nodular panniculitis, ochronosis, olecranon bursitis, Osgood-Schlatter's disease, osteoarthritis, osteochondromatosis, osteogenesis imperfecta, osteomalacia, osteomyelitis, osteonecrosis, osteoporosis, overlap syndrome, pachydermoperiostosis Paget's disease of bone, palindromic rheumatism, patellofemoral pain syndrome, Pellegrini-Stieda syndrome, pigmented villonodular synovitis, piriformis syndrome, plantar fasciitis, polyarteritis nodos, Polymyalgia rheumatic, polymyositis, popliteal cysts, posterior tibial tendinitis, Pott's disease, prepatellar bursitis, prosthetic joint infection, pseudoxanthoma elasticum, psoriatic arthritis, Raynaud's phenomenon, reactive arthritis/Reiter's syndrome, reflex sympathetic dystrophy syndrome, relapsing polychondritis, retrocalcaneal bursitis, rheumatic fever, rheumatoid vasculitis, rotator cuff tendinitis, sacroiliitis, salmonella osteomyelitis, sarcoidosis, saturnine gout, Scheuermann's osteochondritis, scleroderma, septic arthritis, seronegative arthritis, shigella arthritis, shoulder-hand syndrome, sickle cell arthropathy, Sjogren's syndrome, slipped capital femoral epiphysis, spinal stenosis, spondylolysis, staphylococcus arthritis, Stickler syndrome, subacute cutaneous lupus, Sweet's syndrome, Sydenham's chorea, syphilitic arthritis, systemic lupus erythematosus (SLE), Takayasu's arteritis, tarsal tunnel syndrome, tennis elbow, Tietse's syndrome, transient osteoporosis, traumatic arthritis, trochanteric bursitis, tuberculosis arthritis, arthritis of Ulcerative colitis, undifferentiated connective tissue syndrome (UCTS), urticarial vasculitis, viral arthritis, Wegener's granulomatosis, Whipple's disease, Wilson's disease, or yersinial arthritis. In some embodiments, the method further comprises administering an agent such as a chemotherapeutic agent or an anti-inflammatory agent.

In certain embodiments, the present invention provides a method of reducing metastatic spread of a cancer cell in a subject comprising administering a compound in a pharmaceutically appropriate formulation to the subject wherein the compound has a structure as described above or elsewhere herein.

In certain embodiments, the present invention provides method of reducing growth of a cancer cell in a subject comprising a compound in a pharmaceutically appropriate formulation to the subject wherein the compound has a structure as described above or elsewhere herein.

In certain embodiments, the present invention provides a method of inhibiting the in vitro activity of rho protein comprising exposing the rho protein to a compound, wherein the compound has a structure as described above or elsewhere herein.

The methods of embodiments of the present invention may employ any of the compounds described herein, including those above and those found in the Examples section, as well as derivatives thereof.

In some embodiments, the in vitro rho protein is a protein such as rhoA and rhoC. In some embodiments, the activity is assessed by measuring the expression of a rho-mediated gene. In some embodiments, the rho-mediated gene is an endogenous gene. In some embodiments, the rho-mediated gene is an exogenous (e.g., reporter) gene. In some embodiments, the measurement comprises assessing the level of a rho-mediated gene transcript. In some embodiments, the measurement comprises assessing the level of a rho-mediated protein. In some embodiments, the measurement comprises assessing the level of activity of a rho-mediated protein.

Additional embodiments will be apparent to persons skilled in the relevant art based on the teachings contained herein.

### DESCRIPTION OF THE DRAWINGS

FIG. 1 shows a characterization of the SRE.L system in HEK293T cells. Gα13 activates the rhoGEF LARG, which activates rho. This leads to actin polymerization through mDia1 and ROCK. On actin polymerization, the coactivator MKL1 is released from actin and translocates into the nucleus. There, MLK1 interacts with the transcription factor SRF and the complex activates the SRE(ΔTCF) response element, leading to luciferase expression.

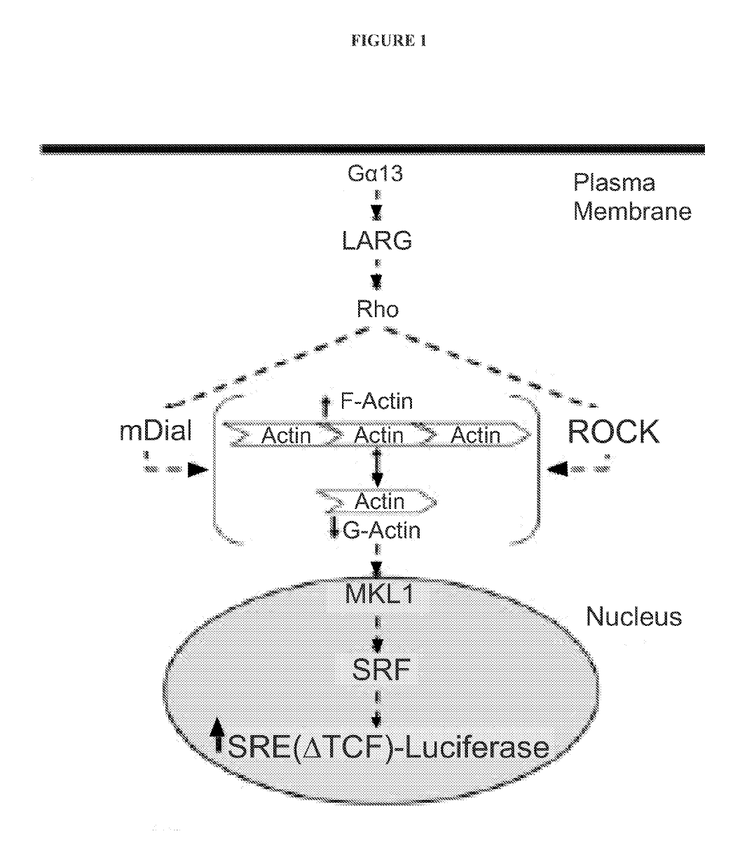

FIG. 2 shows that CCG-1423 inhibits cancer cell proliferation and survival. A, PC-3 cells were treated for 27 h with 100 μmol/L LPA in the presence or absence of various concentrations of CCG-1423, labeled with BrdUrd, and stained, and absorbance was read 450 nm. B, various cell lines were treated with 30 mmol/L LPA with or without 0.3 mmol/L CCG-1423, and then on day 8, WST-1 absorbance was read at 450 nm Black columns indicate four melanoma lines with differing expression of RhoC (A375M2 and SK-MeI-147 have high expression, whereas the parental line A375 (used to derive A375M2) and SK-Mel-28 have lower expression); gray columns indicate several other cancer cell lines; white column indicates the nontransformed fibroblast line WI-38. C, A375 and A375M2 cells were treated with 3 μmol/L CCG-1423 or 3 μmol/L daunorubicin for 25 h, and then caspase-3 activity was measured with a fluorescent substrate (Z0DEVD0R110) using excitation at 485 nm and emission detection at 520 nm. In A and C, data are expressed as a percentage of the no FBS control. In B, data are expressed as percentage of the LPA+DMSO control. All data represent n=3.

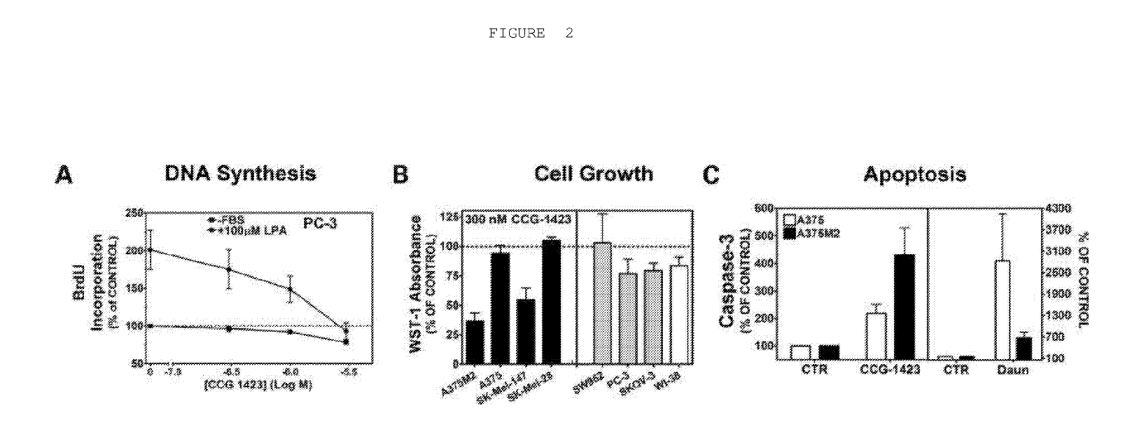

FIG. 3 shows that CCG-1423 inhibits prostate cancer cell invasion. A, LPA stimulates invasion of SKOV-3 but not PC-3 cells. Invasion of Matrigel®-coated filters by serum-starved PC-3 prostate cancer or SKOV-3 ovarian cancer cells were measured with or without 30 μmol/L LPA as chemoattractant. B, CCG-1423 inhibits PC-3 cell invasion, whereas PTX inhibits SKOV-3 cell invasion. The effects of CCG-1423 (3 μmol/L) and PTX (100 ng/mL) on spontaneous (PC-3) or LPA-stimulated (SKOV-3) invasion through Matrigel® were measured. In B, data are expressed as a percentage of control. All data represent n=3.

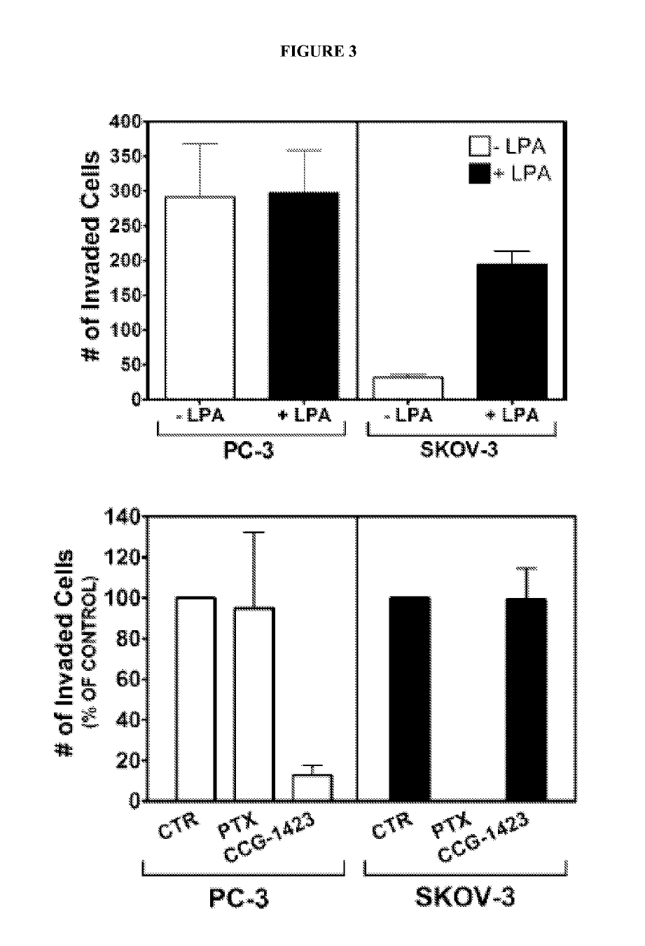

### DEFINITIONS

To facilitate an understanding of the present invention, a number of terms and phrases are defined below:

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “subject suspected of having cancer” refers to a subject that presents one or more symptoms indicative of a cancer (e.g., a noticeable lump or mass) or is being screened for a cancer (e.g., during a routine physical). A subject suspected of having cancer may also have one or more risk factors. A subject suspected of having cancer has generally not been tested for cancer. However, a “subject suspected of having cancer” encompasses an individual who has received a preliminary diagnosis (e.g., a CT scan showing a mass) but for whom a confirmatory test (e.g., biopsy and/or histology) has not been done or for whom the stage of cancer is not known. The term further includes people who once had cancer (e.g., an individual in remission). A “subject suspected of having cancer” is sometimes diagnosed with cancer and is sometimes found to not have cancer.

As used herein, the term “subject diagnosed with a cancer” refers to a subject who has been tested and found to have cancerous cells. The cancer may be diagnosed using any suitable method, including but not limited to, biopsy, x-ray, blood test, and the diagnostic methods of the present invention. A “preliminary diagnosis” is one based only on visual (e.g., CT scan or the presence of a lump) and/or molecular screening tests.

As used herein, the term “initial diagnosis” refers to a test result of initial cancer diagnosis that reveals the presence or absence of cancerous cells (e.g., using a biopsy and histology).

As used herein, the term “post surgical tumor tissue” refers to cancerous tissue that has been removed from a subject (e.g., during surgery).

As used herein, the term “identifying the risk of said tumor metastasizing” refers to the relative risk (e.g., the percent chance or a relative score) of a tumor metastasizing.

As used herein, the term “identifying the risk of said tumor recurring” refers to the relative risk (e.g., the percent chance or a relative score) of a tumor recurring in the same organ as the original tumor.

As used herein, the term “subject at risk for cancer” refers to a subject with one or more risk factors for developing a specific cancer. Risk factors include, but are not limited to, gender, age, genetic predisposition, environmental exposure, and previous incidents of cancer, preexisting non-cancer diseases, and lifestyle.

As used herein, the term “characterizing cancer in subject” refers to the identification of one or more properties of a cancer sample in a subject, including but not limited to, the presence of benign, pre-cancerous or cancerous tissue and the stage of the cancer.

As used herein, the term “stage of cancer” refers to a qualitative or quantitative assessment of the level of advancement of a cancer. Criteria used to determine the stage of a cancer include, but are not limited to, the size of the tumor, whether the tumor has spread to other parts of the body and where the cancer has spread (e.g., within the same organ or region of the body or to another organ).

Staging of cancer can also be based on the revised criteria of TNM staging by the American Joint Committee for Cancer (AJCC) published in 1988. Staging is the process of describing the extent to which cancer has spread from the site of its origin. It is used to assess a patient's prognosis and to determine the choice of therapy. The stage of a cancer is determined by the size and location in the body of the primary tumor, and whether it has spread to other areas of the body. Staging involves using the letters T, N and M to assess tumors by the size of the primary tumor (T); the degree to which regional lymph nodes (N) are involved; and the absence or presence of distant metastases (M)—cancer that has spread from the original (primary) tumor to distant organs or distant lymph nodes. Each of these categories is further classified with a number 1 through 4 to give the total stage. Once the T, N and M are determined, a “stage” of I, II, III or IV is assigned. Stage I cancers are small, localized and usually curable. Stage II and III cancers typically are locally advanced and/or have spread to local lymph nodes. Stage IV cancers usually are metastatic (have spread to distant parts of the body) and generally are considered inoperable.

As used herein, the term “characterizing tissue in a subject” refers to the identification of one or more properties of a tissue sample (e.g., including but not limited to, the presence of cancerous tissue, the presence of pre-cancerous tissue that is likely to become cancerous, and the presence of cancerous tissue that is likely to metastasize).

As used herein, the term “providing a prognosis” refers to providing information regarding the impact of the presence of cancer (e.g., as determined by the diagnostic methods of the present invention) on a subject's future health (e.g., expected morbidity or mortality, the likelihood of getting cancer, and the risk of metastasis).

As used herein, the term “non-human animals” refers to all non-human animals including, but not limited to, vertebrates such as rodents, non-human primates, ovines, bovines, ruminants, lagomorphs, porcines, caprines, equines, canines, felines, ayes, etc.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used herein, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., cancer). Test compounds comprise both known and potential therapeutic compounds. A test compound can be determined to be therapeutic by screening using the screening methods of the present invention.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, and industrial samples. Such examples are not however to be construed as limiting the sample types applicable to the present invention.

As used herein, the terms “rho” or “rho proteins” refer to the narrowly defined rho subfamily that includes rhoA, rhoB, rhoC, etc. and is described in (Sahai and Marshall, Nat Rev Cancer 2:133-142, 2002; herein incorporated by reference in its entirety). These terms do not refer to the larger rho family (i.e. do not refer to rac and cdc42). The term “Rho family” is used to designate the larger group including the three rho subfamilies (rho, rac, and cdc42).

As used herein, the term “effective amount” refers to the amount of a compound (e.g., a rho-inhibiting compound having a structure presented above or elsewhere described herein) sufficient to effect beneficial or desired results. An effective amount can be administered in one or more administrations, applications or dosages and is not limited to or intended to be limited to a particular formulation or administration route.

As used herein, the term “co-administration” refers to the administration of at least two agent(s) (e.g., a rho-inhibiting compound having a structure presented above or elsewhere described herein) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

As used herein, the term “pharmaceutical composition” refers to the combination of an active agent with a carrier, inert or active, making the composition especially suitable for diagnostic or therapeutic use in vivo, in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants. (See e.g., Martin, Remington's Pharmaceutical Sciences, 15th Ed., Mack Publ. Co., Easton, Pa. [1975]).

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NWₐ⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

As used herein, the term “instructions for administering said compound to a subject,” and grammatical equivalents thereof, includes instructions for using the compositions contained in a kit for the treatment of conditions characterized by viral infection (e.g., providing dosing, route of administration, decision trees for treating physicians for correlating patient-specific characteristics with therapeutic courses of action). The rho-inhibiting compounds of the present invention (e.g. as shown in structures above and elsewhere presented herein) can be packaged into a kit, which may include instructions for administering the compounds to a subject.

As used herein, the term “chemical moiety” refers to any chemical compound containing at least one carbon atom. Examples of chemical moieties include, but are not limited to, aromatic chemical moieties, chemical moieties comprising sulfur, chemical moieties comprising nitrogen, hydrophilic chemical moieties, and hydrophobic chemical moieties.

As used herein, the term “heteroaryl” refers to an aromatic ring with at least one carbon replaced by O, S or N.

As used herein, the term “aliphatic” represents the groups including, but not limited to, alkyl, alkenyl, alkynyl, alicyclic.

As used herein, the term “aryl” represents a single aromatic ring such as a phenyl ring, or two or more aromatic rings (e.g., bisphenyl, naphthalene, anthracene), or an aromatic ring and one or more non-aromatic rings. The aryl group can be optionally substituted with a lower aliphatic group (e.g., alkyl, alkenyl, alkynyl, or alicyclic). Additionally, the aliphatic and aryl groups can be further substituted by one or more functional groups including, but not limited to, —NH₂, —NHCOCH₃, —OH, lower alkoxy (C₁-C₄), halo (—F, —Cl, —Br, or —I).

As used herein, the term “substituted aliphatic,” refers to an alkane, alkene, alkyne, or alicyclic moiety where at least one of the aliphatic hydrogen atoms has been replaced by, for example, a halogen, an amino, a hydroxy, a nitro, a thio, a ketone, an aldehyde, an ester, an amide, a lower aliphatic, a substituted lower aliphatic, or a ring (aryl, substituted aryl, cycloaliphatic, or substituted cycloaliphatic, etc.). Examples of such include, but are not limited to, 1-chloroethyl and the like.

As used herein, the term “substituted aryl” refers to an aromatic ring or fused aromatic ring system consisting of at least one aromatic ring, and where at least one of the hydrogen atoms on a ring carbon has been replaced by, for example, a halogen, an amino, a hydroxy, a nitro, a thio, a ketone, an aldehyde, an ester, an amide, a lower aliphatic, a substituted lower aliphatic, or a ring (aryl, substituted aryl, cycloaliphatic, or substituted cycloaliphatic). Examples of such include, but are not limited to, hydroxyphenyl and the like.

As used herein, the term “cycloaliphatic” refers to an aliphatic structure containing a fused ring system. Examples of such include, but are not limited to, decalin and the like.

As used herein, the term “substituted cycloaliphatic” refers to a cycloaliphatic structure where at least one of the aliphatic hydrogen atoms has been replaced by a halogen, a nitro, a thio, an amino, a hydroxy, a ketone, an aldehyde, an ester, an amide, a lower aliphatic, a substituted lower aliphatic, or a ring (aryl, substituted aryl, cycloaliphatic, or substituted cycloaliphatic). Examples of such include, but are not limited to, 1-chlorodecalyl, bicyclo-heptanes, octanes, and nonanes (e.g., nonrbornyl) and the like.

As used herein, the term “heterocyclic” represents, for example, an aromatic or nonaromatic ring containing one or more heteroatoms. The heteroatoms can be the same or different from each other. Examples of heteratoms include, but are not limited to nitrogen, oxygen and sulfur. Aromatic and nonaromatic heterocyclic rings are well-known in the art. Some nonlimiting examples of aromatic heterocyclic rings include pyridine, pyrimidine, indole, purine, quinoline and isoquinoline. Nonlimiting examples of nonaromatic heterocyclic compounds include piperidine, piperazine, morpholine, pyrrolidine and pyrazolidine. Examples of oxygen containing heterocyclic rings include, but not limited to furan, oxirane, 2H-pyran, 4H-pyran, 2H-chromene, and benzofuran. Examples of sulfur-containing heterocyclic rings include, but are not limited to, thiophene, benzothiophene, and parathiazine. Examples of nitrogen containing rings include, but not limited to, pyrrole, pyrrolidine, pyrazole, pyrazolidine, imidazole, imidazoline, imidazolidine, pyridine, piperidine, pyrazine, piperazine, pyrimidine, indole, purine, benzimidazole, quinoline, isoquinoline, triazole, and triazine. Examples of heterocyclic rings containing two different heteroatoms include, but are not limited to, phenothiazine, morpholine, parathiazine, oxazine, oxazole, thiazine, and thiazole. The heterocyclic ring is optionally further substituted with one or more groups selected from aliphatic, nitro, acetyl (i.e., —C(═O)—CH₃), or aryl groups.

As used herein, the term “substituted heterocyclic” refers to a heterocylic structure where at least one of the ring carbon atoms is replaced by oxygen, nitrogen, phosphorous, or sulfur, and where at least one of the aliphatic hydrogen atoms has been replaced by a halogen, hydroxy, a thio, nitro, an amino, a ketone, an aldehyde, an ester, an amide, a lower aliphatic, a substituted lower aliphatic, or a ring (aryl, substituted aryl, cycloaliphatic, or substituted cycloaliphatic). Examples of such include, but are not limited to 2-chloropyranyl.

As used herein, the term “a chemical moiety that participates in hydrogen bonding” represents a group that can accept or donate a proton to form a hydrogen bond thereby. Some specific non-limiting examples of moieties that participate in hydrogen bonding include fluoro-containing groups, oxygen-containing groups, sulfur-containing groups, and nitrogen-containing groups that are well-known in the art (e.g., a hydroxyl group, a phenol group, an amide group, a sulfonamide group, an amine group, an aniline group, a benzimidizalone group, a carbamate group, and an imidizole group). Some examples of oxygen-containing groups that participate in hydrogen bonding include: hydroxy, lower alkoxy, lower carbonyl, lower carboxyl, lower ethers and phenolic groups. The qualifier “lower” as used herein refers to lower aliphatic groups (C₁-C₄) to which the respective oxygen-containing functional group is attached. Thus, for example, the term “lower carbonyl” refers to inter alia, formaldehyde, acetaldehyde. Some nonlimiting examples of nitrogen-containing groups that participate in hydrogen bond formation include amino and amido groups. Additionally, groups containing both an oxygen and a nitrogen atom can also participate in hydrogen bond formation. Examples of such groups include nitro, N-hydroxy and nitrous groups. It is also possible that the hydrogen-bond acceptor in the present invention can be the π electrons of an aromatic ring.

As used herein, the term “derivative” of a compound refers to a chemically modified compound wherein the chemical modification takes place at a functional group of the compound (e.g., aromatic ring). Such derivatives include, but are not limited to, esters of alcohol-containing compounds, esters of carboxy-containing compounds, amides of amine-containing compounds, amides of carboxy-containing compounds, imines of amino-containing compounds, acetals of aldehyde-containing compounds, ketals of carbonyl-containing compounds, and the like.

As used herein, the term “toxic” refers to any detrimental or harmful effects on a cell or tissue as compared to the same cell or tissue prior to the administration of the toxicant.

### DETAILED DESCRIPTION OF THE INVENTION

Activation of signaling systems such as tyrosine kinase receptors, G protein coupled receptors (Gα12/13), and integrins leads to stimulation of Rho-dependent cellular processes. RhoA and RhoC in particular have been shown to be critical for tumor metastasis. Rho mediates cytoskeletal rearrangements hut also induces gene transcription via MKL1, a serum response factor coactivator and oncogene. The contribution of gene transcriptional effects to Rho's role in cancer metastasis is of interest to those skilled in the art of cancer biology. Utilizing a Rho/MKL1-specific SRE.L-luciferase reporter assay, Evelyn et al (Mol Canc. Ther 6:2249-60, 2007; herein incorporated by reference in its entirety) described a potent, small molecule inhibitor of Rho/MKL1 mediated transcription, CCG-1423. This compound has an IC₅₀ of 1 μM for blocking gene transcription and also for inhibiting DNA synthesis, cell growth and survival, and Matrigel® invasion by Rho—expressing prostate cancer and melanoma cell lines. In vivo studies using CCG-1423 were limited by non-specific toxicity. Therefore, in experiments conducted during the development of some embodiments of the present invention, potent and less toxic inhibitors of the Rho transcription pathway were sought and discovered. Such rho-inhibiting compounds find use as improved chemical tools and as therapeutics.

In experiments conducted during the development of some embodiments of the present invention, the Rho/MKL1-specific SRE.L-luciferase reporter assay was used in conjunction with a thymidine kinase Renilla reporter as a measure of non specific transcriptional effects (e.g., Example 1). A number of compounds were identified that show improved selectivity for SRE.L and minimal TK promoter inhibition. Some composition embodiments of the present invention have IC₅₀ values between 4 μM and 20 μM and show reduced toxicity in vitro based on a WST1 (MTT-like) cell viability readout (e.g., Example 1). In addition to their utility for therapeutic purposes, composition embodiments of the present invention find use as research tools that are more selective and potent inhibitors of Rho/MKL1-mediated gene transcription for use in mechanistic studies and in vivo studies of Rho/MKL1 transcriptional inhibition.

### A. Rho Cell Signaling Pathway

The mechanism of signaling by heterotrimeric G protein-coupled receptors that activate rho has been described (Sah et al., Annu Rev Pharmacol Toxicol 40:459-489, 2000; herein incorporated by reference in its entirety). The discovery of a family of unique rho guanine nucleotide exchange factors (rhoGEFs), p115rhoGEF (Hart et al., J Biol Chem 271:25452-25458, 1996; herein incorporated by reference in its entirety), PDZrhoGEF (Fukuhara et al., J Biol Chem 274:5868-5879, 1999; herein incorporated by reference in its entirety), and LARG (Leukemia-associated rhoGEF) (Kourlas et al., Proc Natl Acad Sci U S A 97:2145-2150, 2000; herein incorporated by reference in its entirety) suggested a common mechanism. They contain a regulator of G protein signaling (RGS) domain that binds activated Gα12 (Suzuki et al., Proc Natl Acad Sci USA 100:733-738, 2003; herein incorporated by reference in its entirety) and Gα13 (Hart et al., Science 280:2112-2114, 1998; herein incorporated by reference in its entirety) causing rhoGEF activation. Thus, the RGS-rhoGEFs appear to serve as effectors of activated Gα12/13 and as molecular bridges between the heterotrimeric G protein alpha subunits and rho. This is a novel action of an RGS-domain containing protein, since they typically inhibit GPCR responses (Neubig and Siderovski, Nat Rev Drug Discov 1:187-197, 2002; herein incorporated by reference in its entirety). A role for RGS-rhoGEF proteins in cellular rho signaling by GPCRs, such as those for thrombin and lysophosphatidic acid (LPA), has been shown by studies with dominant negative constructs (Mao et al., Proc Natl Acad Sci USA 95:12973-12976, 1998; Majumdar et al., J Biol Chem 274:26815-26821, 1999; each herein incorporated by reference in its entirety) and inhibition of signaling by expression of the RGS-domains which act as Gα12/13 inhibitors (Fukuhara et al., FEBS Lett 485:183-188, 2000; herein incorporated by reference in its entirety).

Direct evidence that these RGS-RhoGEF proteins mediate GPCR signals and information about which rhoGEF(s) are downstream of which receptors has been shown (Wang et al., J Biol. Chem., 279(28):28831-28834, 2004; herein incorporated by reference in its entirety). Experimentally, rho activation is detected directly by measurements of GTP-bound active rho precipitated from cell lysates with effector fusion proteins such as GST-rhotekin (Reid et al., J Biol Chem 271:13556-13560, 1996; herein incorporated by reference in its entirety) or indirectly by any number of functional readouts. The 1321N1 astrocytoma cell system is a well-studied model of thrombin-induced rho activation (Majumdar et al., J Biol Chem 273:10099-10106, 1998; herein incorporated by reference in its entirety). Thrombin induces both cell rounding and enhanced cell proliferation in these astrocytoma cells by mechanisms that are independent of known second messengers but are blocked by rho inhibitors.

Wang et al. (J Biol. Chem., 279(28):28831-28834, 2004; herein incorporated by reference in its entirety) used HEK293T cells and an aggressive, metastatic, human prostate cancer cell line, PC-3, to test the role of the three RGS-rhoGEFs (LARG, p115-, and PDZrhoGEF) in receptor signaling. HEK293 and PC-3 cells express all three of these proteins. Transcriptional expression of PDZrhoGEF and LARG exceeds that of p115. PC-3 cells over-express the thrombin receptor (PAR1) and have an increased propensity to metastasize to bone compared to lines that have lower PAR1 expression (Cooper et al., Cancer 97:739-747, 2003; herein incorporated by reference in its entirety). To demonstrate a role for rhoGEFs in GPCR signaling and to define the specificity of their actions, it was shown by siRNA targeting that LARG mediates thrombin responses while the LPA response is mediated by PDZrhoGEF. This was the first direct demonstration of a role for an RGS-rhoGEF in G protein coupled receptor signaling. Furthermore, it pinpointed critical RGS-rhoGEFs (LARG and PDZrhoGEF) and allowed use of rhoGEFs in screening for modulators of rho-stimulated activities.

Development of synthetic RNAi molecules against the three members of this protein family showed that in PC-3 cells, the thrombin receptor (PAR1) utilized LARG while the LPA receptor utilized PDZ-rhoGEF for inducing cell rounding (Wang et al., J Biol. Chem., 279(28):28831-28834, 2004; herein incorporated by reference in its entirety). In addition, direct measurements of thrombin-induced rho activation in HEK293T cells by GST-rhotekin pulldown also demonstrated a dependence on LARG. In the course of these studies, the rho transcription reporter method that uses the rho-specific SRE.L Luciferase was developed. This transcriptional reporter method, described in detail therein, was used for screening a small chemical library for possible rho inhibitors (Evelyn et al., Mol. Canc. Ther. 6:2249-60, 2007; herein incorporated by reference in its entirety). Additional inhibitors were identified as described below.

### B. Method of Treatment or Prevention of Cancer, Inflammation, Inflammatory Diseases, and Other Disorders

In some embodiments of the present invention, methods and compositions are provided for the treatment of tumors in cancer therapy. It is contemplated that such therapy can be employed in the treatment of any cancer for which a specific signature has been identified or which can be targeted. Cell proliferative disorders, or cancers, contemplated to be treatable with the methods of the present invention include human sarcomas and carcinomas, including, but not limited to, fibrosarcoma, myxosarcoma, liposarcoma, chondrosarcoma, osteogenic sarcoma, chordoma, angiosarcoma, endotheliosarcoma, lymphangiosarcoma, Ewing's tumor, lymphangioendotheliosarcoma, synovioma, mesothelioma, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemias, acute lymphocytic leukemia and acute myelocytic leukemia (myeloblastic, promyelocytic, myelomonocytic, monocytic and erythroleukemia), chronic leukemia (chronic myelocytic (granulocytic) leukemia and chronic lymphocytic leukemia), polycythemia vera, lymphoma (Hodgkin's disease and non-Hodgkin's disease), multiple myeloma, Waldenstrom's macroglobulinemia, and heavy chain disease.

In some embodiments of the present invention, methods and compositions are provided for the treatment of inflammatory diseases or inflammatory responses. Inflammation may occur, for example, in response to infection (e.g., infection by a pathogenic organism), wounding, cell damage, or irritants. Inflammatory diseases include but are not limited to arthritis, rheumatoid arthritis, psoriatic arthritis, osteoarthritis, degenerative arthritis, polymyalgia rheumatic, ankylosing spondylitis, reactive arthritis, gout, pseudogout, inflammatory joint disease, systemic lupus erythematosus, polymyositis, and fibromyalgia. Additional types of arthritis include achilles tendinitis, achondroplasia, acromegalic arthropathy, adhesive capsulitis, adult onset Still's disease, anserine bursitis, avascular necrosis, Behcet's syndrome, bicipital tendinitis, Blount's disease, brucellar spondylitis, bursitis, calcaneal bursitis, calcium pyrophosphate deposition disease (CPPD), crystal deposition disease, Caplan's syndrome, carpal tunnel syndrome, chondrocalcinosis, chondromalacia patellae, chronic synovitis, chronic recurrent multifocal osteomyelitis, Churg-Strauss syndrome, Cogan's syndrome, corticosteroid-induced osteoporosis, costosternal syndrome, CREST syndrome, cryoglobulinemia, degenerative joint disease, dermatomyositis, diabetic finger sclerosis, diffuse idiopathic skeletal hyperostosis (DISH), discitis, discoid lupus erythematosus, drug-induced lupus, Duchenne's muscular dystrophy, Dupuytren's contracture, Ehlers-Danlos syndrome, enteropathic arthritis, epicondylitis, erosive inflammatory osteoarthritis, exercise-induced compartment syndrome, Fabry's disease, familial Mediterranean fever, Farber's lipogranulomatosis, Felty's syndrome, Fifth's disease, flat feet, foreign body synovitis, Freiberg's disease, fungal arthritis, Gaucher's disease, giant cell arteritis, gonococcal arthritis, Goodpasture's syndrome, granulomatous arteritis, hemarthrosis, hemochromatosis, Henoch-Schonlein purpura, Hepatitis B surface antigen disease, hip dysplasia, Hurler syndrome, hypermobility syndrome, hypersensitivity vasculitis, hypertrophic osteoarthropathy, immune complex disease, impingement syndrome, Jaccoud's arthropathy, juvenile ankylosing spondylitis, juvenile dermatomyositis, juvenile rheumatoid arthritis, Kawasaki disease, Kienbock's disease, Legg-Calve-Perthes disease, Lesch-Nyhan syndrome, linear scleroderma, lipoid dermatoarthritis, Lofgren's syndrome, Lyme disease, malignant synovioma, Marfan's syndrome, medial plica syndrome, metastatic carcinomatous arthritis, mixed connective tissue disease (MCTD), mixed cryoglobulinemia, mucopolysaccharidosis, multicentric reticulohistiocytosis, multiple epiphyseal dysplasia, mycoplasmal arthritis, myofascial pain syndrome, neonatal lupus, neuropathic arthropathy, nodular panniculitis, ochronosis, olecranon bursitis, Osgood-Schlatter's disease, osteoarthritis, osteochondromatosis, osteogenesis imperfecta, osteomalacia, osteomyelitis, osteonecrosis, osteoporosis, overlap syndrome, pachydermoperiostosis Paget's disease of bone, palindromic rheumatism, patellofemoral pain syndrome, Pellegrini-Stieda syndrome, pigmented villonodular synovitis, piriformis syndrome, plantar fasciitis, polyarteritis nodos, Polymyalgia rheumatic, polymyositis, popliteal cysts, posterior tibial tendinitis, Pott's disease, prepatellar bursitis, prosthetic joint infection, pseudoxanthoma elasticum, psoriatic arthritis, Raynaud's phenomenon, reactive arthritis/Reiter's syndrome, reflex sympathetic dystrophy syndrome, relapsing polychondritis, retrocalcaneal bursitis, rheumatic fever, rheumatoid vasculitis, rotator cuff tendinitis, sacroiliitis, salmonella osteomyelitis, sarcoidosis, saturnine gout, Scheuermann's osteochondritis, scleroderma, septic arthritis, seronegative arthritis, shigella arthritis, shoulder-hand syndrome, sickle cell arthropathy, Sjogren's syndrome, slipped capital femoral epiphysis, spinal stenosis, spondylolysis, staphylococcus arthritis, Stickler syndrome, subacute cutaneous lupus, Sweet's syndrome, Sydenham's chorea, syphilitic arthritis, systemic lupus erythematosus (SLE), Takayasu's arteritis, tarsal tunnel syndrome, tennis elbow, Tietse's syndrome, transient osteoporosis, traumatic arthritis, trochanteric bursitis, tuberculosis arthritis, arthritis of Ulcerative colitis, undifferentiated connective tissue syndrome (UCTS), urticarial vasculitis, viral arthritis, Wegener's granulomatosis, Whipple's disease, Wilson's disease, and yersinial arthritis.

Additional rho-mediated disease states for which compositions and methods of the present invention are appropriate include but are not limited to pulmonary arterial hypertension (Naeije et al., Expert Opinin. Pharmacother. 8:2247-2265, 2007; herein incorporated by reference in its entirety); axon regeneration following nerve damage due to spinal cord injury, brain injury, and neurodegenerative diseases (Gross et al., Cell Transpl. 16:245-262, 2007; herein incorporated by reference in its entirety), Raynaud's phenomenon (Flavahan, Rheum. Dis. Clin. North Am. 34:81, 2007; herein incorporated by reference in its entirety), cerebral vascular disease (Chrissobolis et al., Stroke 37:2174-2180, 2006; herein incorporated by reference in its entirety), cardiovascular disease (Noma et al., Am. J. Physiol. Cell Physiol. 290(3):C661-8, 2006; herein incorporated by reference in its entirety), and erectile dysfunction (Jin et al., Clin. Sci. (Lond.) 110:153-165, 2006; herein incorporated by reference in its entirety).

### C. Pharmaceutical Formulations

Where clinical applications are contemplated, in some embodiments of the present invention, the rho-inhibiting compositions of the present invention are prepared as part of a pharmaceutical composition in a form appropriate for the intended application. Generally, this entails preparing compositions that are essentially free of pyrogens, as well as other impurities that could be harmful to humans or animals. However, in some embodiments of the present invention, a straight rho-inhibiting composition formulation may be administered using one or more of the routes described herein.

In preferred embodiments, the rho-inhibiting compositions of the present invention are used in conjunction with appropriate salts and buffers to render delivery of the compositions in a stable manner to allow for uptake by target cells. Buffers also are employed when the rho-inhibiting compositions are introduced into a patient. Aqueous compositions comprise an effective amount of the rho-inhibiting composition to cells dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. Except insofar as any conventional media or agent is incompatible with the vectors or cells of the present invention, its use in therapeutic compositions is contemplated. Supplementary active ingredients may also be incorporated into the compositions.

In some embodiments of the present invention, the active compositions include classic pharmaceutical preparations. Administration of these compositions according to the present invention is via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection.

The active rho-inhibiting compositions of the present invention may also be administered parenterally or intraperitoneally or intratumorally. Solutions of the active compounds as free base or pharmacologically acceptable salts are prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it may be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, rho-inhibiting compositions of the present invention are administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution is suitably buffered, if necessary, and the liquid diluent first rendered isotonic with sufficient saline or glucose. These particular aqueous solutions are especially suitable for intravenous, intramuscular, subcutaneous and intraperitoneal administration. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). In some embodiments of the present invention, the active particles or agents are formulated within a therapeutic mixture to comprise about 0.0001 to 1.0 milligrams, or about 0.001 to 0.1 milligrams, or about 0.1 to 1.0 or even about 10 milligrams per dose or so. Multiple doses may be administered.

Additional formulations that are suitable for other modes of administration include vaginal suppositories and pessaries. A rectal pessary or suppository may also be used. Suppositories are solid dosage forms of various weights and shapes, usually medicated, for insertion into the rectum, vagina or the urethra. After insertion, suppositories soften, melt or dissolve in the cavity fluids. In general, for suppositories, traditional binders and carriers may include, for example, polyalkylene glycols or triglycerides; such suppositories may be formed from mixtures containing the active ingredient in the range of 0.5% to 10%, preferably 1%-2%. Vaginal suppositories or pessaries are usually globular or oviform and weighing about 5 g each. Vaginal medications are available in a variety of physical forms, e.g., creams, gels or liquids, which depart from the classical concept of suppositories. In addition, suppositories may be used in connection with colon cancer. The rho-inhibiting compositions of the present invention also may be formulated as inhalants for the treatment of lung cancer and such like.

### D. Dosage

“Treating” within the context of the instant invention, means an alleviation, in whole or in part, of symptoms associated with a disorder or disease, or slowing, inhibiting or halting of further progression or worsening of those symptoms, or prevention or prophylaxis of the disease or disorder in a subject at risk for developing the disease or disorder. Thus, e.g., treating metastatic cancer may include inhibiting or preventing the metastasis of the cancer, a reduction in the speed and/or number of the metastasis, a reduction in tumor volume of the metastasized cancer, a complete or partial remission of the metastasized cancer or any other therapeutic benefit. As used herein, a “therapeutically effective amount” of a compound of the invention refers to an amount of the compound that alleviates, in whole or in part, symptoms associated with a disorder or disease, or slows, inhibits or halts further progression or worsening of those symptoms, or prevents or provides prophylaxis for the disease or disorder in a subject at risk for developing the disease or disorder.

A subject is any animal that can benefit from the administration of a compound as described herein. In some embodiments, the subject is a mammal, for example, a human, a primate, a dog, a cat, a horse, a cow, a pig, a rodent, such as for example a rat or mouse. Typically, the subject is a human.

A therapeutically effective amount of a compound as described herein used in the present invention may vary depending upon the route of administration and dosage form. Effective amounts of invention compounds typically fall in the range of about 0.001 up to 100 mg/kg/day, and more typically in the range of about 0.05 up to 10 mg/kg/day. Typically, the compound or compounds used in the instant invention are selected to provide a formulation that exhibits a high therapeutic index. The therapeutic index is the dose ratio between toxic and therapeutic effects which can be expressed as the ratio between LD₅₀ and ED₅₀. The LD₅₀ is the dose lethal to 50% of the population and the ED₅₀ is the dose therapeutically effective in 50% of the population. The LD₅₀ and ED₅₀ are determined by standard pharmaceutical procedures in animal cell cultures or experimental animals.

### E. Routes of Administration

The instant invention also provides for pharmaceutical compositions and medicaments which may be prepared by combining one or more compounds described herein, pharmaceutically acceptable salts thereof, stereoisomers thereof, tautomers thereof, or solvates thereof, with pharmaceutically acceptable carriers, excipients, binders, diluents or the like to inhibit or treat primary and/or metastatic prostate cancers. Such compositions can be in the form of, for example, granules, powders, tablets, capsules, syrup, suppositories, injections, emulsions, elixirs, suspensions or solutions. The instant compositions can be formulated for various routes of administration, for example, by oral, parenteral, topical, rectal, nasal, or via implanted reservoir. Parenteral or systemic administration includes, but is not limited to, subcutaneous, intravenous, intraperitoneal, and intramuscular injections. The following dosage forms are given by way of example and should not be construed as limiting the instant invention.

For oral, buccal, and sublingual administration, powders, suspensions, granules, tablets, pills, capsules, gelcaps, and caplets are acceptable as solid dosage forms. These can be prepared, for example, by mixing one or more compounds of the instant invention, or pharmaceutically acceptable salts or tautomers thereof, with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or antioxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as, but not limited to, an oil, water, an alcohol, and combinations of these. Pharmaceutically suitable surfactants, suspending agents, emulsifying agents, may be added for oral or parenteral administration.

As noted above, suspensions may include oils. Such oils include, but are not limited to, peanut oil, sesame oil, cottonseed oil, com oil and olive oil. Suspension preparations may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Typically, the oil or fatty acid is non-volatile, including natural or synthetic oils, fatty acids, mono-, di- or tri-glycerides.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

For rectal administration, the pharmaceutical formulations and medicaments may be in the form of a suppository, an ointment, an enema, a tablet or a cream for release of compound in the intestines, sigmoid flexure and/or rectum. Rectal suppositories are prepared by mixing one or more compounds of the instant invention, or pharmaceutically acceptable salts or tautomers of the compound, with acceptable vehicles, for example, cocoa butter or polyethylene glycol, which is present in a solid phase at normal storing temperatures, and present in a liquid phase at those temperatures suitable to release a drug inside the body, such as in the rectum. Oils may also be employed in the preparation of formulations of the soft gelatin type and suppositories. Water, saline, aqueous dextrose and related sugar solutions, and glycerols may be employed in the preparation of suspension formulations which may also contain suspending agents such as pectins, carbomers, methyl cellulose, hydroxypropyl cellulose or carboxymethyl cellulose, as well as buffers and preservatives.

Compounds of the invention may be administered to the lungs by inhalation through the nose or mouth. Suitable pharmaceutical formulations for inhalation include solutions, sprays, dry powders, or aerosols containing any appropriate solvents and optionally other compounds such as, but not limited to, stabilizers, antimicrobial agents, antioxidants, pH modifiers, surfactants, bioavailability modifiers and combinations of these. Formulations for inhalation administration contain as excipients, for example, lactose, polyoxyethylene-9-lauryl ether, glycocholate and deoxycholate. Aqueous and nonaqueous aerosols are typically used for delivery of inventive compounds by inhalation.

Ordinarily, an aqueous aerosol is made by formulating an aqueous solution or suspension of the compound together with conventional pharmaceutically acceptable carriers and stabilizers. The carriers and stabilizers vary with the requirements of the particular compound, but typically include nonionic surfactants (TWEENs, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions. A nonaqueous suspension (e.g., in a fluorocarbon propellant) can also be used to deliver compounds of the invention.

Aerosols containing compounds for use according to the present invention are conveniently delivered using an inhaler, atomizer, pressurized pack or a nebulizer and a suitable propellant, e.g., without limitation, pressurized dichlorodifluoromethane, trichlorofluoromethane, dichlorotetrafluoroethane, nitrogen, air, or carbon dioxide. In the case of a pressurized aerosol, the dosage unit may be controlled by providing a valve to deliver a metered amount. Capsules and cartridges of, for example, gelatin for use in an inhaler or insufflator may be formulated containing a powder mix of the compound and a suitable powder base such as lactose or starch.

Delivery of aerosols of the present invention using sonic nebulizers is advantageous because nebulizers minimize exposure of the agent to shear, which can result in degradation of the compound.

For nasal administration, the pharmaceutical formulations and medicaments may be a spray, nasal drops or aerosol containing an appropriate solvent(s) and optionally other compounds such as, but not limited to, stabilizers, antimicrobial agents, antioxidants, pH modifiers, surfactants, bioavailability modifiers and combinations of these. For administration in the form of nasal drops, the compounds maybe formulated in oily solutions or as a gel. For administration of nasal aerosol, any suitable propellant may be used including compressed air, nitrogen, carbon dioxide, or a hydrocarbon based low boiling solvent.

Dosage forms for the topical (including buccal and sublingual) or transdermal administration of compounds of the invention include powders, sprays, ointments, pastes, creams, lotions, gels, solutions, and patches. The active component may be mixed under sterile conditions with a pharmaceutically-acceptable carrier or excipient, and with any preservatives, or buffers, which may be required. Powders and sprays can be prepared, for example, with excipients such as lactose, talc, silicic acid, aluminum hydroxide, calcium silicates and polyamide powder, or mixtures of these substances. The ointments, pastes, creams and gels may also contain excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Transdermal patches have the added advantage of providing controlled delivery of a compound of the invention to the body. Such dosage forms can be made by dissolving or dispersing the agent in the proper medium. Absorption enhancers can also be used to increase the flux of the inventive compound across the skin. The rate of such flux can be controlled by either providing a rate controlling membrane or dispersing the compound in a polymer matrix or gel.

Besides those representative dosage forms described above, pharmaceutically acceptable excipients and carriers are generally known to those skilled in the art and are thus included in the instant invention. Such excipients and carriers are described, for example, in “Remingtons Pharmaceutical Sciences” Mack Pub. Co., New Jersey (1991), which is incorporated herein by reference.

The formulations of the invention may be designed to be short-acting, fast-releasing, long-acting, and sustained-releasing as described below. Thus, the pharmaceutical formulations may also be formulated for controlled release or for slow release.

The instant compositions may also comprise, for example, micelles or liposomes, or some other encapsulated form, or may be administered in an extended release form to provide a prolonged storage and/or delivery effect. Therefore, the pharmaceutical formulations and medicaments may be compressed into pellets or cylinders and implanted intramuscularly or subcutaneously as depot injections or as implants such as stents. Such implants may employ known inert materials such as silicones and biodegradable polymers.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant invention.

### F. Methods of Combined Therapy

Tumor cell resistance to chemotherapeutic agents represents a major problem in clinical oncology. Compositions and methods of the present invention provide means of ameliorating this problem by effectively administering a combined therapy approach. However, it should be noted that traditional combination therapy may be employed in combination with the compositions of the present invention. For example, in some embodiments of the present invention, Rho-inhibiting compositions of the present invention may be used before, after, or in combination with the traditional therapies.

To kill cells, inhibit cell growth, or metastasis, or angiogenesis, or otherwise reverse or reduce the malignant phenotype of tumor cells using the methods and compositions of the present invention in combination therapy, one contacts a “target” cell with the compositions described herein and at least one other agent. These compositions are provided in a combined amount effective to kill or inhibit proliferation of the cell. This process may involve contacting the cells with the immunotherapeutic agent and the agent(s) or factor(s) at the same time. This may be achieved by contacting the cell with a single composition or pharmacological formulation that includes both agents, or by contacting the cell with two distinct compositions or formulations, at the same time.

Alternatively, rho-inhibiting treatment may precede or follow the other agent treatment by intervals ranging from minutes to weeks. In embodiments where the other agent and immunotherapy are applied separately to the cell, one would generally ensure that a significant period of time did not expire between the time of each delivery, such that the agent and rho-inhibiting composition would still be able to exert an advantageously combined effect on the cell. In such instances, it is contemplated that cells are contacted with both modalities within about 12-24 hours of each other and, more preferably, within about 6-12 hours of each other, with a delay time of only about 12 hours being most preferred. In some situations, it may be desirable to extend the time period for treatment significantly, however, where several days (2 to 7) to several weeks (1 to 8) lapse between the respective administrations.

In some embodiments, more than one administration of the immunotherapeutic composition of the present invention or the other agent is utilized. Various combinations may be employed, where the rho-inhibiting composition is “A” and the other agent is “B”, as exemplified below:

A/B/A, B/A/B, B/B/A, A/A/B, B/A/A, A/B/B, B/B/B/A, B/B/A/B,
A/A/B/B, A/B/A/B, A/B/B/A, B/B/A/A, B/A/B/A, B/A/A/B, B/B/B/A,
A/A/A/B, B/A/A/A, A/B/A/A, A/A/B/A, A/B/B/B, B/A/B/B, B/B/A/B.

Other combinations are contemplated. Again, to achieve cell killing, both agents are delivered to a cell in a combined amount effective to kill or disable the cell.

In some embodiments of the invention, one or more compounds of the invention and an additional active agent are administered to a subject, more typically a human, in a sequence and within a time interval such that the compound can act together with the other agent to provide an enhanced benefit relative to the benefits obtained if they were administered otherwise. For example, the additional active agents can be co-administered by co-formulation, administered at the same time or administered sequentially in any order at different points in time; however, if not administered at the same time, they should be administered sufficiently close in time so as to provide the desired therapeutic or prophylactic effect. In some embodiments, the compound and the additional active agents exert their effects at times which overlap. Each additional active agent can be administered separately, in any appropriate form and by any suitable route. In other embodiments, the compound is administered before, concurrently or after administration of the additional active agents.

In various examples, the compound and the additional active agents are administered less than about 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart or no more than 48 hours apart. In other examples, the compound and the additional active agents are administered concurrently. In yet other examples, the compound and the additional active agents are administered concurrently by co-formulation.

In other examples, the compound and the additional active agents are administered at about 2 to 4 days apart, at about 4 to 6 days apart, at about 1 week part, at about 1 to 2 weeks apart, or more than 2 weeks apart.

In certain examples, the inventive compound and optionally the additional active agents are cyclically administered to a subject. Cycling therapy involves the administration of a first agent for a period of time, followed by the administration of a second agent and/or third agent for a period of time and repeating this sequential administration. Cycling therapy can provide a variety of benefits, e.g., reduce the development of resistance to one or more of the therapies, avoid or reduce the side effects of one or more of the therapies, and/or improve the efficacy of the treatment.

In other examples, one or more compound of some embodiments of the present invention and optionally the additional active agent are administered in a cycle of less than about 3 weeks, about once every two weeks, about once every 10 days or about once every week. One cycle can comprise the administration of an inventive compound and optionally the second active agent by infusion over about 90 minutes every cycle, about 1 hour every cycle, about 45 minutes every cycle, about 30 minutes every cycle or about 15 minutes every cycle. Each cycle can comprise at least 1 week of rest, at least 2 weeks of rest, at least 3 weeks of rest. The number of cycles administered is from about 1 to about 12 cycles, more typically from about 2 to about 10 cycles, and more typically from about 2 to about 8 cycles.

Courses of treatment can be administered concurrently to a subject, i.e., individual doses of the additional active agents are administered separately yet within a time interval such that the inventive compound can work together with the additional active agents. For example, one component can be administered once per week in combination with the other components that can be administered once every two weeks or once every three weeks. In other words, the dosing regimens are carried out concurrently even if the therapeutics are not administered simultaneously or during the same day.

The additional active agents can act additively or, more typically, synergistically with the inventive compound(s). In one example, one or more inventive compound is administered concurrently with one or more second active agents in the same pharmaceutical composition. In another example, one or more inventive compound is administered concurrently with one or more second active agents in separate pharmaceutical compositions. In still another example, one or more inventive compound is administered prior to or subsequent to administration of a second active agent. The invention contemplates administration of an inventive compound and a second active agent by the same or different routes of administration, e.g., oral and parenteral. In certain embodiments, when the inventive compound is administered concurrently with a second active agent that potentially produces adverse side effects including, but not limited to, toxicity, the second active agent can advantageously be administered at a dose that falls below the threshold that the adverse side effect is elicited.

Other factors that may be used in combination therapy with the rho-inhibiting compositions of the present invention include, but are not limited to, factors that cause DNA damage such as γ-rays, X-rays, and/or the directed delivery of radioisotopes to tumor cells. Other forms of DNA damaging factors are also contemplated such as microwaves and UV-irradiation. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells. The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

In some embodiments of the present invention, the regional delivery of rho-inhibiting compositions of some embodiments the present invention to patients with cancers is utilized to maximize the therapeutic effectiveness of the delivered agent. Similarly, the chemo- or radiotherapy may be directed to a particular, affected region of the subject's body. Alternatively, systemic delivery of the immunotherapeutic composition and/or the agent may be appropriate in certain circumstances, for example, where extensive metastasis has occurred.

In addition to combining the rho-inhibiting compositions of some embodiments of the present invention with chemo- and radiotherapies, it also is contemplated that traditional gene therapies are used. For example, targeting of p53 or p16 mutations along with treatment of the rho-inhibiting compositions of the present invention provides an improved anti-cancer treatment. The present invention contemplates the co-treatment with other tumor-related genes including, but not limited to, p21, Rb, APC, DCC, NF-I, NF-2, BCRA2, p16, FHIT, WT-I, MEN-I, MEN-II, BRCA1, VHL, FCC, MCC, ras, myc, neu, raf erb, src, fms, jun, trk, ret, gsp, hst, bcl, and abl.

An attractive feature of the present invention is that the therapeutic compositions may be delivered to local sites in a patient by a medical device. Medical devices that are suitable for use in the present invention include known devices for the localized delivery of therapeutic agents. Such devices include, but are not limited to, catheters such as injection catheters, balloon catheters, double balloon catheters, microporous balloon catheters, channel balloon catheters, infusion catheters, perfusion catheters, etc., which are, for example, coated with the therapeutic agents or through which the agents are administered; needle injection devices such as hypodermic needles and needle injection catheters; needleless injection devices such as jet injectors; coated stents, bifurcated stents, vascular grafts, stent grafts, etc.; and coated vaso-occlusive devices such as wire coils.

Exemplary devices are described in U.S. Pat. Nos. 5,935,114; 5,908,413; 5,792,105; 5,693,014; 5,674,192; 5,876,445; 5,913,894; 5,868,719; 5,851,228; 5,843,089; 5,800,519; 5,800,508; 5,800,391; 5,354,308; 5,755,722; 5,733,303; 5,866,561; 5,857,998; 5,843,003; and 5,933,145; the entire contents of which are incorporated herein by reference. Exemplary stents that are commercially available and may be used in the present application include the RADIUS (SCIMED LIFE SYSTEMS, Inc.), the SYMPHONY (Boston Scientific Corporation), the Wallstent (Schneider Inc.), the PRECEDENT II (Boston Scientific Corporation) and the NIR (Medinol Inc.). Such devices are delivered to and/or implanted at target locations within the body by known techniques.

In some embodiments, composition embodiments of the present invention are co-administered with an anti-cancer agent (e.g., chemotherapeutic). In some embodiments, method embodiments of the present invention encompass co-administration of an anti-cancer agent (e.g., chemotherapeutic). The present invention is not limited by type of anti-cancer agent co-administered. Indeed, a variety of anti-cancer agents are contemplated to be useful in the present invention including, but not limited to, Acivicin; Aclarubicin; Acodazole Hydrochloride; Acronine; Adozelesin; Adriamycin; Aldesleukin; Alitretinoin; Allopurinol Sodium; Altretamine; Ambomycin; Ametantrone Acetate; Aminoglutethimide; Amsacrine; Anastrozole; Annonaceous Acetogenins; Anthramycin; Asimicin; Asparaginase; Asperlin; Azacitidine; Azetepa; Azotomycin; Batimastat; Benzodepa; Bexarotene; Bicalutamide; Bisantrene Hydrochloride; Bisnafide Dimesylate; Bizelesin; Bleomycin Sulfate; Brequinar Sodium; Bropirimine; Bullatacin; Busulfan; Cabergoline; Cactinomycin; Calusterone; Caracemide; Carbetimer; Carboplatin; Carmustine; Carubicin Hydrochloride; Carzelesin; Cedefingol; Celecoxib; Chlorambucil; Cirolemycin; Cisplatin; Cladribine; Crisnatol Mesylate; Cyclophosphamide; Cytarabine; Dacarbazine; DACA (N-[2-(Dimethyl-amino)ethyl]acridine-4-carboxamide); Dactinomycin; Daunorubicin Hydrochloride; Daunomycin; Decitabine; Denileukin Diftitox; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil 1131; Etoposide; Etoposide Phosphate; Etoprine; Fadrozole Hydrochloride; Fazarabine; Fenretinide; Floxuridine; Fludarabine Phosphate; Fluorouracil; 5-FdUMP; Fluorocitabine; Fosquidone; Fostriecin Sodium; FK-317; FK-973; FR-66979; FR-900482; Gemcitabine; Geimcitabine Hydrochloride; Gemtuzumab Ozogamicin; Gold Au 198; Goserelin Acetate; Guanacone; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-1a; Interferon Gamma-1b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Methoxsalen; Metoprine; Meturedepa; Mitindomide; Mitocarcin; Mitocromin; Mitogillin; Mitomalcin; Mitomycin; Mytomycin C; Mitosper; Mitotane; Mitoxantrone Hydrochloride; Mycophenolic Acid; Nocodazole; Nogalamycin; Oprelvekin; Ormaplatin; Oxisuran; Paclitaxel; Pamidronate Disodium; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rituximab; Rogletimide; Rolliniastatin; Safingol; Safingol Hydrochloride; Samarium/Lexidronam; Semustine; Simtrazene; Sparfosate Sodium; Sparsomycin; Spirogermanium Hydrochloride; Spiromustine; Spiroplatin; Squamocin; Squamotacin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Thymitaq; Tiazofurin; Tirapazamine; Tomudex; TOP-53; Topotecan Hydrochloride; Toremifene Citrate; Trastuzumab; Trestolone Acetate; Triciribine Phosphate; Trimetrexate; Trimetrexate Glucuronate; Triptorelin; Tubulozole Hydrochloride; Uracil Mustard; Uredepa; Valrubicin; Vapreotide; Verteporfin; Vinblastine; Vinblastine Sulfate; Vincristine; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride; 2-Chlorodeoxyadenosine; 2′-Deoxyformycin; 9-aminocamptothecin; raltitrexed; N-propargyl-5,8-dideazafolic acid; 2-chloro-2′-arabino-fluoro-2′-deoxyadenosine; 2-chloro-2′-deoxyadenosine; anisomycin; trichostatin A; hPRL-G129R; CEP-751; linomide; sulfur mustard; nitrogen mustard (mechlorethamine); cyclophosphamide; melphalan; chlorambucil; ifosfamide; busulfan; N-methyl-N-nitrosourea (MNU); N,N′-Bis(2-chloroethyl)-N-nitrosourea (BCNU); N-(2-chloroethyl)-N′-cyclohex-yl-N-nitrosourea (CCNU); N-(2-chloroethyl)-N′-(trans-4-methylcyclohexyl-N-nitrosourea (MeCCNU); N-(2-chloroethyl)-N′-(diethyl)ethylphosphonate-N-nit-rosourea (fotemustine); streptozotocin; diacarbazine (DTIC); mitozolomide; temozolomide; thiotepa; mitomycin C; AZQ; adozelesin; Cisplatin; Carboplatin; Ormaplatin; Oxaliplatin; C1-973; DWA 2114R; JM216; JM335; Bis (platinum); tomudex; azacitidine; cytarabine; gemcitabine; 6-Mercaptopurine; 6-Thioguanine; Hypoxanthine; teniposide; 9-amino camptothecin; Topotecan; CPT-11; Doxorubicin; Daunomycin; Epirubicin; darubicin; mitoxantrone; losoxantrone; Dactinomycin (Actinomycin D); amsacrine; pyrazoloacridine; all-trans retinol; 14-hydroxy-retro-retinol; all-trans retinoic acid; N-(4-Hydroxyphenyl) retinamide; 13-cis retinoic acid; 3-Methyl TTNEB; 9-cis retinoic acid; fludarabine (2-F-ara-AMP); and 2-chlorodeoxyadenosine (2-Cda).

Other anti-cancer agents include: Antiproliferative agents (e.g., Piritrexim Isothionate), Antiprostatic hypertrophy agent (e.g., Sitogluside), Benign prostatic hypertrophy therapy agents (e.g., Tamsulosin Hydrochloride), Prostate growth inhibitor agents (e.g., Pentomone), and Radioactive agents: Fibrinogen I 125; Fludeoxyglucose F 18; Fluorodopa F 18; Insulin I 125; Insulin I 131; Iobenguane I 123; Iodipamide Sodium I 131; Iodoantipyrine I 131; Iodocholesterol I 131; Iodohippurate Sodium I 123; Iodohippurate Sodium I 125; Iodohippurate Sodium I 131; Iodopyracet I 125; Iodopyracet I 131; Iofetamine Hydrochloride I 123; Iomethin I 125; Iomethin I 131; Iothalamate Sodium I 125; Iothalamate Sodium I 131; Iotyrosine I 131; Liothyronine I 125; Liothyronine I 131; Merisoprol Acetate Hg 197; Merisoprol Acetate Hg 203; Merisoprol Hg 197; Selenomethionine Se 75; Technetium Tc 99m Antimony Trisulfide Colloid; Technetium Tc 99m Bicisate; Technetium Tc 99m Disofenin; Technetium Tc 99m Etidronate; Technetium Tc 99m Exametazime; Technetium Tc 99m Furifosmin; Technetium Tc 99m Gluceptate; Technetium Tc 99m Lidofenin; Technetium Tc 99m Mebrofenin; Technetium Tc 99m Medronate; Technetium Tc 99m Medronate Disodium; Technetium Tc 99m Mertiatide; Technetium Tc 99m Oxidronate; Technetium Tc 99m Pentetate; Technetium Tc 99m Pentetate Calcium Trisodium; Technetium Tc 99m Sestamibi; Technetium Tc 99m Siboroxime; Technetium Tc 99m Succimer; Technetium Tc 99m Sulfur Colloid; Technetium Tc 99m Teboroxime; Technetium Tc 99m Tetrofosmin; Technetium Tc 99m Tiatide; Thyroxine I 125; Thyroxine I 131; Tolpovidone I 131; Triolein I 125; Triolein I 131.

Another category of anti-cancer agents is anti-cancer Supplementary Potentiating Agents, including: Tricyclic anti-depressant drugs (e.g., imipramine, desipramine, amitryptyline, clomipramine, trimipramine, doxepin, nortriptyline, protriptyline, amoxapine and maprotiline); non-tricyclic anti-depressant drugs (e.g., sertraline, trazodone and citalopram); C⁺⁺ antagonists (e.g., verapamil, nifedipine, nitrendipine and caroverine); Calmodulin inhibitors (e.g., prenylamine, trifluoroperazine and clomipramine); Amphotericin B; Triparanol analogues (e.g., tamoxifen); antiarrhythmic drugs (e.g., quinidine); antihypertensive drugs (e.g., reserpine); Thiol depleters (e.g., buthionine and sulfoximine) and Multiple Drug Resistance reducing agents such as Cremaphor EL.

Still other anticancer agents are those selected from the group consisting of: annonaceous acetogenins; asimicin; rolliniastatin; guanacone, squamocin, bullatacin; squamotacin; taxanes; paclitaxel; gemcitabine; methotrexate FR-900482; FK-973; FR-66979; FK-317; 5-FU; FUDR; FdUMP; Hydroxyurea; Docetaxel; discodermolide; epothilones; vincristine; vinblastine; vinorelbine; meta-pac; irinotecan; SN-38; 10-OH campto; topotecan; etoposide; adriamycin; flavopiridol; Cis-Pt; carbo-Pt; bleomycin; mitomycin C; mithramycin; capecitabine; cytarabine; 2-Cl-2′ deoxyadenosine; Fludarabine-PO₄; mitoxantrone; mitozolomide; Pentostatin; and Tomudex.

One particularly preferred class of anticancer agents are taxanes (e.g., paclitaxel and docetaxel). Another important category of anticancer agent is annonaceous acetogenin.

Other cancer therapies include hormonal manipulation. In some embodiments, the anti-cancer agent is tamoxifen or the aromatase inhibitor arimidex (i.e., anastrozole).

### G. In Vitro Toxicology

In some embodiments of the present invention, to gain a general perspective into the safety of a particular rho-inhibiting composition of an embodiment of the present invention, toxicity testing is performed. Toxicological information may be derived from numerous sources including, but not limited to, historical databases, in vitro testing, and in vivo animal studies.

In vitro toxicological methods have gained popularity in recent years due to increasing desires for alternatives to animal experimentation and an increased perception to the potential ethical, commercial, and scientific value. In vitro toxicity testing systems have numerous advantages including improved efficiency, reduced cost, and reduced variability between experiments. These systems also reduce animal usage, eliminate confounding systemic effects (e.g., immunity), and control environmental conditions.

Although any in vitro testing system may be used with the present invention, the most common approach utilized for in vitro examination is the use of cultured cell models. These systems include freshly isolated cells, primary cells, or transformed cell cultures. Cell culture as the primary means of studying in vitro toxicology is advantageous due to rapid screening of multiple cultures, usefulness in identifying and assessing toxic effects at the cellular, subcellular, or molecular level. In vitro cell culture methods commonly indicate basic cellular toxicity through measurement of membrane integrity, metabolic activities, and subcellular perturbations. Commonly used indicators for membrane integrity include cell viability (cell count), clonal expansion tests, trypan blue exclusion, intracellular enzyme release (e.g. lactate dehydrogenase), membrane permeability of small ions (K⁺, Ca²⁺), and intracellular Ala accumulation of small molecules (e.g., ⁵¹Cr, succinate). Subcellular perturbations include monitoring mitochondrial enzyme activity levels via, for example, the MTT test, the WST1 assay, determining cellular adenine triphosphate (ATP) levels, neutral red uptake into lysosomes, and quantification of total protein synthesis. Metabolic activity indicators include glutathione content, lipid peroxidation, and lactate/pyruvate ratio. It should be noted that compounds having toxicity may still be employed in appropriate circumstances, e.g., for research use.

1. MTT Assay and WST-1 Assay

The MTT assay is a fast, accurate, and reliable methodology for obtaining cell viability measurements. The MTT assay was first developed by Mosmann (See, e.g., Mosmann, J. Immunol. Meth., 65:55 (1983)). It is a simple colorimetric assay numerous laboratories have utilized for obtaining toxicity results (See e.g., Kuhlmann et al., Arch. Toxicol., 72:536 (1998)). Briefly, the mitochondria produce ATP to provide sufficient energy for the cell. In order to do this, the mitochondria metabolize pyruvate to produce acetyl CoA. Within the mitochondria, acetyl CoA reacts with various enzymes in the tricarboxylic acid cycle resulting in subsequent production of ATP. One of the enzymes particularly useful in the MTT assay is succinate dehydrogenase. MTT (3-(4,5-dimethylthiazol-2-yl)-2 diphenyl tetrazolium bromide) is a yellow substrate that is cleaved by succinate dehydrogenase forming a purple formazan product. The alteration in pigment identifies changes in mitochondria function. Nonviable cells are unable to produce formazan, and therefore, the amount produced directly correlates to the quantity of viable cells. Absorbance at 540 nm is utilized to measure the amount of formazan product.

An alternative to the MTT assay is the WST-1 assay, which similarly is based on measurement of metabolic activity to measure toxin effects on mammalian cells but uses a different substrate, 4-[3-(4-iodophenyl)-2-(4-nitrophenyl)-2H-5-tetrazolio]-1,3-benzene disulfonate (Dietrich et al., Appl. Environ. Microbiol., 65:4470 (1999); Kau et al., Curr. Microbiol., 44:106 (2002); Scobie et al., PNAS, 100:5170 (2003); Moravek et al., FEMS Microbiol. Lett., 257:293 (2006); Ngamwongsatit et al., J. Microbiol. Methods, 73:211 (2008); each herein incorporated by reference in its entirety). In the WST-1 assay, mitochondrial succinate-tetrazolium reductase reacts with the WST-1 reagent to produce water-soluble formazan dye. This water solubility is an advantage over the classical MTT assay, as the product of the WST-1 assay can be quantified in 0.4-4 h without additional solubilization steps (Ngamwongsatit et al., J. Microbiol. Methods, 73:211 (2008); herein incorporated by reference in its entirety). Therefore, in some cases, WST-1 assays may be use preferentially to MTT assays if handling time is a concern (e.g., in high-throughput screens).

The results of the in vitro tests can be compared to in vivo toxicity tests in order to extrapolate to live animal conditions. Typically, acute toxicity from a single dose of the substance is assessed. Animals are monitored over 14 days for any signs of toxicity (increased temperature, breathing difficulty, death, etc). Traditionally, the standard of acute toxicity is the median lethal dose (LD₅₀), which is the predicted dose at which half of the treated population would be killed. The determination of this dose occurs by exposing test animals to a geometric series of doses under controlled conditions. Other tests include subacute toxicity testing, which measures the animal's response to repeated doses of the composition for no longer than 14 days. Subchronic toxicity testing involves testing of a repeated dose for 90 days. Chronic toxicity testing is similar to subchronic testing but may last for over a 90-day period. In vivo testing can also be conducted to determine toxicity with respect to certain tissues. For example, in some embodiments of the present invention, tumor toxicity (e.g., effect of the compositions of the present invention on the survival of tumor tissue) is determined (e.g., by detecting changes in the size and/or growth of tumor cells or tissues).

### EXAMPLES

The following examples are provided in order to demonstrate and further illustrate certain preferred embodiments and aspects of the present invention and are not to be construed as limiting the scope thereof.

### Example 1

### Synthetic Schemes, Preparations, and Compound Synthesis Reactions

The following schemes are representative of the synthetic procedures used to prepare examples of the invention.

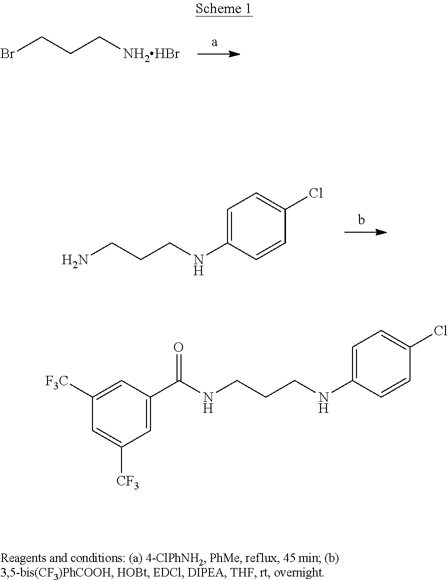

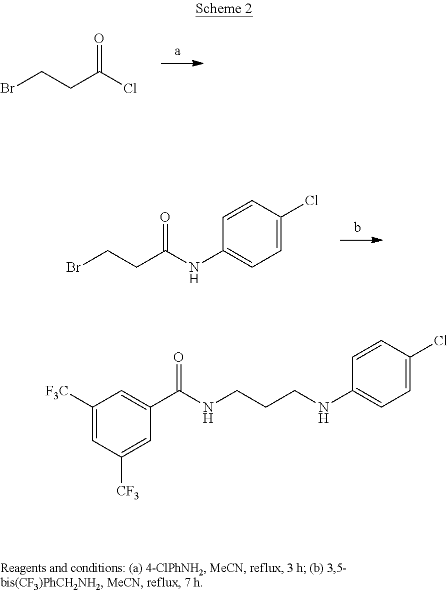

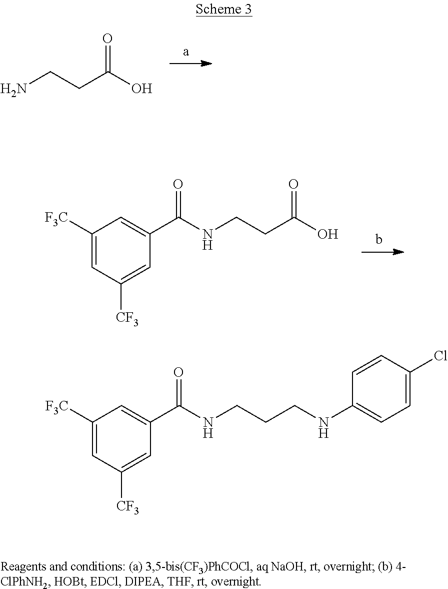

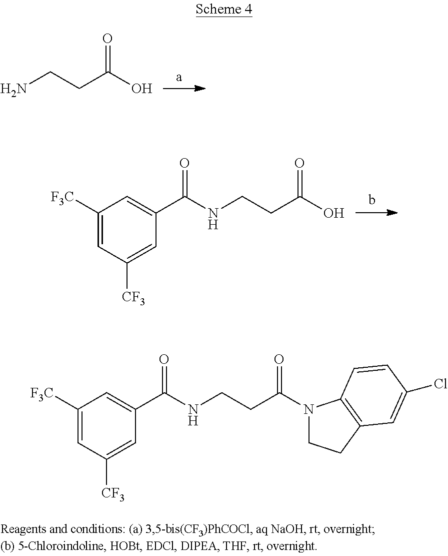

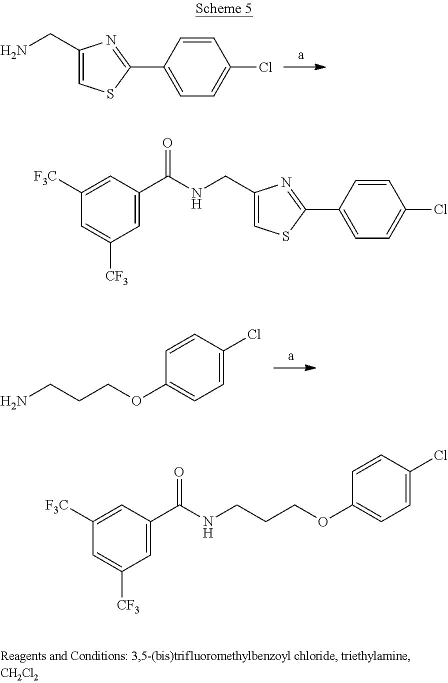

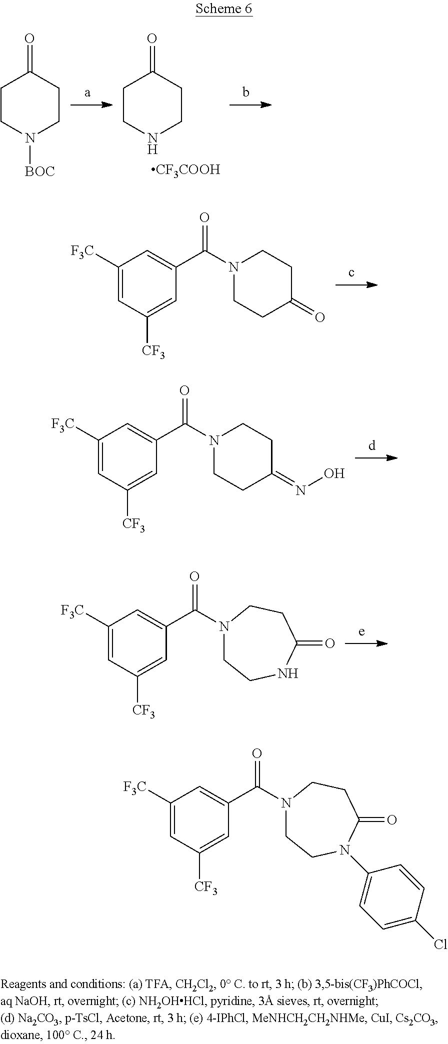

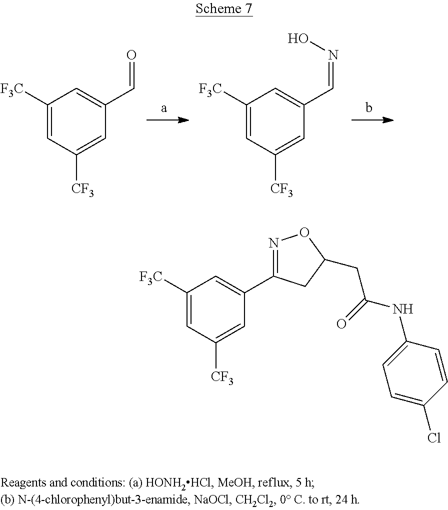

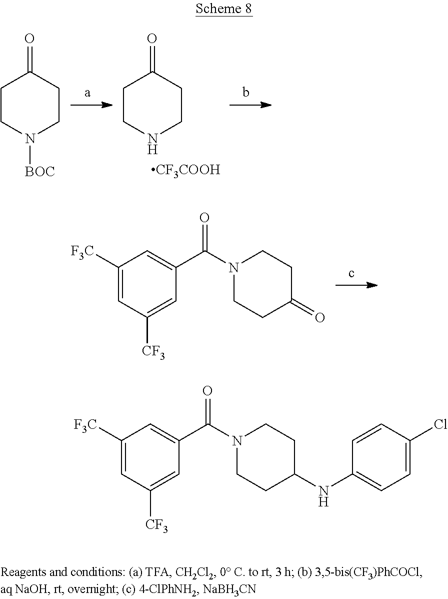

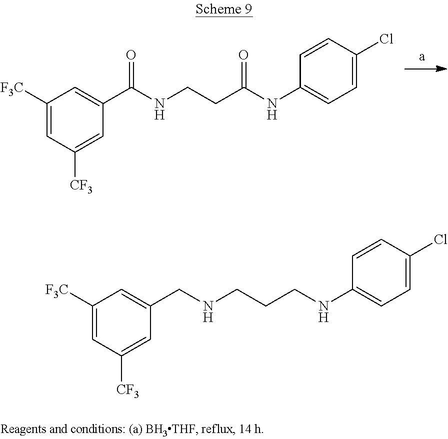

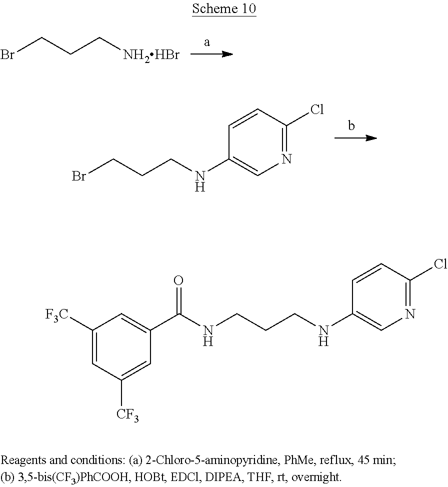

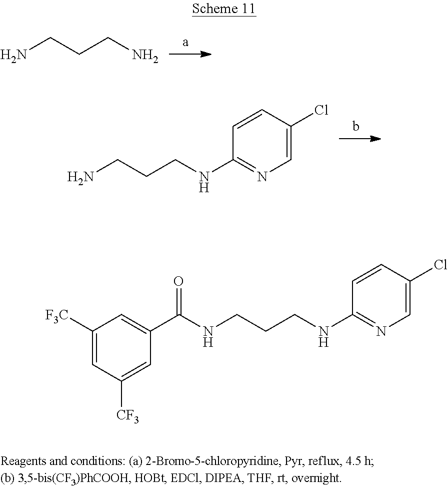

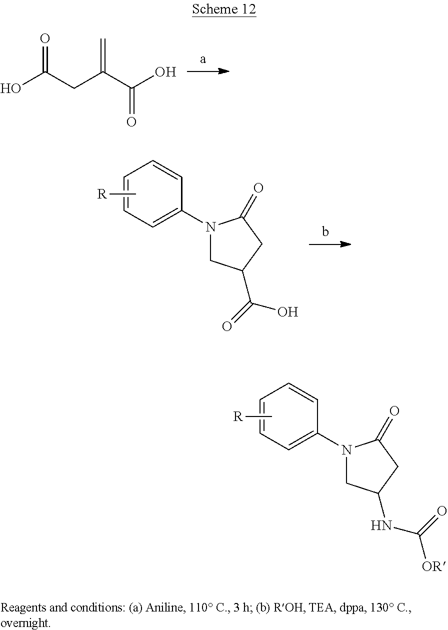

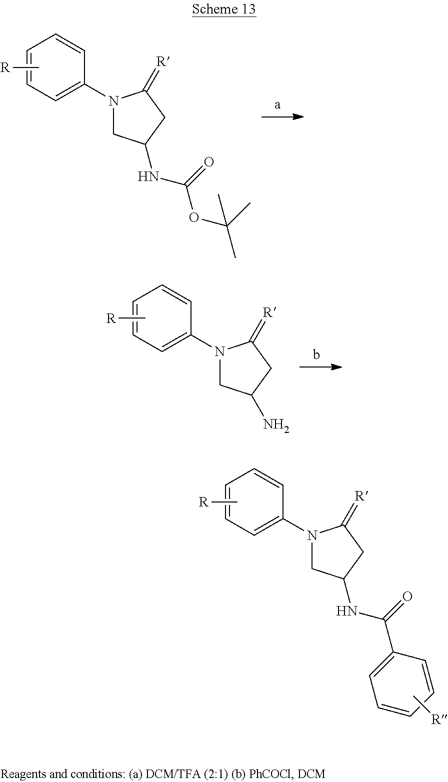

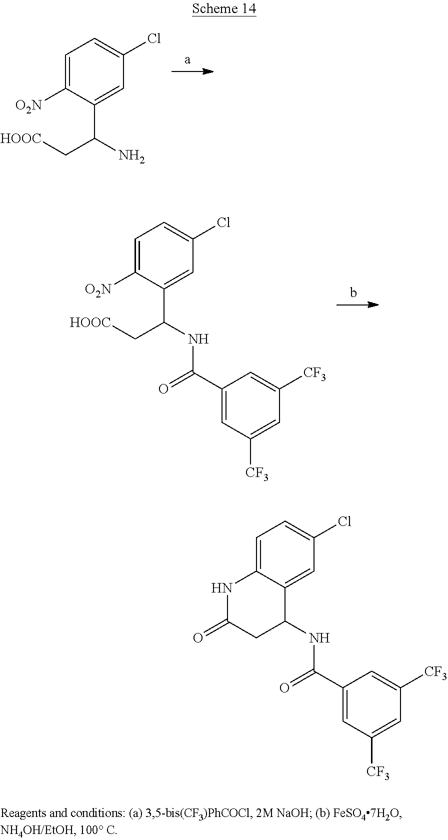

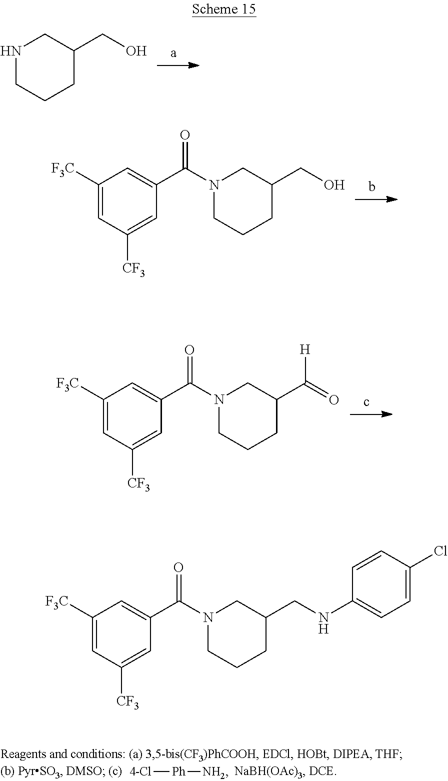

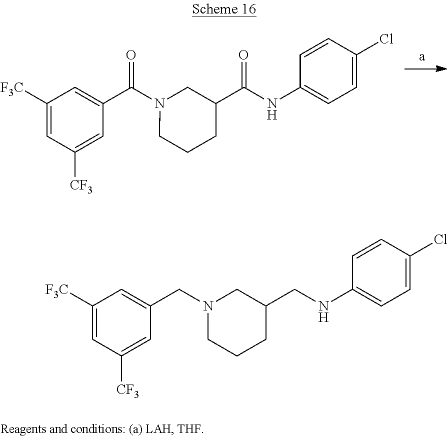

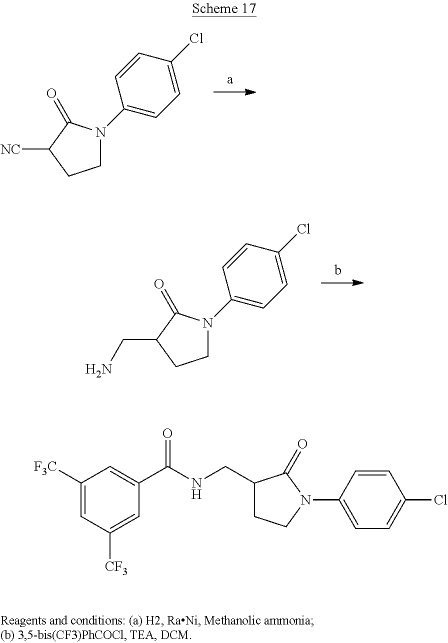

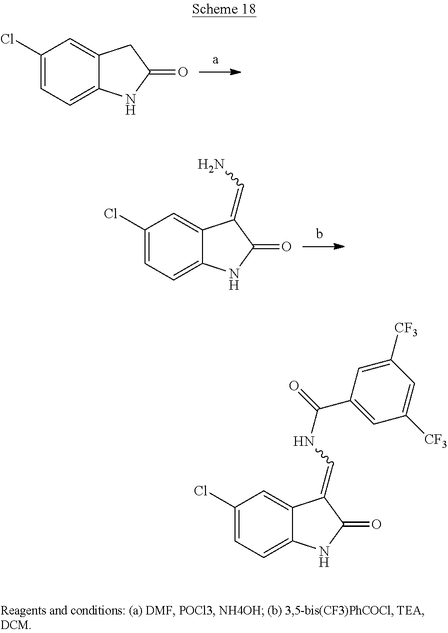

### Preparation 1: 2-(3,5-bis(trifluoromethyl)benzamidooxy)acetic acid

Carboxymethoxylamine hemihydrochloride (0.186 g, 1.7 mmol) was dissolved in 3.4 ml of 2N aq NaOH solution and treated with 3,5-bis(trifluoromethyl)benzoyl chloride (0.433 ml, 2.4 mmol). After stirring overnight, the white opaque solution was extracted with dichloromethane and the aqueous layer was acidified to pH 2 with 2N aq HCl. The acidic solution was extracted with EtOAc two times and the combined EtOAc layers were washed with brine, dried over MgSO₄, filtered, and concentrated to a white solid. The white solid crude product was used in the next step.

### Preparation 2: 4-(3,5-bis(trifluoromethyl)benzamido)butanoic acid

4-aminobutyric acid (0.175 g, 1.70 mmol) was dissolved in 2N aq NaOH (3.40 ml) solution and treated with 3,5-bis(trifluoromethyl)benzoyl chloride (0.464 ml, 2.55 mmol). After stirring overnight, the solution was extracted with dichloromethane and the aqueous layer was acidified to pH 2 with 2N aq HCl. The acidic solution was extracted with EtOAc two times and the combined EtOAc layers were washed with brine, dried over MgSO₄, filtered, and concentrated to a white solid.

### Preparation 3: 2-(3,5-bis(trifluoromethyl)benzamido)acetic acid

The title compound was prepared using the same procedure as described in Preparation 2 from glycine and 3,5-bis(trifluoromethyl)benzoyl chloride.

### Preparation 4: 1-(3,5-bis(trifluoromethyl)benzoyl)piperidine-3-carboxylic acid

Nipecotic acid (153 mg) was suspended in dichloromethane (11.8 mL) and to the suspension was added 3,5-bis(trifluoromethyl)benzoyl chloride (0.215 mL) followed by Triethylamine (0.165 mL). The mixture was stirred vigorously overnight. The mixture was concentrated and partitioned between 1N HCl (10 mL) and EtOAc (2×10 mL). The organic layer was washed with brine (20 mL), dried (MgSO₄), and concentrated to yield 438 mg of the title compound as a yellow oil.

### Preparation 5: 1-(3,5-bis(trifluoromethyl)benzoyl)piperidine-4-carboxylic acid

The title compound was prepared using the procedure described for Preparation 2 from isonipecotic acid and 3,5-bis(trifluoromethyl)benzoyl chloride.

### Preparation 6: 2-(4-chlorobenzamidooxy)acetic acid

The title compound was prepared using the procedure described for Preparation 2 from carboxymethoxylamine hemihydrochloride and 4-chlorobenzoylchloride.

### Preparation 7: 1-(3,5-bis(trifluoromethyl)benzoyl)pyrrolidine-2-carboxylic acid

The title compound was prepared using the procedure described for Preparation 2 from proline and 3,5-bis(trifluoromethyl)benzoyl chloride.

### Preparation 8: 1-(4-chlorobenzoyl)piperidine-4-carboxylic acid

Isonipecotic acid (0.602 g, 4.66 mmol) was dissolved in 2N NaOH (aq) (4.66 ml) and treated with 4-chlorobenzoyl chloride (0.300 ml, 2.331 mmol) dissolved in dichloromethane (4.66 ml). The biphasic solution was stirred overnight. The dichloromethane layer was then discarded and the aqueous layer was acidified to pH 2 with 2N aq HCl. The acidic solution was extracted with EtOAc two times and the combined organics were washed with brine, dried over Na₂SO₄, filtered, and concentrated to a white solid.

### Preparation 9: 3-(3,5-bis(trifluoromethyl)benzamido)propanoic acid

3-aminopropanoic acid (214 mg) was dissolved in 2M NaOH (4.0 mL) and to the solution was added 3,5-bis(trifluoromethyl)benzoyl chloride (0.362 mL). The reaction mixture was stirred overnight at room temperature. After stirring overnight, the solution was washed with dichloromethane (20 mL). The aqueous layer was acidified to pH 2 using 2N HCl (aq). The acidic solution was extracted with EtOAc (2×30 mL) and the organic layer was washed with brine (30 mL), dried (MgSO₄), and concentrated to afford 590 mg of the title compound as a white solid.

### Preparation 10: (3,5-bis(trifluoromethyl)phenyl)(4-(hydroxyimino)piperidin-1-yl)methanone

1-(3,5-bis(trifluoromethyl)benzoyl)piperidin-4-one (PCT patent application PCT/EP2001/006305, Jun. 6, 2001; herein incorporated by reference in its entirety) (67 mg) was dissolved in pyridine (0.5 mL) and to the solution was added molecular sieves (3A, 8-12 mesh, 144 mg). The mixture was stirred at room temperature for 10 minutes, and hydroxylamine hydrochloride (35 mg) was added. The reaction was allowed to stir overnight at room temperature. The reaction mixture was then poured through celite. The filtrate was diluted with water (2 mL), and extracted with ethyl acetate (15 mL). The organic layer was dried (MgSO₄) and concentrated to afford 57 mg of the title compound as a white solid.

### Preparation 11: 1-(3,5-bis(trifluoromethyl)benzoyl)-1,4-diazepan-5-one

(3,5-bis(trifluoromethyl)phenyl)(4-(hydroxyimino)piperidin-1-yl)methanone (190 mg) was dissolved in acetone (2.3 mL) and to the solution was added a solution of sodium carbonate (171 mg) in water (0.531 mL), and the mixture was stirred for 10 minutes. A solution of p-toluenesulfonyl chloride (153 mg) in acetone (0.574 mL) was added slowly while stirring. The mixture was stirred for 3 h and the acetone was removed and water (10 mL) was added. The aqueous mixture was extracted with dichloromethane (3×20 mL), and the organic layer was dried (MgSO₄) and concentrated. The crude product was purified by column chromatography (CH₂Cl₂/methanolic ammonia, 19/1) to afford 116 mg of the title compound as a pale yellow solid.

### Preparation 12: N¹-(3-chloro-4-methoxyphenyl)-propane-1,3-diamine

3-chloro-p-anisidine (662 mg) was dissolved in toluene (4.25 ml) and to the solution was added 3-bromoproylamine hydrobromide (306 mg). The reaction refluxed for 45 minutes and was cooled to room temperature. The reaction was filtered and the solid was washed with toluene. The solid was dried and treated with 5% aqueous NaOH (5 mL) and was extracted with dichloromethane (2×20 mL). The organic layer was washed with water (20 mL), dried (MgSO₄), and concentrated. The crude product was purified using column chromatography (CH₂Cl₂/methanolic ammonia, 9/1 to 3/1) to afford 97 mg of the title compound as an oil.

### Preparation 13: N¹-(4-chloro-3-methoxyphenyl)propane-1,3-diamine

5-amino-2-chloroanisole (448 mg) was dissolved in toluene (3.0 mL) and to the solution was added 3-bromopropylamine hydrobromide (414 mg). The reaction was allowed to reflux for 1 hour and the mixture was cooled to room temperature. The solution was filtered and the solid was washed with toluene, and dried, and treated with 15% aqueous NaOH (5 mL). The aqueous solution was then extracted with dichloromethane (2×20 mL) and the organic layer was washed with water (20 mL), dried (MgSO₄), and concentrated. The crude product was purified using column chromatography (CH₂Cl₂/methanolic ammonia, 9/1 to 4/1 to 10/3) to afford 61 mg of the title compound as an oil.

### Preparation 14: 3-(3,5-bis(trifluoromethyl)_{b} enzamido)benzoic acid

m-Aminobenzoic acid (162 mg) was suspended in dichloromethane (11.8 mL) and to the suspension was added 3,5-bis(trifluoromethyl)benzoyl chloride (0.215 mL) followed by Triethylamine (0.165 mL). The mixture was stirred vigorously overnight. The mixture was concentrated and partitioned in between 1N HCl (10 mL) and EtOAc (2×10 mL). The organic layer was washed with brine (20 mL), dried (Na₂SO₄), and concentrated to yield 429 mg of the title compound as a white solid.

### Preparation 15: 2-(3,5-bis(trifluoromethyl)benzamido)benzoic acid

Anthranilic acid (162 mg) was suspended in dichloromethane (11.8 mL) and to the suspension was added 3,5-bis(trifluoromethyl)benzoyl chloride (0.215 mL) followed by Triethylamine (0.164 mL). The mixture was allowed to stir overnight at room temperature. Mixture was concentrated and partitioned between 2N HCl (10 mL) and EtOAc (3×10 mL). The organic layer was washed with brine (15 mL), dried (MgSO₄), and concentrated to afford 424 mg of the title compound as a white solid.

### Preparation 16: tert-butyl 3-(4-chlorophenylcarbamoyl)pyrrolidine-1-carboxylate

1-(tert-butoxycarbonyl)pyrrolidine-3-carboxylic acid (108 mg) was dissolved in anhydrous THF (1.7 mL) and to the solution was added 4-chloroaniline (70 mg), followed by 1-Hydroxybenzotriazole hydrate (81 mg), N-(3-Dimethylaminopropyl)-N′-ethyl-carbodiimide hydrochloride (115 mg), and N,N-Diisopropylethylamine (0.105 mL). The solution was allowed to stir at room temperature overnight under nitrogen. Ethyl acetate (5 mL) was added and the organic layer was washed with NaHCO₃ (8 mL), followed by 1N HCl (8 mL) and brine (8 mL), dried (MgSO₄), and concentrated to afford 129 mg of the title compound as an amber oil.

### Preparation 17: N-(4-chlorophenyl)pyrrolidine-3-carboxamide

tert-butyl 3-(4-chlorophenylcarbamoyl)pyrrolidine-1-carboxylate (129 mg) was dissolved in dichloromethane (3.5 mL) and the solution was cooled to 0° C. Trifluoroacetic acid (1.04 mL) was added dropwise and the solution was warmed and stirred at room temperature for 2 h. Solvent was removed to afford the title compound in quantitative yield.

### Preparation 18: 2-(1-(3,5-bis(trifluoromethyl)benzoyl)piperidin-2-yl)acetic acid

2-(piperidin-2-yl)acetic acid hydrochloride (259 mg) was dissolved in 2M NaOH (2.4 mL) and dichloromethane (2.4 mL). The reaction mixture was stirred vigorously, and to the mixture was added 3,5-bis(trifluoromethyl)benzoyl chloride (0.216 mL). The mixture was stirred vigorously overnight. The CH₂Cl₂ layer was removed and the solution was acidified to pH 2 and was extracted using EtOAc (2×20 mL). The organic layer was washed with brine (20 mL), dried (MgSO₄) and concentrated. The crude material was purified using column chromatography (4/6/0.5 EtOAc/Hexanes/AcOH) to afford 91 mg of the title compound as a yellow oil.

### Preparation 19. N¹-(6-chloropyridin-3-yl)propane-1,3-diamine

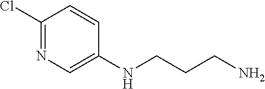 [0162] Oura, Takeshi. Preparation of cyclic nitroguanidines as insecticides. European Patent JP09316056 A, December 1997; herein incorporated by reference in its entirety.

### Preparation 20. N¹-(5-chloropyridin-2-yl)propane-1,3-diamine

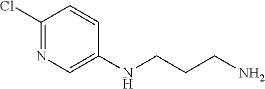 [0164] Eur. J. Med. Chem. 1989, 24, 249-257; herein incorporated by reference in its entirety.

### Preparation 21. -(4-chlorophenyl)-5-oxopyrrolidine-3-carboxylic acid

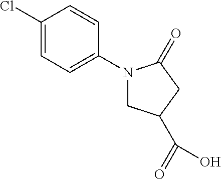 [0166] Tetrahedron 2007, 63, 3049-3056; herein incorporated by reference in its entirety.

### Preparation 22. tert-butyl (1-(4-chlorophenyl)-5-oxopyrrolidin-3-yl)carbamate

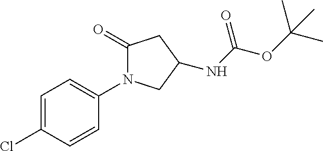

Title compound was prepared using the procedure described in Compound 45 using tert-Butanol (0.238 mL) to afford 183 mg of title compound as a white solid.

### Preparation 23. 4-amino-1-(4-chlorophenyl)pyrrolidin-2-one

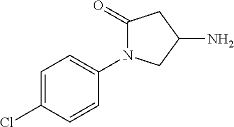 [0169] J. Heterocyclic Chem. 1976, 13, 529-532; herein incorporated by reference in its entirety.

### Preparation 24. 3-amino-3-(5-chloro-2-nitrophenyl)propanoic acid

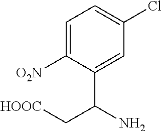 [0171] J. Med. Chem. 1992, 35, 2155-2162; herein incorporated by reference in its entirety.

### Preparation 25. 3-(3,5-bis(trifluoromethyl)benzamido)-3-(5-chloro-2-nitrophenyl)propanoic acid

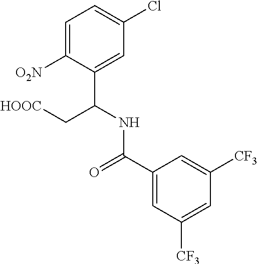

3-amino-3-(5-chloro-2-nitrophenyl)propanoic acid (41 mg) was dissolved in 2M NaOH (0.335 mL). To the stirring solution was added 3,5-bis(trifluoromethyl)benzoyl chloride (0.033 mL). The mixture was stirred overnight at room temperature under nitrogen. The mixture was acidified to pH ˜2 using 2M aq. HCl. The acidic mixture was then filtered and the resulting solid was rinsed with water. The solid was then dried to afford 50 mg of title compound as a fluffy white solid. Compound was used without further purification.

### Preparation 26. 1-(4-chloro-3-methoxyphenyl)-5-oxopyrrolidine-3-carboxylic acid

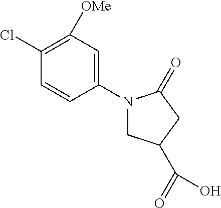

Title compound was prepared using the procedure described in Preparation 21 using Itaconic acid (100 mg) and 5-Amino-2-chloroanisole (121 mg) to afford 130 mg of title compound as light purple solid.

### Preparation 27. tert-butyl (1-(4-chloro-3-methoxyphenyl)-5-oxopyrrolidin-3-yl)carbamate

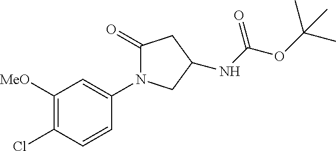

Title compound was prepared using the procedure described in Preparation 22 using 1-(4-chloro-3-methoxyphenyl)-5-oxopyrrolidine-3-carboxylic acid (131 mg) afford 78 mg of title compound as an off-white solid.

### Preparation 28. 4-amino-1-(4-chloro-3-methoxyphenyl)pyrrolidin-2-one

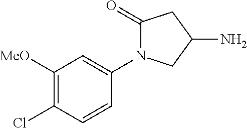

tert-butyl (1-(4-chloro-3-methoxyphenyl)-5-oxopyrrolidin-3-yl)carbamate (20 mg) was dissolved in Dichloromethane (0.391 mL) and cooled to −20° C., and to the stirring solution was added Trifluoroacetic acid (0.196 mL) for 2 hours. The mixture was then warmed to −10° C., and was stirred for 2 additional hours. 2M NaOH (5 mL) was cooled to −10° C., and the reaction mixture was slowly added to the NaOH. The aqueous layer was then extracted using dichloromethane (3×5 mL), and the organic layer was then dried (MgSO₄), filtered, and concentrated to afford 12 mg of title compound as a yellow oil. Product was carried on without further purification.

### Preparation 29. 1-(4-chlorophenyl)-2-oxopyrrolidine-3-carbonitrile

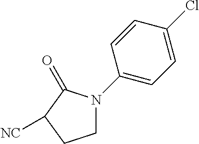 [0177] J. Heterocyclic Chem. 2007, 44, 201-203; herein incorporated by reference in its entirety.

### Preparation 30. 3-(aminomethyl)-1-(4-chlorophenyl)pyrrolidin-2-one

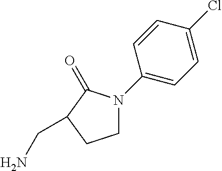

1-(4-chlorophenyl)-2-oxopyrrolidine-3-carbonitrile (10 mg) was dissolved in 1M Methanolic ammonia (1.0 mL), and to the solution was added a spatula tip of Raney Nickel catalyst. The mixture was placed on a Parr shaker at 25 psi at room temperature overnight. After shaking overnight, the mixture was filtered through a plug of celite topped with MgSO₄. The celite was rinsed repeatedly with methanol and concentrated. The crude material was triturated with chloroform to afford 6 mg of title compound as a brown solid. No further purification was performed.

### Preparation 31. 3-(aminomethylene)-5-chloroindolin-2-one

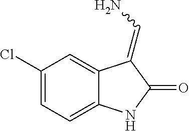

N,N-Dimethylformamide (0.878 mL) and Phosphorus oxychloride (0.061 mL) were heated to 45° C. 5-Chlorooxindole (100 mg) was added to the mixture, and the temperature was held at 45° C. for 1 hr. The reaction was then cooled to 0° C. and to the cooled mixture was added Ammonium hydroxide (17.55 mL). The mixture was warmed to room temperature and allowed to stir for 30 minutes. Reaction mixture was then extracted with ethyl acetate (3×20 mL), dried (MgSO₄), and concentrated. The crude material was triturated with dichloromethane to afford 58 mg of title compound as a yellow solid.

### Preparation 32. 1-(3-chloro-4-methoxyphenyl)-5-oxopyrrolidine-3-carboxylic acid

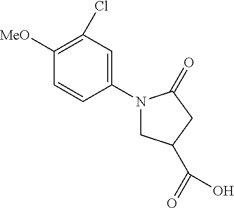

Title compound was prepared using the procedure described for Preparation 21 using Itaconic acid (100 mg) and 3-Chloro-p-anisidine (121 mg) to afford 106 mg of title compound as a tan solid.

### Preparation 33. tert-butyl (1-(3-chloro-4-methoxyphenyl)-5-oxopyrrolidin-3-yl)carbamate

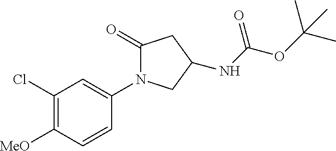

Title compound was prepared using the procedure described in Compound 45 using 1-(3-chloro-4-methoxyphenyl)-5-oxopyrrolidine-3-carboxylic acid (52 mg) and tent-Butanol (0.238 mL) to afford 21 mg of title compound as a crystalline white solid.

### Preparation 34. 4-amino-1-(3-chloro-4-methoxyphenyl)pyrrolidin-2-one

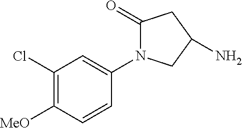

tert-butyl (1-(3-chloro-4-methoxyphenyl)-5-oxopyrrolidin-3-yl)carbamate (21 mg) was dissolved in Dichloromethane (0.411 mL) and was cooled to 0° C. Trifluoroacetic acid (0.205 mL) was added, and stirring continued at 0° C. for 2 hours. The solution was then slowly added to cooled 1M aq NaOH (5 mL). The mixture was then extracted with dichloromethane (3×5 mL) and was concentrated to afford 108 mg of title compound as an off-white solid. No further purification was performed.

### Preparation 35. (3,5-bis(trifluoromethyl)phenyl)(3-(hydroxymethyl)piperidin-1-yl)methanone

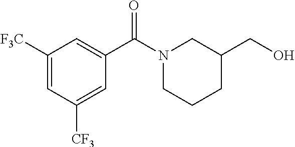

3,5-bis(trifluoromethyl)benzoic acid (271 mg) was dissolved in Tetrahydrofuran (3.4 mL), and to the solution was added Piperidin-3-yl methanol (115 mg) followed by N-(3-Dimethylaminopropyl)-N′-ethyl-carbodiimide hydrochloride (230 mg), 1-Hydroxybenzotriazole hydrate (162 mg), and N,N-Diisopropylethylamine (0.210 mL). The reaction mixture was allowed to stir at room temperature under nitrogen overnight. The reaction mixture was diluted with ethyl acetate (15 mL) and was washed with sat. aq. NaHCO₃ (20 mL). The organics were then washed with brine (20 mL), dried (MgSO₄), filtered, and concentrated. The crude material was purified using column chromatography (Ethyl acetate/Hexanes, 1/1) and concentrated to afford 282 mg of title compound as a clear oil.

### Preparation 36. 1-(3,5-bis(trifluoromethyl)benzoyl)piperidine-3-carbaldehyde

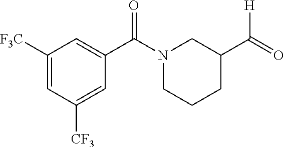

To a stirring solution of (3,5-bis(trifluoromethyl)phenyl)(3-(hydroxymethyl)piperidin-1-yl)methanone (51 mg) and N,N-Diisopropylethylamine (0.163 mL) in Dichloromethane (0.167 mL) at 0° C. was added Sulfur trioxide pyridine complex (75 mg) in anhydrous DMSO (0.571 mL). The reaction was stirred at 0° C. for 20 minutes and then warmed to room temperature. Reaction continued stirring at room temperature for 2.5 h and then water (3 mL) was added. The aqueous layer was extracted with dichloromethane (3×3 mL) and the organic layers were combined and washed with 1N aq. HCl (3 mL) followed by brine (3 mL), dried (MgSO₄), filtered, and concentrated. The crude material was purified using column chromatography (Hexanes/EtOAc, 3/2) and concentrated to afford 40 mg of title compound as an off-white solid.

### Preparation 37. tert-butyl (1-(4-chlorophenyl)pyrrolidin-3-yl)carbamate

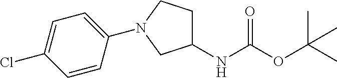

A solution of racemic BINAP (4.6 mg) and tris(dibenzylideneacetone)dipalladium(0) (7.4 mg) in Toluene (4.0 mL) was stirred under nitrogen. The mixture was heated to 90° C. and allowed to stir for 10 minutes. The mixture was then cooled to 40° C. and 3-(tert-Butoxycarbonylamino)-pyrrolidine (75 mg), 1-chloro-4-iodobenzene (106 mg), and Sodium tert-butoxide (62 mg) were added. The mixture was then heated at 80° C. overnight. The reaction mixture was cooled to room temperature, diluted with ethyl acetate, and filtered through a plug of celite which was then rinsed repeatedly with ethyl acetate. The filtrate was then concentrated. The crude material was purified using column chromatography (Hexanes/EtOAc, 9/1) and concentrated to afford 21 mg of title compound as a tan solid.

¹H NMR (500 MHz, CDCl₃) δ 7.17, 6.46, 4.71, 4.36, 3.55-3.51, 3.42-3.37, 3.31-3.27, 3.13, 2.32-2.25, 1.95-1.94, 1.45.

### Preparation 38. 1-(4-chlorophenyl)pyrrolidin-3-amine

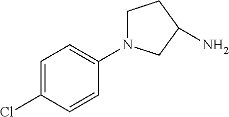

tert-butyl (1-(4-chlorophenyl)pyrrolidin-3-yl)carbamate (21 mg) was dissolved in Dichloromethane (0.472 mL) and placed in an ice bath at approximately 0° C. Trifluoroacetic acid (0.236 mL) was slowly added, and the solution continued to stir at 0° C. After 2 hours, the reaction mixture was added to 2M aq NaOH (5 mL, cooled to bath temperature of 0° C.). The cold mixture was then extracted with dichloromethane (4×5 mL). The combined organic layers were dried (MgSO₄), filtered, and concentrated to afford 12 mg of title compound as a yellow solid. No further purification was performed.

### Compound Synthesis Reactions

### Reaction 1 (Compound 1): N-(2-(4-chlorophenylamino)-2-oxoethoxy)-3,5-bis(trifluoromethyl)benzamide

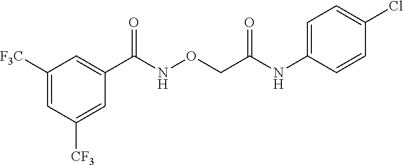

To a solution of crude 2-(3,5-bis(trifluoromethyl)benzamidooxy)acetic acid (0.563 g, 1.70 mmol) and 4-chloroaniline (0.217 g, 1.70 mmol) in dry Tetrahydrofuran (5.67 ml) was added 1-Hydroxybenzotriazole hydrate (0.230 g, 1.700 mmol), N-(3-Dimethylaminopropyl)-n′-ethyl-carbodiimide hcl (0.326 g, 1.700 mmol), and N,N-Diisopropylethylamine (0.297 ml, 1.700 mmol). The mixture was stirred at room temperature overnight. The resulting light yellow solution was then diluted with EtOAc and washed with sat. aq. NaHCO₃, then 1N aq HCl. The organics were washed with brine, dried over MgSO₄, filtered, and concentrated to a light pink solid. The crude material was purified by flash column chromatography eluting with 50% EtOAc in heptanes to isolate the product as a white solid in 75% yield over 2 steps. ¹H NMR (500 MHz, DMSO-d₆) δ ppm: 12.73, 10.55, 8.42, 8.35, 7.70, 7.41, 4.65.

### Reaction 2 (Compound 2): N-(4-(4-chlorophenylamino)-4-oxobutyl)-3,5-bis(trifluoromethyl)benzamide

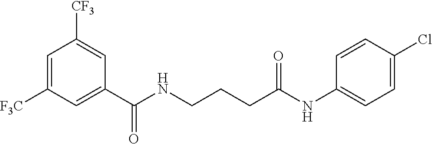

To a solution of crude 4-(3,5-bis(trifluoromethyl)benzamido)butanoic acid (0.730 g, 2.127 mmol) and 4-chloroaniline (0.217 g, 1.70 mmol) in dry tetrahydrofuran (5.67 ml) was added 1-hydroxybenzotriazole hydrate (0.276 g, 2.040 mmol), N-(3-dimethylaminopropyl)-n′-ethyl-carbodiimide hcl (0.391 g, 2.040 mmol), and N,N-diisopropylethylamine (0.356 ml, 2.040 mmol). The mixture was stirred at room temperature overnight. The light brown solution was then diluted with EtOAc and washed with sat. aq. NaHCO₃, then 1N aq HCl. The organics were washed with brine, dried over MgSO4, filtered, and concentrated to a light pink solid. The crude material was adsorbed onto silica gel and purified by flash column chromatography eluting with 50% EtOAc in heptanes to isolate the product as a white solid in 66% yield over 2 steps. ¹H NMR (500 MHz, DMSO-d₆) δ ppm: 10.0, 9.00, 8.50, 8.30, 7.59, 7.31, 3.40, 2.41, 1.90.

### Reaction 3 (Compound 3): N-(2-(4-chlorophenylamino)-2-oxoethyl)-3,5-bis(trifluoromethyl)benzamide

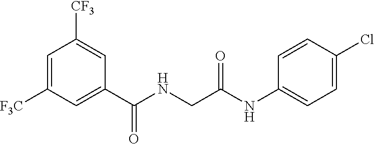

The title compound was prepared using the procedure described for Compound 2 from crude 2-(3,5-bis(trifluoromethyl)benzamido)acetic acid and 4-chloroaniline, as a light yellow solid in 60% yield over 2 steps. ¹H NMR (500 MHz, DMSO-d₆) δ ppm: 10.25, 9.46, 8.57, 8.38, 7.65, 7.39, 4.14.

### Reaction 4 (Compound 4): 1-(3,5-bis(trifluoromethyl)benzoyl)-N-(4-chlorophenyl)piperidine-3-carboxamide

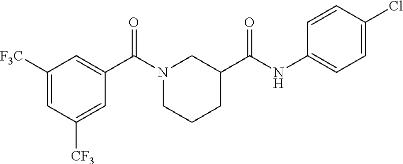

The title compound was prepared using the procedure described for Compound 2 from crude 1-(3,5-bis(trifluoromethyl)benzoyl)piperidine-3-carboxylic acid and 4-chloroaniline, as a cream foam in 38% yield over 2 steps. ¹H NMR (500 MHz, CDCl₃) δ ppm. 8.55, 8.38, 8.13, 7.99, 7.89, 7.60, 7.32, 4.24, 3.95, 3.53, 3.43, 2.74, 2.30, 2.03, 1.71, 1.58. MS (EI) m/z 479 (M+1).

### Reaction 5 (Compound 5): 1-(3,5-bis(trifluoromethyl)benzoyl)-N-(4-chlorophenyl)piperidine-4-carboxamide

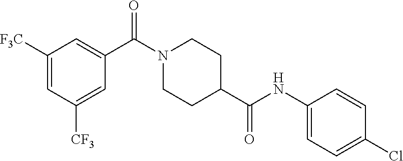

The title compound was prepared using the procedure described for Compound 2 from 1-(3,5-bis(trifluoromethyl)benzoyl)piperidine-4-carboxylic acid and 4-chloroaniline, in 31% yield over 2 steps. ¹H NMR (500 MHz, DMSO-d₆) δ ppm: 10.08, 8.22, 8.15, 7.64, 7.36, 4.53, 3.51, 3.20, 2.92, 2.63, 1.93, 1.74-1.66. MS (EI) m/z 479 (M+1).

### Reaction 6 (Compound 6): N-(2-(3,5-bis(trifluoromethyl)phenylamino)-2-oxoethoxy)-4-chlorobenzamide

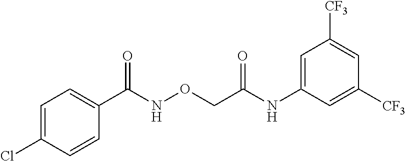

The title compound was prepared using the procedure described for Compound 2 from 2-(4-chlorobenzamidooxy)acetic acid and 3,5-bis(trifluoromethyl)aniline in 28% yield over 2 steps. ¹H NMR (500 MHz, DMSO-d₆) δ ppm: 12.36, 11.10, 7.83, 7.81, 7.57, 4.65.

### Reaction 7 (Compound 7): 1-(3,5-bis(trifluoromethyl)benzoyl)-N-(4-chlorophenyl)pyrrolidine-2-carboxamide

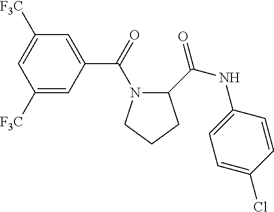

The title compound was prepared using the procedure described for Compound 2 from 1-(3,5-bis(trifluoromethyl)benzoyl)pyrrolidine-2-carboxylic acid and 4-chloroaniline in 22% yield over 2 steps. ¹H NMR (500 MHz, DMSO-d₆) δ ppm: 10.23, 9.98, 8.53, 8.44, 8.33, 8.30, 8.22, 8.11, 8.00, 7.67, 7.65, 7.40, 7.38, 7.36, 7.34, 7.30, 7.29, 4.59, 4.34, 3.67, 3.59-3.54, 2.33-2.31, 1.99-1.92, 1.91-1.82.

### Reaction 8 (Compound 8): N-(3,5-bis(trifluoromethyl)phenyl)-1-(4-chlorobenzoyl)piperidine-4-carboxamide

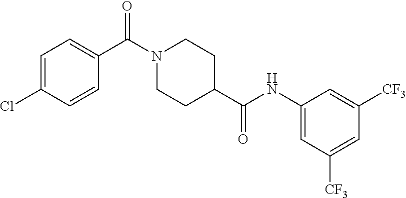

To a solution of 1-(4-chlorobenzoyl)piperidine-4-carboxylic acid (0.624 g, 2.33 mmol) and 3,5-bistrifluoromethyl aniline (0.327 ml, 2.097 mmol) in dry tetrahydrofuran (7.77 ml) was added 1-hydroxybenzotriazole hydrate (0.378 g, 2.80 mmol), N-(3-dimethylaminopropyl)-n′-ethyl-carbodiimide HCl (0.536 g, 2.80 mmol), and N,N-diisopropylethylamine (0.488 ml, 2.80 mmol). The mixture was stirred at room temperature overnight. The solution was then diluted with EtOAc and washed with sat. aq. NaHCO₃, then 1N aq HCl. The organics were washed with brine, dried over Na₂SO₄, filtered, and concentrated to a light yellow oil. The crude material was purified by flash column chromatography eluting with 25% to 70% EtOAc in hexanes to isolate the product as a white foam in 6% yield over 2 steps. H NMR (500 MHz, DMSO-d₆) δ ppm: 10.62, 8.29, 7.76, 7.53, 7.52, 7.44, 7.43, 4.48, 3.62, 3.15, 2.91, 2.67, 1.94, 1.81, 1.62.

MS (EI) m/z 479 (M+1).

### Reaction 9 (Compound 9): N-(2-(3-chloro-4-methoxyphenylamino)-2-oxoethoxy)-3,5-bis(trifluoromethyl)benzamide

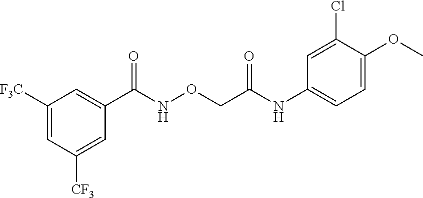

2-(3,5-bis(trifluoromethyl)benzamidooxy)acetic acid (0.189 g, 0.571 mmol) and 3-chloro-p-anisidine (0.075 g, 0.476 mmol) was dissolved in dry THF (1.586 ml). The solution was treated with EDC (0.109 g, 0.571 mmol), HOBT (0.077 g, 0.571 mmol), and DIPEA (0.099 ml, 0.571 mmol) then stirred overnight. The reaction mixture was diluted with EtOAc, washed with saturated NaHCO₃ solution, 1N HCl solution, brine and dried over Na₂SO₄. The organics were concentrated to an orange oil and purified by flash column chromatography eluting with 0% to 2% MeOH/DCM. Then recrystallized light brown oil with hexanes, EtO₂. Isolated tan solid in 11% yield over 2 steps. H NMR (500 MHz, DMSO-d₆) δ ppm: 12.74, 10.55, 8.43, 8.34, 7.85, 7.52, 7.15, 4.60, 3.83. MS (EI) m/z 471 (M+1)

### Reaction 10 (Compound 10): N-(2-(3-chlorophenylamino)-2-oxoethoxy)-3,5-bis(trifluoromethyl)benzamide

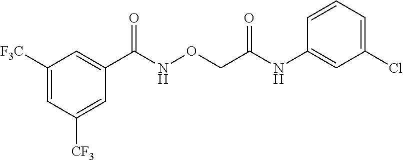

2-(3,5-bis(trifluoromethyl)benzamidooxy)acetic acid (371 mg) was dissolved in anhydrous THF (4.2 mL) and to the solution was added 3-chloroaniline (0.118 mL), followed by 1-hydroxybenzotriazole hydrate (182 mg), N-(3-dimethylaminopropyl)-N′-ethyl-carbodiimide hydrochloride (258 mg), and N,N-diisopropylethylamine (0.223 mL). The solution was allowed to stir at room temperature overnight under nitrogen. Ethyl acetate (15 mL) was added and the organic layer was washed with NaHCO₃ (20 mL), followed by 1N HCl (20 mL) and brine (20 mL), dried (Na₂SO₄), and concentrated. The crude product was purified by column chromatography (Hexanes/EtOAc, 4/1 to 7/3) to afford 27 mg of the title compound. ¹H NMR (500 MHz, DMSO-d₆) δ 12.70, 10.60, 8.43, 8.38, 7.90, 7.46, 7.40, 7.18, 4.62; MS (EI) m/z 441 (M+).

### Reaction 11 (Compound 11): N-(2-((4-chlorophenyl)(methyl)amino)-2-oxoethoxy)-3,5-bis(trifluoromethyl)benzamide

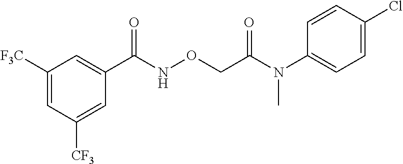

2-(3,5-bis(trifluoromethyl)benzamidooxy)acetic acid (242 mg) was dissolved in anhydrous THF (2.9 mL) and to the solution was added 4-chloro-N-methylaniline (0.088 mL), followed by 1-Hydroxybenzotriazole hydrate (118 mg), N-(3-Dimethylaminopropyl)-N′-ethyl-carbodiimide hydrochloride (168 mg), and N,N-Diisopropylethylamine (0.145 mL). The solution was allowed to stir at room temperature overnight under nitrogen. Ethyl acetate (15 mL) was added and the organic layer was washed with NaHCO₃ (20 mL), followed by 1N HCl (20 mL) and brine (20 mL), dried (Na₂SO₄), and concentrated. The crude product was purified by column chromatography (Hexanes/EtOAc, 7/3 to 2/3) to afford 34 mg of the title compound as a white solid. ¹H NMR (500 MHz, DMSO-d₆) δ 12.44, 8.39, 8.35, 7.51, 7.44, 4.38, 3.20; MS (EI) m/z 455 (M+).

### Reaction 12 (Compound 12): 1-(3,5-bis(trifluoromethyl)benzoyl)-N-(3-chloro-4-methoxyphenyl)piperidine-3-carboxamide

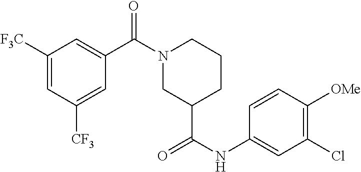

1-(3,5-bis(trifluoromethyl)benzoyl)piperidine-3-carboxylic acid (438 mg) was dissolved in anhydrous THF (4.4 mL) and to the solution was added 3-chloro-p-anisidine (186 mg), followed by 1-Hydroxybenzotriazole hydrate (192 mg), N-(3-Dimethylaminopropyl)-N′-ethyl-carbodiimide hydrochloride (272 mg), and N,N-Diisopropylethylamine (0.235 mL). The solution was allowed to stir at room temperature overnight under nitrogen. Ethyl acetate (15 mL) was added and the organic layer was washed with NaHCO₃ (20 mL), followed by 1N HCl (20 mL) and brine (20 mL), dried (MgSO₄), and concentrated. The crude product was purified by column chromatography (EtOAc/hexanes, 1/1 to 2/1 to 1/0) to afford 185 mg of the title compound as a white solid. ¹H NMR (500 MHz, DMSO-d₆) δ; 10.08, 9.86, 8.25, 8.19, 8.12, 7.82, 7.71, 7.47, 7.33, 7.13-7.06, 4.52, 4.06, 3.23-3.02, 2.03-1.49; MS (EI) m/z 509 (M+).

### Reaction 13 (Compound 13): N-(3-(3-chloro-4-methoxyphenylamino)-3-oxopropyl)-3,5-bis(trifluoromethyl)benzamide

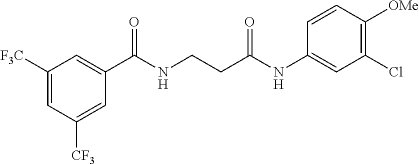

3-(3,5-bis(trifluoromethyl)benzamido)propanoic acid (323 mg) was dissolved in anhydrous THF (3.8 mL) and to the solution was added 3-chloro-p-anisidine (155 mg), followed by 1-Hydroxybenzotriazole hydrate (159 mg), N-(3-Dimethylaminopropyl)-N′-ethyl-carbodiimide hydrochloride (226 mg), and N,N-Diisopropylethylamine (0.196 mL). The solution was allowed to stir at room temperature overnight under nitrogen. Ethyl acetate (15 mL) was added and the organic layer was washed with NaHCO₃ (20 mL), followed by 1N HCl (20 mL) and brine (20 mL), dried (MgSO₄), and concentrated. The crude product was triterated with EtOAc to afford 65 mg of the title compound as a tan solid. ¹H NMR (500 MHz, DMSO-d₆) δ 10.00, 9.15, 8.49, 8.32, 7.80, 7.43, 7.41, 7.09, 3.81, 3.60, 2.63; MS (EI) m/z 491 (M+23).

### Reaction 14 (Compound 14): N-(3-(4-chlorophenylcarbamoyl)phenyl)-3,5-bis(trifluoromethyl)benzamide

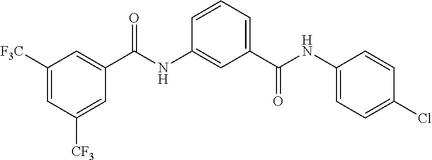

3-(3,5-bis(trifluoromethyl)benzamido)benzoic acid (429 mg) was dissolved in anhydrous THF (4.0 mL) and to the solution was added 4-chloroaniline (145 mg), followed by 1-Hydroxybenzotriazole hydrate (185 mg), N-(3-Dimethylaminopropyl)-N′-ethyl-carbodiimide hydrochloride (262 mg), and N,N-Diisopropylethylamine (0.226 mL). The solution was allowed to stir at room temperature overnight under nitrogen. Ethyl acetate (15 mL) was added and the organic layer was washed with NaHCO₃ (20 mL), followed by 1N HCl (20 mL) and brine (20 mL), dried (Na₂SO₄), and concentrated. The crude product was purified by column chromatography (Hexanes/EtOAc, 2/1 to 1/1 to 1/2) to afford 222 mg of the title compound as a brown solid. ¹H NMR (500 MHz, DMSO-d₆) δ 10.86, 10.46, 8.67, 8.41, 8.28, 8.11, 7.84, 7.77, 7.59, 7.44; MS (EI) m/z 487 (M+).

### Reaction 15 (Compound 15): N-(3-(4-chlorophenylamino)propyl)-3,5-bis(trifluoromethyl)benzamide

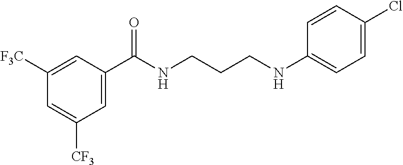

N¹-(4-chlorophenyl)propane-1,3-diamine (Syn. Comm., 1999, 29, 1819-1833) (70 mg) was dissolved in anhydrous THF (1.3 mL) and to the solution was added 3,5-bis(trifluoromethyl)benzoic acid (98 mg), followed by 1-Hydroxybenzotriazole hydrate (61 mg), N-(3-Dimethylaminopropyl)-N′-ethyl-carbodiimide hydrochloride (87 mg), and N,N-Diisopropylethylamine (0.075 mL). The solution was allowed to stir at room temperature overnight under nitrogen. Ethyl acetate (15 mL) was added and the organic layer was washed with NaHCO₃ (20 mL), followed by 1N HCl (20 mL) and brine (20 mL), dried (MgSO₄), and concentrated. The crude product was purified by column chromatography (Hexanes/EtOAc, 2/1) to afford 73 mg of the title compound as a light brown solid. ¹H NMR (500 MHz, CDCl₃) δ 8.20, 8.03, 7.16, 6.69, 6.61, 3.67, 3.29, 1.98; MS (EI) m/z 425 (M+).

### Reaction 16 (Compound 16): N-(2-(4-chlorophenylcarbamoyl)phenyl)-3,5-bis(trifluoromethyl)benzamide

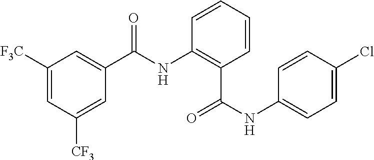

2-(3,5-bis(trifluoromethyl)benzamido)benzoic acid (426 mg) was dissolved in anhydrous THF (4.2 mL) and to the solution was added 4-chloroaniline (144 mg), followed by 1-Hydroxybenzotriazole hydrate (184 mg), N-(3-Dimethylaminopropyl)-N′-ethyl-carbodiimide hydrochloride (261 mg), and N,N-Diisopropylethylamine (0.226 mL). The solution was allowed to stir at room temperature overnight under nitrogen. Ethyl acetate (15 mL) was added and the organic layer was washed with NaHCO₃ (20 mL), followed by 1N HCl (20 mL) and brine (20 mL), dried (MgSO₄), and concentrated. The solid was triturated with dichloromethane and methanol and filtered to yield crude product. The crude product (50 mg) and 4-chloroaniline (35 mg) were combined and to the mixture was added 4-(dimethylamino)pyridine (17 mg) dissolved in 1.0 mL of pyridine. Additional pyridine (1.0 mL) was added and the solution was heated at 80° C. for 20 h. After cooling, the solution was acidified to pH 2 using 37% HCl, and the precipitate was collected and washed with water. The crude product was purified by column chromatography (Hexanes/EtOAc 2/1) and concentrated to afford 36 mg of the title compound as a white solid. ¹H NMR (500 MHz, DMSO-d₆) δ 11.27, 10.60, 8.50, 8.40, 7.97, 7.81, 7.74, 7.63, 7.40-7.37; MS (EI) m/z 509 (M+Na).

### Reaction 17 (Compound 17): (3,5-bis(trifluoromethyl)phenyl)(4-(4-chlorophenylamino)piperidin-1-yl)methanone

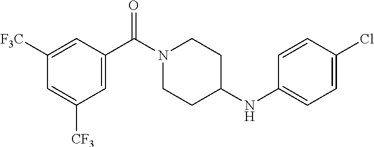

1-(3,5-bis(trifluoromethyl)benzoyl)piperidin-4-one (International patent application PCT/EP2001/006305, Jun. 6, 2001) (124 mg) was dissolved in 1,2-dichloroethane (1.3 mL) and to the solution was added 4-chloroaniline (47 mg) followed by sodium triacetoxyborohydride (110 mg) and acetic acid (0.021 mL). The mixture was stirred at room temperature overnight under nitrogen. The reaction was basified to pH 9 using 2M NaOH. The solution was extracted with diethyl ether (3×10 mL), and the ether layer was washed with brine (20 mL), dried (MgSO₄), and concentrated. The crude product was purified by column chromatography (Hexanes/EtOAc, 19/1 to 9/1 to 17/3 to 4/1 to 1/4 to 0/1) to afford 44 mg of the title compound as a clear oil. ¹H NMR (500 MHz, DMSO-d₆) δ 8.23, 8.13, 7.08, 6.62, 5.75, 4.35, 3.51-3.45, 3.23, 3.11, 2.02, 1.86, 1.42-1.35; MS (EI) m/z 451 (M+).

### Reaction 18 (Compound 18): N-((2-(4-chlorophenyl)thiazol-4-yl)methyl)-3,5-bis(trifluoromethyl)benzamide

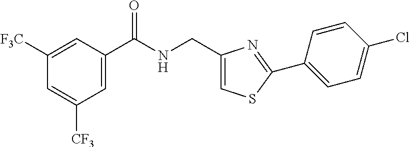

(2-(4-chlorophenyl)-1,3-thiazol-4-yl)methanamine hydrochloride (96 mg) was suspended in dichloromethane (3.7 mL) and to the suspension were added 3,5-bis(trifluoromethyl)benzoyl chloride (0.067 mL) followed by Triethylamine (0.202 mL). The solution was stirred overnight at room temperature while under nitrogen. The solution was concentrated and partitioned between 2N HCl (10 mL) and EtOAc (3×10 mL). The organic layer was washed with brine (20 mL), dried (MgSO₄), and concentrated to afford 153 mg of the title compound as a yellow solid.

¹H NMR (500 MHz, DMSO-d₆) δ 9.65, 8.59, 8.36, 7.96, 7.65, 7.58, 4.69; MS (EI) m/z 465 (M+)

### Reaction 19 (Compound 19): N-(3-(4-chlorophenoxy)propyl)-3,5-bis(trifluoromethyl)benzamide

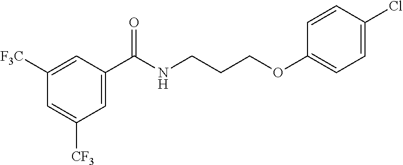

3-(4-chlorophenoxy)propan-1-amine hydrochloride (262 mg) was suspended in dichloromethane (11.8 mL) and to the suspension was added 3,5-bis(trifluoromethyl)benzoyl chloride (0.215 mL) followed by triethylamine (0.330 mL). The solution was stirred overnight at room temperature while under nitrogen. The solution was concentrated and partitioned between 2N HCl (10 mL) and EtOAc (3×10 mL). The organic layer was washed with brine (20 mL), dried (MgSO₄), and concentrated to afford 471 mg of the title compound as a yellow solid. ¹H NMR (500 MHz, DMSO-d₆) δ 9.06, 8.50, 8.33, 7.32, 6.97, 4.06, 3.48, 2.01; MS (EI) m/z 426 (M+).

### Reaction 20 (Compound 20): N-(3-(3-chloro-4-methoxyphenylamino)propyl)-3,5-bis(trifluoromethyl)benzamide

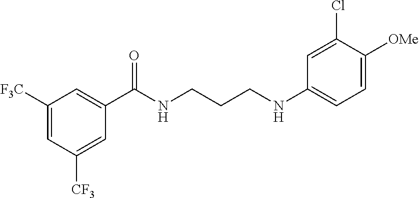

N¹-(3-chloro-4-methoxyphenyl)propane-1,3-diamine (92 mg) was dissolved in anhydrous THF (1.45 mL) and to the solution was added 3,5-bis(trifluoromethyl)benzoic acid (111 mg), followed by 1-hydroxybenzotriazole hydrate (69 mg) and N-(3-dimethylaminopropyl)-N′-ethyl-carbodiimide hydrochloride (99 mg) and N,N-diisopropylethylamine (0.090 mL). The solution was allowed to stir at room temperature overnight under nitrogen. Ethyl acetate (15 mL) was added and the organic layer was washed with NaHCO₃ (20 mL), followed by 1N HCl (20 mL) and brine (20 mL), dried (MgSO₄), and concentrated. The solid was triturated with dichloromethane affording 37 mg of the title compound as a white solid. ¹H NMR (500 MHz, DMSO-d₆) δ 9.14, 8.52, 8.34, 7.41, 7.25-7.19, 4.45, 3.83, 3.43-3.40, 3.27-3.26, 1.92-1.89; MS (EI) m/z 455 (M+).

### Reaction 21 (Compound 21): N-(3-(4-chloro-3-methoxyphenylamino)propyl)-3,5-bis(trifluoromethyl)benzamide

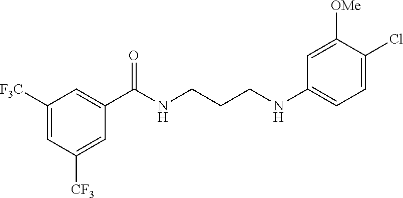

N¹-(4-chloro-3-methoxyphenyl)propane-1,3-diamine (61 mg) was dissolved in anhydrous THF (1.0 mL) and to the solution was added 3,5-bis(trifluoromethyl)benzoic acid (88 mg), followed by 1-hydroxybenzotriazole hydrate (46 mg), N-(3-dimethylaminopropyl)-N′-ethyl-carbodiimide hydrochloride (65 mg), and N,N-diisopropylethylamine (0.060 mL). The mixture was stirred overnight at room temperature while under nitrogen. Ethyl acetate (15 mL) was added, and the organic layer was washed with saturated aqueous NaHCO₃ (20 ml), followed by a wash with brine (20 mL), dried (MgSO₄), and concentrated. The crude product was purified by column chromatography (CH₂Cl₂/methanolic ammonia, 99/1 to 97/3) to afford 115 mg of the title compound as a yellow oily solid. ¹H NMR (500 MHz, CDCl₃) δ 8.21, 7.98, 7.31, 7.10, 6.21, 6.17, 6.16, 4.12, 3.81, 3.61, 3.24, 1.93; MS (EI) m/z 455 (M+).

### Reaction 22 (Compound 22): 3-(3,5-bis(trifluoromethyl)benzylamino)-N-(4-chlorophenyl)propanamide

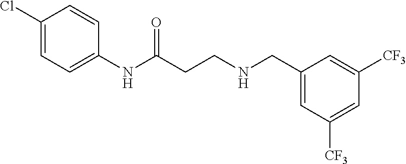

3-bromo-N-(4-chlorophenyl)propanamide (Bioorg. Med. Chem. 2006, 14, 8249-8258) (50 mg) was dissolved in acetonitrile (0.65 mL) and to the solution was added 3,5-bis(trifluoromethyl)benzylamine (171 mg). The solution was refluxed for 7 h, cooled, and diluted with water (5 mL). The pH was adjusted to 10 using 2M NaOH and was extracted with dichloromethane (3×10 mL). The organic layer was washed with NaHCO₃ (10 mL) followed by brine (10 mL), dried (MgSO₄), and concentrated. The crude product was purified by column chromatography (CH₂Cl₂/methanolic ammonia, 99/1) and concentrated to afford 43 mg of the title compound as a yellow oil. ¹H NMR (500 MHz, CDCl₃) δ 9.60, 7.82, 7.44, 7.26, 3.99, 3.04, 2.57; MS (D) m/z 425 (M+).

### Reaction 23 (Compound 23): 2-(3-(3,5-bis(trifluoromethyl)phenyl)-4,5-dihydroisoxazol-5-yl)-N-(4-chlorophenyl)acetamide

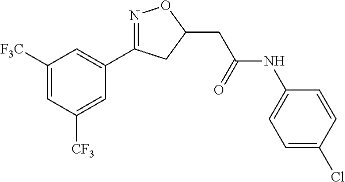

N-(4-chlorophenyl)but-3-enamide (J. Agric. Food Chem. 1992, 40, 1692-1694) (39 mg) was dissolved in dichloromethane (2.1 mL) and to the solution was added 3,5-bis(trifluoromethyl)benzaldehyde oxime (J. Agric. Food Chem. 2008, 56, 6562-6566) (75 mg). The reaction was cooled to 0° C. Sodium hypochlorite (1.134 g) was added drop wise over 20 minutes and the resulting mixture was stirred and allowed to warm to ambient temperature. The solution continued to stir overnight. Water (5 mL) was added to the solution, and the product was extracted using dichloromethane (3×5 mL). The organic layer was dried (MgSO₄) and concentrated to yield 94 mg of the title compound as an off-white solid. Physical characteristics are as follows: ¹H NMR (500 MHz, DMSO-d₆) δ 8.28, 8.24, 7.63, 7.37, 5.23-5.18, 3.75-3.69, 3.45-3.40, 2.84-2.79, 2.75-2.71; MS (EI) m/z 473 (M+Na).

### Reaction 24 (Compound 24): 1-(3,5-bis(trifluoromethyl)benzoyl)-4-(4-chlorophenyl)-1,4-diazepan-5-one

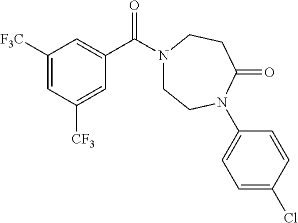

1-(3,5-bis(trifluoromethyl)benzoyl)-1,4-diazepan-5-one (27 mg), 1-chloro-4-iodobenzene (239 mg), copper(I) iodide (3.0 mg), cesium carbonate (48 mg), and N,N-dimethylethylenediamine (1.7 μL) were dissolved in dioxane (1.0 mL). The flask was evacuated and backfilled with nitrogen two times and heated to 100° C. under nitrogen. Upon reaching 100° C., the nitrogen was removed and the septum was replaced with a Teflon lined cap. The reaction was allowed to stir at 100° C. for 24 hours. The mixture was cooled to room temperature and was filtered through a 2×0.5 cm pad of silica gel eluting with 10 mL of ethyl acetate. The crude product was purified by column chromatography (EtOAc/hexanes, 1/1 to 2/1) to afford 17 mg of the title compound as a clear oil. Physical characteristics are as follows: ¹H NMR (500 MHz, CDCl₃) δ 8.01, 7.92, 7.40-7.39, 7.17, 4.16-3.70, 2.90; MS (EI) m/z 465 (M+).

### Reaction 25 (Compound 25): N-(3-(4-chlorobenzylamino)-3-oxopropyl)-3,5-bis(trifluoromethyl)benzamide

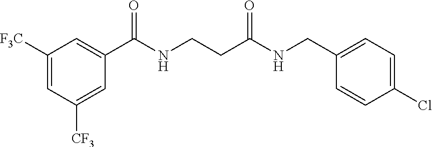

3-(3,5-bis(trifluoromethyl)benzamido)propanoic acid (104 mg) was dissolved in THF (0.750 mL) and to the solution was added 4-chlorobenzylamine (0.042 mL) followed by 1-hydroxybenzotriazole hydrate (51 mg), N-(3-dimethylaminopropyl)-N′-ethyl-carbodiimide hydrochloride (73 mg), and N,N-diisopropylethylamine (0.066 mL). The solution was stirred overnight at room temperature under nitrogen. Ethyl acetate (3 mL) was added, and the mixture was washed with saturated NaHCO₃ (5 mL) followed by a wash with 1 N HCl (5 mL) and then brine (5 mL). The organic layer was dried (MgSO₄) and concentrated. The crude product was purified by column chromatography (EtOAc/hexanes, 4/1 to 9/1) to afford 84 mg of the title compound as a white solid. Physical characteristics are as follows: ¹H NMR (500 MHz, DMSO-d₆) δ 9.12, 8.49-8.47, 8.33, 7.23, 4.25, 3.54, 2.50; MS (EI) m/z 453 (M+).

### Reaction 26 (Compound 26): 2-(1-(3,5-bis(trifluoromethyl)benzoyl)piperidin-2-yl)-N-(4-chlorophenyl)acetamide

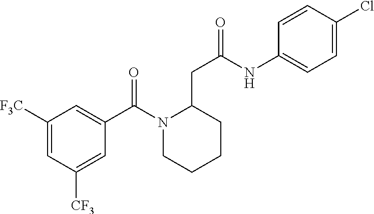

2-(1-(3,5-bis(trifluoromethyl)benzoyl)piperidin-2-yl)acetic acid (91 mg) was dissolved in anhydrous THF (1.0 mL) and to the solution was added 4-chloroaniline (33 mg), followed by 1-Hydroxybenzotriazole hydrate (38 mg), N-(3-Dimethylaminopropyl)-N′-ethyl-carbodiimide hydrochloride (55 mg), and N,N-Diisopropylethylamine (0.050 mL). The solution was allowed to stir at room temperature overnight under nitrogen. Ethyl acetate (10 mL) was added and the organic layer was washed with NaHCO₃ (15 mL), followed by IN HCl (15 mL) and brine (15 mL), dried (MgSO₄), and concentrated. The crude material was purified using column chromatography (CH₂Cl₂/Methanolic ammonia, 99/1 to 98.5/1.5) to afford 30 mg of the title compound as a yellow oil. ¹H NMR (500 MHz, CDCl₃) δ 8.90, 7.95, 7.80, 7.52, 7.25, 3.46, 3.29, 3.01, 2.74, 1.86-1.74, 1.55.

### Reaction 27 (Compound 27): 1-(3,5-bis(trifluoromethyl)benzoyl)-N-(4-chlorophenyl)pyrrolidine-3-carboxamide

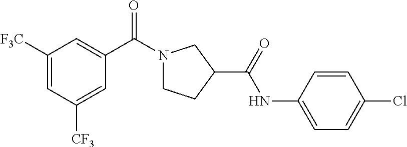

N-(4-chlorophenyl)pyrrolidine-3-carboxamide (50 mg) was dissolved in dichloromethane (1.5 mL) and to the solution was added 3,5-bis(trifluoromethyl)benzoyl chloride (0.029 mL) followed by triethylamine (0.046 mL). The solution was stirred overnight at room temperature. The solution was concentrated and partitioned between 2N HCl (3 mL) and EtOAc (2×3 mL). The organic layer was washed with brine (3 mL), dried (MgSO₄), and concentrated. The crude material was purified using column chromatography (Hexanes/EtOAc, 11/9) to afford 18 mg of the title compound as a clear oil. ¹H NMR (500 MHz, CDCl₃) δ 8.23, 8.08, 8.00, 7.96, 7.48, 7.42, 7.28-7.24, 4.10-3.48, 3.18, 3.09; MS (O) m/z 465 (M+).

### Reaction 28 (Compound 28): N¹-(3,5-bis(trifluoromethyl)benzyl)-N³-(4-chlorophenyl)propane-1,3-diamine

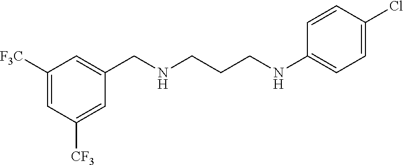

N-(3-(4-chlorophenylamino)-3-oxopropyl)-3,5-bis(trifluoromethyl)benzamide (50 mg) was dissolved in anhydrous THF (5.0 mL) and the solution was cooled to 0° C. To the cooled solution was added 1M Borane-tetrahydrofuran complex (0.400 mL), and the reaction was refluxed for 14 h. The mixture was then cooled to room temperature and quenched by addition of 1N HCl (15 mL). The reaction was refluxed for 1 h and cooled to 0° C. The solution was basified to pH 10 using 2M NaOH. The mixture was extracted with EtOAc (3×25 mL) and the organic layer was washed with brine (25 mL), dried (MgSO₄), and concentrated. The crude material was purified by column chromatography (CH₂Cl₂/Methanolic ammonia, 99/1 to 95/5) to afford 27 mg of the title compound. ¹H NMR (500 MHz, CDCl₃) δ 7.81, 7.78, 7.10, 6.50, 3.92, 3.18, 2.79, 1.78; MS (EI) m/z 411 (M+).

### Reaction 29 (Compound 29): N-(3-(5-chloroindolin-1-yl)-3-oxopropyl)-3,5-bis(trifluoromethyl)benzamide

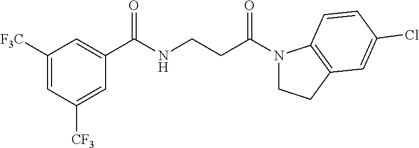

3-(3,5-bis(trifluoromethyl)benzamido)propanoic acid (329 mg) was dissolved in anhydrous THF (3.6 mL) and to the solution was added 5-chloroindoline (154 mg), followed by 1-Hydroxybenzotriazole hydrate (162 mg), N-(3-Dimethylaminopropyl)-N′-ethyl-carbodiimide hydrochloride (232 mg), and N,N-Diisopropylethylamine (0.200 mL). The solution was allowed to stir at room temperature overnight under nitrogen. Ethyl acetate (15 mL) was added and the organic layer was washed with NaHCO₃ (20 mL), followed by 1N HCl (20 mL) and brine (20 mL), dried (MgSO₄), and concentrated. The crude product was triturated with EtOAc to afford 34 mg of the title compound as a white solid. ¹H NMR (500 MHz, DMSO-d₆) δ 9.12, 8.51, 8.33, 8.07, 7.30, 7.21, 4.13, 3.62, 3.16, 2.81; MS (EI) m/z 487 (M+Na).

### Reaction 30 (Compound 30): N-(3-(4-chlorophenylamino)-3-oxopropyl)-3,5-bis(trifluoromethyl)benzamide

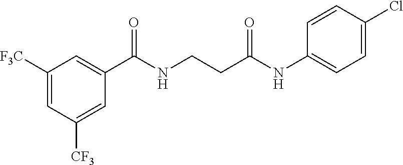

3-(3,5-bis(trifluoromethyl)benzamido)propanoic acid (330 mg) was dissolved in anhydrous THF (3.3 mL) and to the solution was added 4-chloroaniline (129 mg), followed by 1-Hydroxybenzotriazole hydrate (163 mg), N-(3-Dimethylaminopropyl)-N′-ethyl-carbodiimide hydrochloride (230 mg), and N,N-Diisopropylethylamine (0.210 mL). The solution was allowed to stir at room temperature overnight under nitrogen. Ethyl acetate (20 mL) was added and the organic layer was washed with NaHCO₃ (20 mL), followed by 1N HCl (20 mL) and brine (20 mL), dried (MgSO₄), and concentrated. The crude material was triturated with dichloromethane to afford 285 mg of the title compound as a white solid. ¹H NMR (500 MHz, DMSO-d₆) δ 10.13, 9.16, 8.49, 8.32, 7.62, 7.34, 3.60, 2.66; MS (EI) m/z 461 (M+Na).

### Reaction 31 (Compound 31): Benzyl 1-(4-chlorophenyl)-5-oxopyrrolidin-3-ylcarbamate

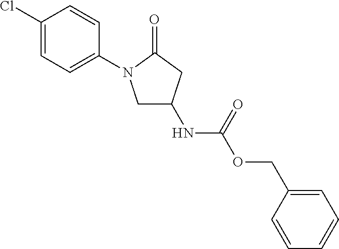 [0250] Prepared as described in J. Heterocyclic Chem. 1976, 13, 529-532.

### Reaction 32 (Compound 42)

### N-(3-((6-chloropyridin-3-yl)amino)propyl)-3,5-bis(trifluoromethyl)benzamide

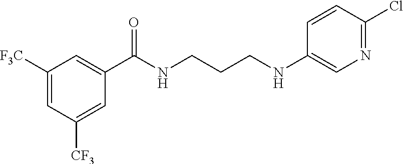

N¹-(6-chloropyridin-3-yl)propane-1,3-diamine was dissolved in THF (0.23 mL) and to the stirring solution was added 3,5-bis(trifluoromethyl)benzoic acid (20 mg) followed by 1-Hydroxybenzotriazole hydrate (11 mg), N-(3-Dimethylaminopropyl)-N′-ethyl-carbodiimide hydrochloride (16 mg), and N,N-Diisopropylethylamine (0.015 mL). The solution was allowed to stir at room temperature overnight under nitrogen. Ethyl acetate (5 mL) was added, and the organic layer was washed with sat. aq. NaHCO₃ (5 mL) followed by brine (5 mL), dried (MgSO₄), and concentrated. The crude material was purified using column chromatography (Hexanes/EtOAc, 1/1) to afford 18 mg of the title compound as a yellow crystalline solid.

¹H NMR (500 MHz, CDCl₃) δ 8.24, 8.00, 7.72, 7.08, 6.90, 3.64, 3.23, 1.95.

### Reaction 33 (Compound 43)

### N-(3-((4-chlorophenyl)amino)propyl)-3,5-difluorobenzamide

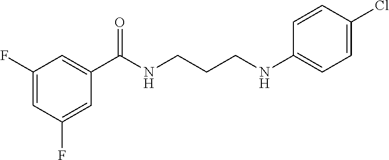

3,5-difluorobenzoic acid (47 mg) was dissolved in THF (0.5 mL), and to the stirring solution was added 1-Hydroxybenzotriazole hydrate (44 mg) followed by N-(3-Dimethylaminopropyl)-N′-ethyl-carbodiimide hydrochloride (62 mg). The mixture was allowed to stir for 20 minutes under nitrogen, and N,N-Diisopropylethylamine (0.142 mL) was added and stirring continued. The mixture was allowed to stir for 10 minutes, and N¹-(4-chlorophenyl)propane-1,3-diamine (50 mg) in THF (0.4 mL) was added. The solution was allowed to stir overnight at room temperature. Ethyl acetate (5 mL) was added and the organic layer was washed with sat. aq. NaHCO₃ (5 mL) followed by brine, dried (MgSO₄), filtered, and concentrated. The crude material was purified using column chromatography (Hexanes/EtOAc, 3/1) to afford 32 mg of the title compound as an off-white solid.

¹H NMR (500 MHz, CDCl₃) δ 7.24, 7.11, 6.93, 6.75, 6.54, 4.03, 3.55, 3.20, 1.89.

### Reaction 34 (Compound 44)

### N-(3-((5-chloropyridin-2-yl)amino)propyl)-3,5-bis(trifluoromethyl)benzamide

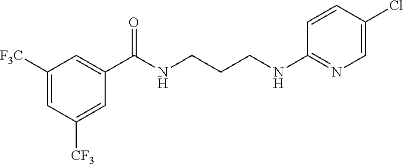

N¹-(5-chloropyridin-2-yl)propane-1,3-diamine (25 mg) was dissolved in THF (0.45 mL) and to the stirring solution was added 3,5-bis(trifluoromethyl)benzoic acid (38 mg) followed by 1-Hydroxybenzotriazole hydrate (22 mg), N-(3-Dimethylaminopropyl)-N′-ethyl-carbodiimide hydrochloride (31 mg), and N,N-Diisopropylethylamine (0.028 mL), and the reaction solution was allowed to stir at room temperature overnight. Ethyl acetate (5 mL) was added, and the organic layer was washed with sat. aq. NaHCO₃ (5 mL) followed by brine (5 mL), dried (MgSO₄) and concentrated. The crude material was purified using column chromatography (Hexanes/EtOAc, 1/1) to afford 33 mg of the title compound.

¹H NMR (500 MHz, CDCl₃) δ 8.36, 8.24, 8.04, 7.96, 7.37, 6.42, 4.82, 3.57, 1.86.

### Reaction 35 (Compound 45)

### 3,5-bis(trifluoromethyl)benzyl (1-(4-chlorophenyl)-5-oxopyrrolidin-3-yl)carbamate

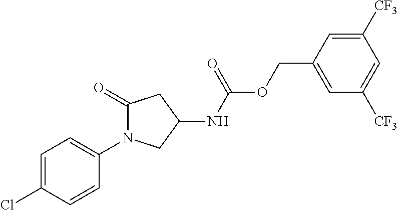

1-(4-chlorophenyl)-5-oxopyrrolidine-3-carboxylic acid (50 mg) was suspended in Toluene (3.5 mL) and to the mixture was added 3,5-bis(trifluoromethyl)benzyl alcohol (154 mg), followed by Triethylamine (0.044 mL), and Diphenylphosphoryl azide (0.054 mL). Reaction was heated at 130° C. for 6 hours, cooled to room temperature, and concentrated. The crude material was purified using column chromatography (Hexanes/EtOAc, 3/1 to 7/3 to 3/2) to afford 87 mg of compound. Purified compound was then triturated with chloroform to afford 49 mg of the title compound as an off-white solid.

¹H NMR (500 MHz, CDCl₃) δ 7.84, 7.80, 7.50, 7.28, 5.64, 5.21, 4.48, 4.14, 3.75, 2.98, 2.53.

### Reaction 36 (Compound 46)

### 3,5-difluorobenzyl (1-(4-chlorophenyl)-5-oxopyrrolidin-3-yl)carbamate

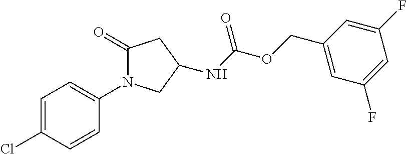

Title compound was prepared using the procedure described in Compound 45 using 3,5-difluorobenzyl alcohol (43 mL) to afford 39 mg of title compound as a clear oil.

¹H NMR (500 MHz, CDCl₃) δ 7.49, 7.28, 6.85, 6.75, 5.69, 5.07, 4.45, 4.11, 3.73, 2.96, 2.51.

### Reaction 37 (Compound 47)

### N-(1-(4-chlorophenyl)-5-oxopyrrolidin-3-yl)-3,5-bis(trifluoromethyl)benzamide

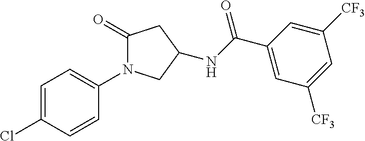

4-amino-1-(4-chlorophenyl)pyrrolidin-2-one (23 mg) was dissolved in Dichloromethane (0.218 mL). To the stirring solution was added 3,5-bis(trifluoromethyl)benzoyl chloride (0.022 mL) followed by Triethylamine (0.031 mL). The reaction mixture was stirred at room temperature overnight. The mixture was diluted with dichloromethane (10 mL) and was washed with 1M aq HCl (5 mL), followed by a wash with sat. aq. NaHCO₃ (5 mL) followed by brine (5 mL), dried (MgSO₄), and concentrated. The crude material was triturated with dichloromethane to afford 21 mg of title compound as a white solid.

¹H NMR (500 MHz, DMSO-d₆) δ 9.37, 8.52, 8.34, 7.74, 7.45, 4.72, 4.27-4.24, 3.83, 3.09-3.04, 2.64.

### Reaction 38 (Compound 48)

### N-(6-chloro-2-oxo-1,2,3,4-tetrahydroquinolin-4-yl)-3,5-bis(trifluoromethyl)benzamide

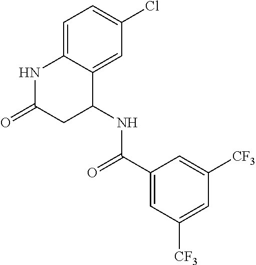

3-(3,5-bis(trifluoromethyl)benzamido)-3-(5-chloro-2-nitrophenyl)propanoic acid (17 mg) was heated with Iron(II) sulfate heptahydrate (98 mg) at 100° C. for 1 hour in 10% NH₄OH (0.700 mL) and Ethanol (0.700 mL). The reaction was cooled and additional ethanol was added. The mixture was filtered and extracted using ethyl acetate (3×5 mL). The organic layer was dried (MgSO₄) and concentrated to afford 7 mg of title compound as an off-white solid.

¹H NMR (500 MHz, DMSO-d₆) δ 10.45, 9.42, 8.55, 8.35, 7.36-7.32, 6.96, 5.39, 2.85-2.70.

### Reaction 39 (Compound 49)

### N-(1-(4-chlorophenyl)-5-oxopyrrolidin-3-yl)-3,5-difluorobenzamide

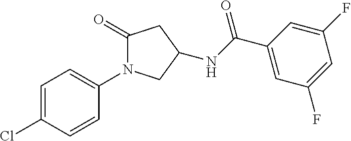

3,5-difluorobenzoic acid (14 mg) was dissolved in Dichloromethane (0.4 mL, and to the stifling solution was added 4-Dimethylaminopyridine (0.217 mg) followed by 4-amino-1-(4-chlorophenyl)pyrrolidin-2-one (15 mg) and N-(3-Dimethylaminopropyl)-N′-ethyl-carbodiimide hydrochloride (14 mg). The solution was stirred at room temperature overnight. Dichloromethane (2 mL) was added, and the solution was washed with 1N aq. HCl (2 mL) followed by sat. aq. NaHCO₃ (2 mL). The solvent was evaporated and the crude material was purified using column chromatography (Hexanes/EtOAc, 1/1) to afford 12 mg of title compound as a white solid.

¹H NMR (500 MHz, DMSO-d₆) δ 9.08, 7.73, 7.59, 7.51-7.43, 4.66-4.64, 4.24-4.21, 3.76, 3.04-2.99, 2.59; MS (EI) m/z 349 (M−1).

### Reaction 40 (Compound 50)

### N-((1-(3,5-bis(trifluoromethyl)benzyl)piperidin-3-yl)methyl)-4-chloroaniline

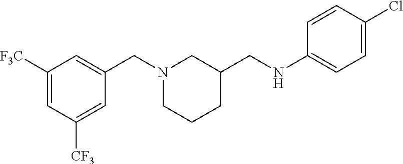

To a stirring solution of 1-(3,5-bis(trifluoromethyl)benzoyl)-N-(4-chlorophenyl)piperidine-3-carboxamide (50 mg) in anhydrous Tetrahydrofuran (2.1 mL) was added Lithium aluminum hydride (0.313 mL) and the reaction was stirred at 75° C. overnight. The reaction mixture was cooled to 0° C. and water (0.285 mL) was added drop wise, followed by a 15% NaOH solution (0.285 mL), followed by water (0.855 mL). The mixture was then filtered and washed with additional THF. The solution was concentrated and the crude material was purified using column chromatography (Hexanes/EtOAc, 85/15) to afford 14 mg of title compound as a yellow oil.

¹H NMR (500 MHz, CDCl₃) δ 7.79, 7.76, 7.10, 6.48, 3.76-3.52, 3.01, 2.82, 2.67, 2.08, 1.94, 1.83-1.59, 1.10; MS (EI) m/z 449 (M−1).

### Reaction 41 (Compound 51)

### N-(3,5-bis(trifluoromethyl)phenyl)-1-(4-chlorophenyl)-5-oxopyrrolidine-3-carboxamide

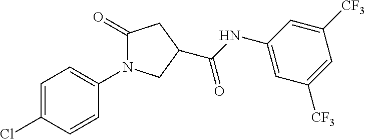

To a stirring solution of 1-(4-chlorophenyl)-5-oxopyrrolidine-3-carboxylic acid (50 mg) and 3,5-bis(trifluoromethyl)aniline (0.039 mL) in N,N-Dimethylformamide (0.417 mL) was added N-(3-Dimethylaminopropyl)-N′-ethyl-carbodiimide hydrochloride (48 mg). The reaction was stirred overnight at room temperature. The DMF was removed under high vacuum and water (10 mL) was then added. The aqueous layer was extracted with ethyl acetate (3×10 mL), and the organic layers were combined, washed with brine (10 mL), dried (MgSO₄), and concentrated. Crude material was triturated with dichloromethane and filtered to afford 45 mg of title compound as a white crystalline solid.

¹H NMR (500 MHz, CDCl₃) δ 8.42, 8.09, 7.64, 7.53, 7.33, 7.03, 4.26-4.22, 4.02, 3.40, 3.05-2.89; MS (EI) m/z 449 (M−1).

### Reaction 42 (Compound 52)

### N-(1-(4-chloro-3-methoxyphenyl)-5-oxopyrrolidin-3-yl)-3,5-bis(trifluoromethyl)benzamide

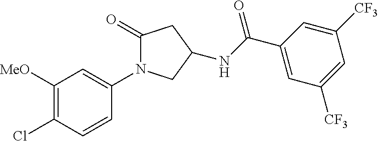

4-amino-1-(4-chloro-3-methoxyphenyl)pyrrolidin-2-one (12 mg) was dissolved in 2M aq. NaOH (0.100 mL), and to the stirring solution was added 3,5-bis(trifluoromethyl)benzoyl chloride (0.013 mL). The mixture was stirred overnight at room temperature. After stirring overnight, the reaction mixture was extracted with ethyl acetate (3×5 mL). The organic layers were combined, washed with 1M aq. HCl (5 mL) followed by brine (5 mL), dried (MgSO₄), and concentrated to afford 15 mg of title compound as a white solid.

¹H NMR (500 MHz, DMSO-d₆) δ 9.39, 8.53, 8.45, 8.35, 7.57, 7.43-7.40, 7.27-7.25, 4.73, 4.30-4.26, 3.85, 3.10-3.05, 2.65; MS (EI) m/z 479 (M−1).

### Reaction 43 (Compound 53)

### N-((1-(4-chlorophenyl)-2-oxopyrrolidin-3-yl)methyl)-3,5-bis(trifluoromethyl)benzamide

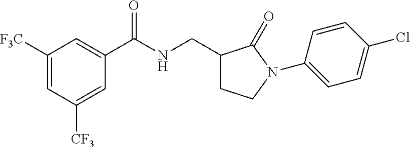

3-(aminomethyl)-1-(4-chlorophenyl)pyrrolidin-2-one (15 mg) was suspended in Dichloromethane (0.668 mL), and to the stirring mixture was added 3,5-bis(trifluoromethyl)benzoyl chloride (0.014 mL) followed by Triethylamine (0.011 mL). The reaction was allowed to stir at room temperature overnight. The mixture was concentrated, and the solid was then partitioned between 2M aq. HCl (5 mL) and ethyl acetate (5 mL). The aqueous layer was extracted an additional time with ethyl acetate (5 mL), and the organic layers were combined and washed with sat. aq. NaHCO₃ (5 mL) followed by brine (5 mL), dried (MgSO₄), filtered, and concentrated. The crude material was purified using column chromatography (Hexanes/EtOAc, 3/2 to 1/1) to afford 10 mg of title compound as a white crystalline solid.

¹H NMR (500 MHz, CDCl₃) δ 8.31, 8.03, 7.95, 7.58, 7.38, 4.21-4.16, 3.92-3.83, 3.55-3.50, 3.01-2.95, 2.50-2.44, 2.05-1.97; MS (EI) m/z 463 (M−1).

### Reaction 44 (Compound 54)

### N-((5-chloro-2-oxoindolin-3-ylidene)methyl)-3,5-bis(trifluoromethyl)benzamide

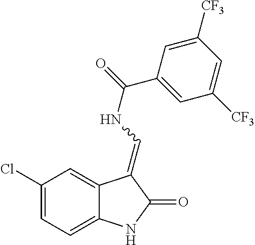

Anhydrous Pyridine (0.385 mL) was added to vial containing 3-(aminomethylene)-5-chloroindolin-2-one (54 mg), and the mixture was cooled to 0° C. 3,5-bis(trifluoromethyl)benzoyl chloride (0.275 mL) was added drop wise. The mixture was allowed to stir overnight at room temperature. The mixture was then poured into water (10 mL) and was extracted with dichloromethane (3×20 mL). The organic layer was then dried (MgSO₄), filtered, and concentrated. The crude material was triturated with dichloromethane to afford 87 mg of title compound as a bright yellow solid.

¹H NMR (500 MHz, DMSO-d₆) δ 12.33, 11.02, 8.69, 8.53, 8.49, 7.99, 7.22, 6.92.

### Reaction 45 (Compound 55)

### N-(1-(3-chloro-4-methoxyphenyl)-5-oxopyrrolidin-3-yl)-3,5-bis(trifluoromethyl)benzamide

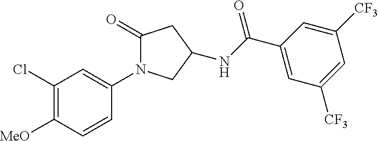

4-amino-1-(3-chloro-4-methoxyphenyl)pyrrolidin-2-one (16 mg) was dissolved in Dichloromethane (0.133 mL), and to the stirring solution was added 3,5-bis(trifluoromethyl)benzoyl chloride (0.014 mL) followed by Triethylamine (0.012 mL). The reaction was stirred overnight at room temperature. After stirring overnight, the reaction was concentrated to yield crude solid. The solid was then re-dissolved in ethyl acetate (5 mL) and was washed with sat. aq. NaHCO₃ (5 mL), followed by 1M aq. HCl (5 mL) then brine (5 mL). Organic layer was then dried (MgSO₄), filtered, and concentrated. The crude material was triturated with dichloromethane to afford 13 mg of title compound as a white solid.

¹H NMR (500 MHz, DMSO-d₆) δ 9.36, 8.52, 8.35, 7.90, 7.53, 7.17, 4.71, 4.70-4.25, 3.84, 3.81, 3.06-3.01, 2.61; MS (EI) m/z 479 (M−1).

### Reaction 46 (Compound 56)

### (3,5-bis(trifluoromethyl)phenyl)(3-(((4(4-chlorophenyl)amino)methyl)piperidin-1-yl)methanone

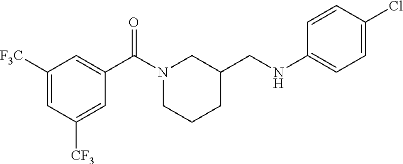

4-Chloroaniline (4 mg) and 1-(3,5-bis(trifluoromethyl)benzoyl)piperidine-3-carbaldehyde (17 mg) in DCE (0.105 mL) were cooled to 0° C. and were treated with Sodium triacetoxyborohydride (13 mg). The reaction was stirred overnight at room temperature. The mixture was then diluted with ethyl acetate (4 mL) and quenched with water (1 mL). The pH of the aqueous layer was adjusted to 10 using sat. aq. NaHCO₃ and 2M aq. NaOH. The organic layer was removed and was dried (MgSO₄), filtered, and concentrated. The crude material was purified using column chromatography (Hexanes/EtOAc, 4/1 to 7/3) and concentrated to afford 12 mg of title compound as a clear oil.

¹H NMR (500 MHz, CDCl₃) δ 7.94, 7.85, 7.14-7.08, 6.56, 6.39, 4.46-4.44, 4.10-3.99, 3.67-3.51, 3.17-2.94, 2.03-1.25; MS (O) m/z 465 (M+1)

### Reaction 47 (Compound 58)

### N-(1-(4-chlorophenyl)pyrrolidin-3-yl)-3,5-bis(trifluoromethyl)benzamide

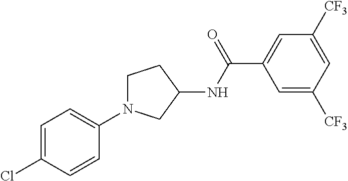

1-(4-chlorophenyl)pyrrolidin-3-amine (12 mg) was dissolved in Dichloromethane (0.610 mL), and to the stirring solution under nitrogen was added 3,5-bis(trifluoromethyl)benzoyl chloride (12 mL) followed by Triethylamine (0.026 mL). The reaction was allowed to stir overnight at room temperature under nitrogen. The reaction mixture was concentrated and the solid was partitioned between sat. aq. NaHCO₃ (5 mL) and ethyl acetate (10 mL). The aqueous layer was extracted a second time with ethyl acetate (10 mL), and the organics were combined, washed with brine (10 mL), dried (MgSO₄), filtered, and concentrated. The crude material was triturated with dichloromethane to afford 5.1 mg of title compound as a white solid.

¹H NMR (500 MHz, CDCl₃) δ 9.14, 8.52, 8.33, 7.19, 6.57, 4.67, 3.61-3.58, 3.45-3.41, 3.27-3.24; MS (EI) m/z 435 (M−1).

### Example 2

### Dual Luciferase and WST-1 Assays

### Materials and Methods

Dual Luciferase Assay: Seed PC-3 prostate cancer cells (40,000 cells/well) were grown in 96-well plates in 10% FBS containing DMEM medium. Cells were transiently transfected with the Gα12QL activator of the Rho/MKL1 pathway, along with the SRE.L-firefly luciferase reporter construct for 6 hours. Additionally, cells were co-transfected with the TK-Renilla luciferase reporter as an indicator of non-specific compound effects. Various concentrations of selected compounds were added to the 96-well plates. Plates were incubated for 19 hours at 37° C. and 5% CO₂ in 0.5% FBS containing DMEM medium. Cells were lysed with 1X Passive Lysis Buffer (Promega). Plates were incubated for 30 minutes at room temperature. Luminescence counts were read with a Victor2 (Perkin-Elmer) plate reader.

WST1 Cell Viability Assay: One hour prior to cell lysis for the dual luciferase assay, 10 μl per well of WST1 reagent (Roche) was added to the 96-well plates. Plates were incubated for 1 hour at 37° C. and 5% CO₂. Cell viability was measured by WST1 absorbance at 450 nm with a Victor2 (Perkin-Elmer) plate reader.

Results

Compounds were tested as described in Materials and Methods. Synthesis schemes are shown in Example 1. Table 1 shows the results of the dual luciferase assays and the viability (WST1) assay.

TABLE 1
Summary of SRE.L-Luciferase Reporter Results.

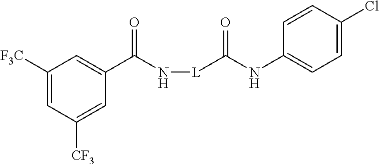

| | | IC₅₀ | % inh | % inh | % inh | |
|---|---|---|---|---|---|---|
| Compound | | SRE.L | SRE.L | PRL-TK | WST-1 | Scheme |
| No. | L | (μM)^{a} | (10, 100 μM)^{b} | (10, 100 μM)^{b} | (10, 100 μM)^{b} | No. |
| CCG-1423 | —OCH(CH₃)— | 1.5 | 74, ND | 48, ND | 44, ND | |
| 1 | —OCH₂— | 4.7 | 71, 100 | 53, 89 | 42, 91 | 3 |
| 30 | —CH₂CH₂— | 25 | 44, 66 | 0, 15 | 0, 6 | 3 |
| 3 | —CH₂— | 33 | 45, 85 | 15, 25 | 0, 30 | 3 |
| 2 | —CH₂CH₂CH₂— | 21 | 37, 79 | 5, 42 | 0, 12 | 3 |
| 32 | —CH₂CH₂CH₂CH₂— | NA | 0, 12 | 0, 10 | 0, 0 | 3 |

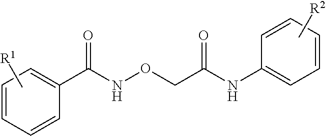

| | | | IC₅₀ | % inh | % inh | % inh | |
|---|---|---|---|---|---|---|---|
| Compound | | | SRE.L | SRE.L | PRL-TK | WST-1 | Scheme |
| No. | R¹ | R² | (μM)^{a} | (10, 100 μM)^{b} | (10, 100 μM)^{b} | (10, 100 μM)^{b} | No. |
| 1 | 3,5-bis(CF₃) | 4-Cl | 4.7 | 71, 100 | 53, 89 | 42, 91 | 3 |
| 10 | 3,5-bis(CF₃) | 3-Cl | 5.9 | 65, 100 | 51, 89 | 49, 97 | 3 |
| 33 | 3,5-bis(CF₃) | 4-H | 36 | 13, 65 | 33, 59 | 0, 37 | 3 |
| 34 | 3-CF₃ | 4-Cl | 27 | 25, 86 | 5, 19 | 0, 58 | 3 |
| 35 | 4-CF₃ | 4-Cl | 29 | 26, 91 | 6, 0 | 0, 56 | 3 |
| 36 | 4-H | 4-Cl | NA | NA | NA | NA | 3 |
| 6 | 4-Cl | 3,5-bis(CF₃) | 8.6 | 58, 100 | 19, 87 | 11, 96 | |

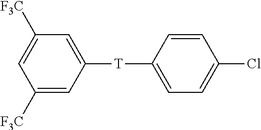

| | | IC₅₀ | % inh | % inh | % inh | |
|---|---|---|---|---|---|---|
| Compound | | SRE.L | SRE.L | PRL-TK | WST-1 | Scheme |
| No. | T | (μM)^{a} | (10, 100 μM)^{b} | (10, 100 μM)^{b} | (10, 100 μM)^{b} | No. |
| 30 | —CONHCH₂CH₂NHCO— | 38 | 38, 64 | 0, 22 | 0, 10 | 3 |
| 37 | —CONHCH₂CH₂N(Me)CO— | >1000 | 20, 25 | 0, 10 | 0, 0 | 3 |
| 15 | —CONHCH₂CH₂CH₂NH— | 5.1 | 70, 80 | 37, 35 | 0, 11 | 1 |
| 22 | —CH₂NHCH₂CH₂CONH— | 8.1 | 64, 100 | 12, 91 | 0, 92 | 2 |
| 28 | —CH₂NHCH₂CH₂CH₂NH— | 9.1 | 65, 100 | 6, 90 | 0, 89 | 9 |
| 19 | —CONHCH₂CH₂CH₂O— | 8.9 | 55, 86 | 28, 65 | 0, 38 | 5 |

| | | | % inh | % inh | % inh | |
|---|---|---|---|---|---|---|
| Com- | | IC₅₀ | SRE.L | PRL-TK | WST-1 | |
| pound | | SRE.L | (10, 100 | (10, 100 | (10, 100 | Scheme |
| No. | Structure | (μM)^{a} | μM)^{b} | μM)^{b} | μM)^{b} | No. |
| 30 | 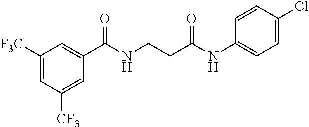 | 38 | 38, 64 | 0, 22 | 0, 10 | 3 |
| 4 | 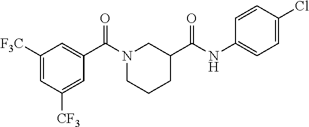 | 10 | 37, 78 | 19, 34 | 0, 14 | 3 |
| 5 | 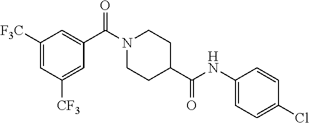 | 16 | 17, 87 | 0, 17 | 0, 39 | 3 |
| 7 | 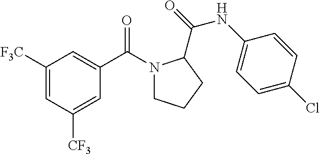 | 69 | 23, 83 | 12, 27 | 0, 22 | 3 |
| 16 | 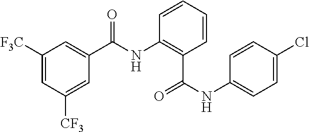 | 18 | 34, ND | 0, ND | 0, ND | 3 |
| 14 | 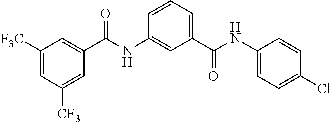 | 1.7 | 79, ND | 0, ND | 38, ND | 3 |
| 38 | 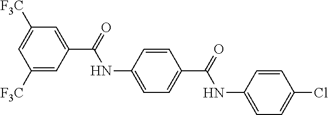 | NA | 0, ND | 0, ND | 0, ND | 3 |
| 39 | 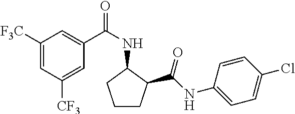 | NA | | | | 3 |
| 29 | 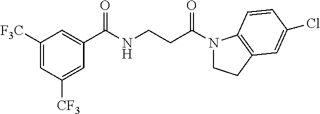 | 13 | 15, 40 | 0, 1 | 0, 0 | 4 |
| 24 | 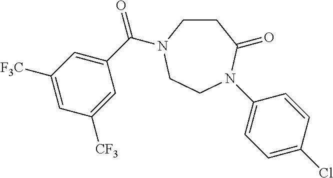 | NA | | | | 6 |
| 26 | 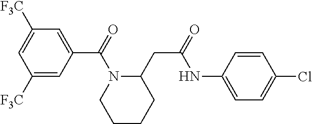 | 9.9 | 41, 85 | 0, 76 | 0, 27 | 3 |
| 59 | 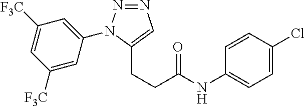 | NA | −3.0, 4.7 | −31.8, −13.3 | −9.5, −11.8 | |
| 18 | 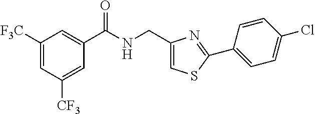 | 4.1 | 50, 45 | 39, 38 | 0, 0 | 5 |
| 23 | 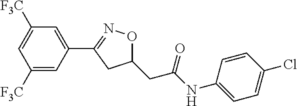 | 4.6 | 77, 87 | 58, 73 | 0, 66 | 7 |
| 40 | 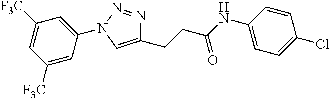 | NA | | | | |
| 41 | 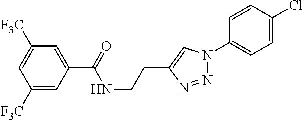 | NA | | | | |
| 27 | 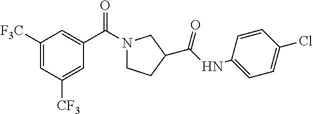 | 15.6 | 30, 100 | 14, 92 | 0, 86 | 3 |
| 9 | 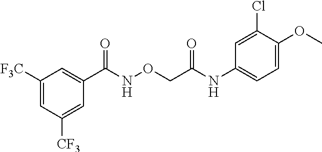 | 11.4 | | | | 3 |
| 11 | 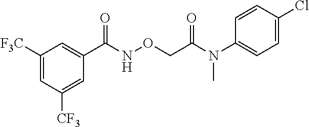 | 111 | | | | 3 |
| 12 | 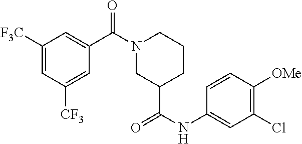 | 6 | | | | 3 |
| 13 | 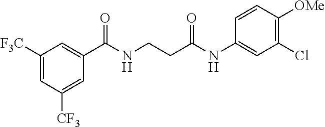 | 142 | | | | 3 |
| 17 | 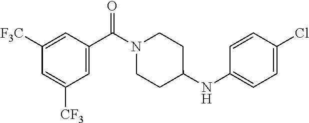 | 10.8 | | | | 8 |
| 20 | 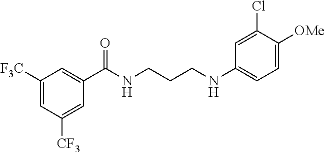 | 9.9 | | | | 1 |
| 21 | 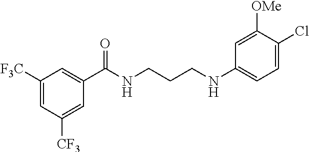 | 7.3 | | | | 1 |
| 24 | 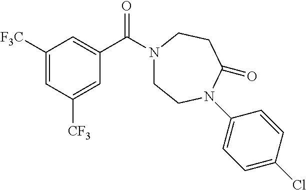 | 30% inhi- bition at 100 μm | | | | 6 |
| 25 | 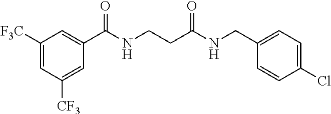 | 5 | | | | 3 |
| 31 | 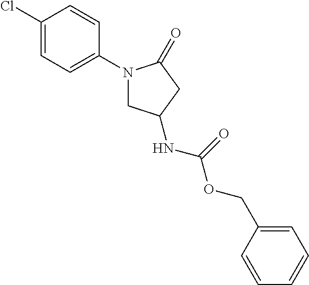 | 54.7 | | | | |
| 42 | 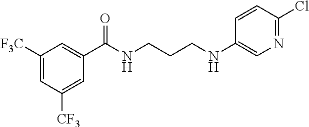 | 24.4 | −1.7, 95.1 | ND, ND | −7.4, 64.7 | Reaction 32 |
| 43 | 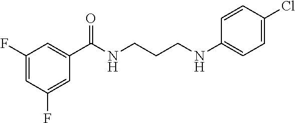 | 34.7 | −6.5, 95.7 | ND, ND | −9.9, 73.0 | Reaction 33 |
| 44 | 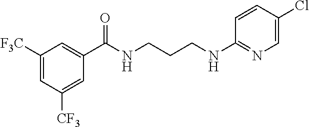 | 654.0 | 20.5, 82.9 | ND, ND | 17.6, 39.4 | Reaction 34 |
| 45 | 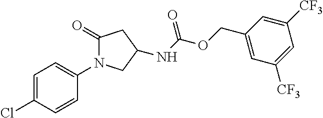 | 13.4 | 9.4, 68.0 | ND, ND | 0.6, 8.4 | Reaction 35 |
| 46 | 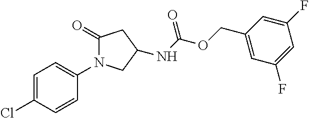 | ND | 13.7, 76.1 | ND, ND | 0.1, 67.5 | Reaction 36 |
| 47 | 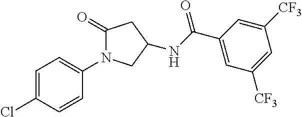 | 18.0 | 18.0, 57.8 | ND, ND | −0.8, 14.3 | Reaction 37 |
| 48 | 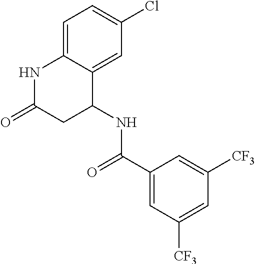 | ND | 4.9, 2.5 | ND, ND | 4.3, −2.7 | Reaction 38 |
| 49 | 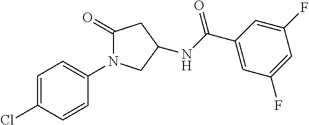 | ND | −8.4, 13.4 | ND, ND | −6.1, 0.6 | Reaction 39 |
| 50 | 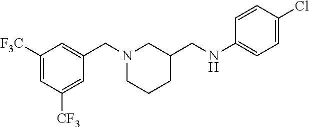 | ND | 0.9, 70.2 | ND, ND | 20.9, 32.2 | Reaction 40 |
| 51 | 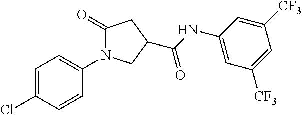 | ND | 74.3, 99.2 | ND, ND | 55.9, 74.6 | Reaction 41 |
| 52 | 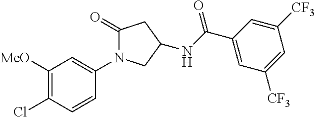 | ND | −18.4, 16.9 | ND, ND | −0.3, 42.2 | Reaction 42 |
| 53 | 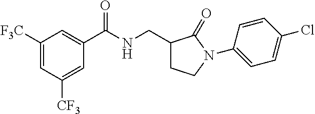 | ND | 14.1, 32.3 | ND, ND | 7.9, 26.9 | Reaction 43 |
| 54 | 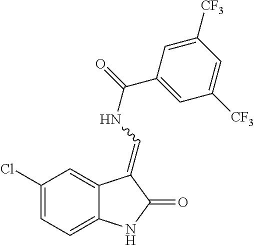 | ND | 6.5, 12.2 | ND, ND | 10.6, 17.7 | Reaction 44 |
| 55 | 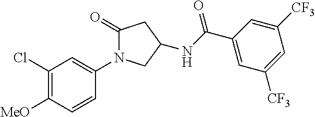 | ND | 4.5, 26.0 | ND, ND | 7.4, 23.9 | Reaction 45 |
| 56 | 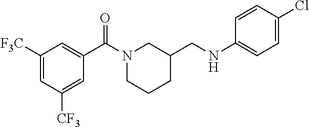 | ND | 11.5, 8.3 | ND, ND | 24.8, 22.4 | Reaction 46 |
| 57 | 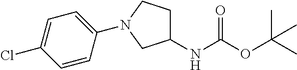 | ND | −44.4, 18.7 | ND, ND | 9.2, 21.1 | Prepara- tion on 37 |
| 58 | 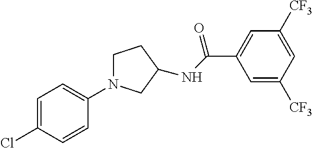 | ND | −4.6, 10.5 | ND, ND | −10.5, −1.7 | Reaction 47 |

Biological activity of the synthetic chemical analogs of CCG-1423 were assessed in the SRE.L luciferase reporter assay. PC-3 prostate cancer cells were co-transfected with 2 ng of the Gα₁₂Q231L expression plasmid along with 50 ng of the SRE.L and 7 ng of the pRL-TK luciferase reporter plasmids as described in Materials and Methods supra. Cells were treated with 0 (vehicle (DMSO) alone), 1, 3, 10, 30, and 100 μM of compound for 19 hrs after transfection before lysis. Luminescence was determined as described in the Materials and Methods supra. For some compounds, the concentrations of 30 and 100 μM were not tested. Just before cell lysis, the viability of the cells was measured using the WST-1 cell proliferation reagent as described in Materials and Methods supra. Data are expressed as percentage of inhibition (DMSO alone = 0%). The experiments were performed three separate times to achieve n = 3 in triplicate. NA, no activity (defined as IC₅₀ SRE.L value exceeding 100 μM, or no inhibitory activity seen at highest level tested; such compounds may have activity applying a less stringent standard); ND, no data

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in molecular biology, cancer biology, genetics, or related fields are intended to be within the scope of the following claims.

1. A composition comprising a compound selected from the group consisting of 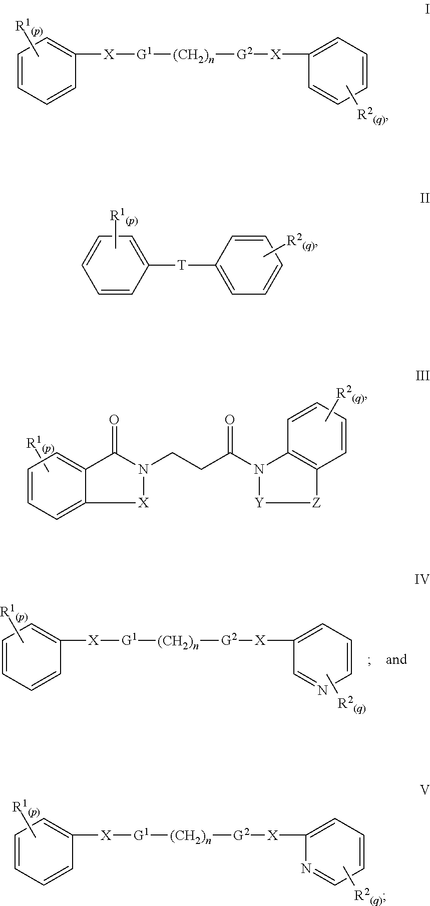
wherein G¹ and G² may be independently selected from the group consisting of (C═O)NH, NH(C═O), (C═O)NHO, NH, O, NH(C═O)O, O(C═O)NH, and heteroaryl;
wherein p and q may be independently 1, 2, 3, 4, or 5;
wherein R¹ and R² may be one or more functional groups independently selected from the group consisting of halogen, CF₃, OCF₃, CN, O(C1-C6 alkyl), and C1-C6 alkyl;
wherein X, Y, Z are independently: (CH₂)ₘ wherein m is 0, 1, or 2 and n is 1, 2, or 3;
wherein G¹ and G² are not CONH when R¹ is 3,5-bis(CF₃) and R² is 4-Cl;
wherein heteroaryl is selected from the group consisting of 1,3-thiazole and isoxazole; and
wherein T is selected from the group consisting of:

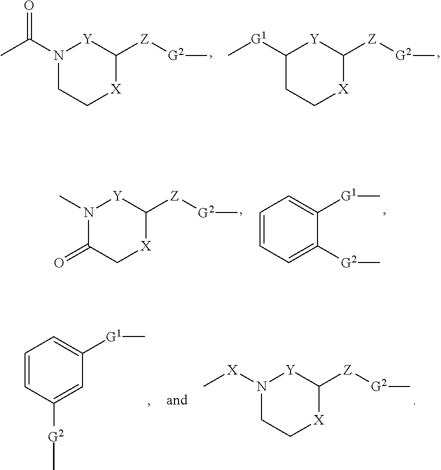

2. The composition of claim 1, wherein said composition is selected from the group consisting of 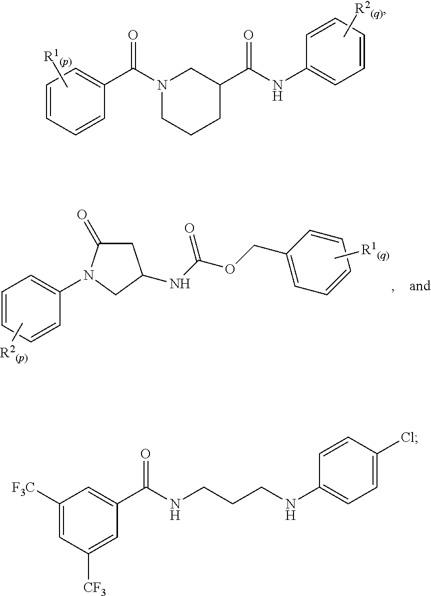
wherein p and q may be independently 1, 2, 3, 4, or 5.

3. The composition of claim 1 wherein said composition is in a pharmaceutically appropriate formulation for administration to a human subject.

4. The composition of claim 2, wherein said composition is in a pharmaceutically appropriate formulation for administration to a human subject.

5. The composition of claim 1, wherein said composition has an IC₅₀ value for rho protein of between 1 and 50,000 nM.

6. The composition of claim 5, wherein said rho protein is selected from the group consisting of rhoA and rhoC.

7. The composition of claim 1, wherein said composition results in 50% or less inhibition of WST-1 metabolism in an in vitro cell when said composition is administered to said in vitro cell at a concentration of 10 μM.

8. A method of treating a rho-mediated disease in a subject comprising administering a compound of claim 1 in a pharmaceutically appropriate formulation to said subject.

9. A method of preventing a rho-mediated disease in a subject comprising administering a compound of claim 1 in a pharmaceutically appropriate formulation to said subject.

10. The method of claim 8, wherein said rho-mediated disease is selected from the group consisting of cancer, inflammation, inflammatory disease, pulmonary arterial hypertension, axon regeneration following nerve damage, Raynaud's phenomenon, cerebral vascular disease, cardiovascular disease, and erectile dysfunction.

11. The method of claim 10, wherein said cancer type is selected from the group consisting of fibrosarcoma, myxosarcoma, liposarcoma, chondrosarcoma, osteogenic sarcoma, chordoma, angiosarcoma, endotheliosarcoma, lymphangiosarcoma, Ewing's tumor, lymphangioendotheliosarcoma, synovioma, mesothelioma, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, polycythemia vera, lymphoma, multiple myeloma, Waldenstrom's macroglobulinemia, and heavy chain disease.

12. The method of claim 10, wherein said inflammatory disease is selected from the group consisting of arthritis, rheumatoid arthritis, psoriatic arthritis, osteoarthritis, degenerative arthritis, polymyalgia rheumatic, ankylosing spondylitis, reactive arthritis, gout, pseudogout, inflammatory joint disease, systemic lupus erythematosus, polymyositis, fibromyalgis, achilles tendinitis, achondroplasia, acromegalic arthropathy, adhesive capsulitis, adult onset Still's disease, anserine bursitis, avascular necrosis, Behcet's syndrome, bicipital tendinitis, Blount's disease, brucellar spondylitis, bursitis, calcaneal bursitis, calcium pyrophosphate deposition disease, crystal deposition disease, Caplan's syndrome, carpal tunnel syndrome, chondrocalcinosis, chondromalacia patellae, chronic synovitis, chronic recurrent multifocal osteomyelitis, Churg-Strauss syndrome, Cogan's syndrome, corticosteroid-induced osteoporosis, costosternal syndrome, CREST syndrome, cryoglobulinemia, degenerative joint disease, dermatomyositis, diabetic finger sclerosis, diffuse idiopathic skeletal hyperostosis, discitis, discoid lupus erythematosus, drug-induced lupus, Duchenne's muscular dystrophy, Dupuytren's contracture, Ehlers-Danlos syndrome, enteropathic arthritis, epicondylitis, erosive inflammatory osteoarthritis, exercise-induced compartment syndrome, Fabry's disease, familial Mediterranean fever, Farber's lipogranulomatosis, Felty's syndrome, Fifth's disease, flat feet, foreign body synovitis, Freiberg's disease, fungal arthritis, Gaucher's disease, giant cell arteritis, gonococcal arthritis, Goodpasture's syndrome, granulomatous arteritis, hemarthrosis, hemochromatosis, Henoch-Schonlein purpura, Hepatitis B surface antigen disease, hip dysplasia, Hurler syndrome, hypermobility syndrome, hypersensitivity vasculitis, hypertrophic osteoarthropathy, immune complex disease, impingement syndrome, Jaccoud's arthropathy, juvenile ankylosing spondylitis, juvenile dermatomyositis, juvenile rheumatoid arthritis, Kawasaki disease, Kienbock's disease, Legg-Calve-Perthes disease, Lesch-Nyhan syndrome, linear scleroderma, lipoid dermatoarthritis, Lofgren's syndrome, Lyme disease, malignant synovioma, Marfan's syndrome, medial plica syndrome, metastatic carcinomatous arthritis, mixed connective tissue disease, mixed cryoglobulinemia, mucopolysaccharidosis, multicentric reticulohistiocytosis, multiple epiphyseal dysplasia, mycoplasmal arthritis, myofascial pain syndrome, neonatal lupus, neuropathic arthropathy, nodular panniculitis, ochronosis, olecranon bursitis, Osgood-Schlatter's disease, osteoarthritis, osteochondromatosis, osteogenesis imperfecta, osteomalacia, osteomyelitis, osteonecrosis, osteoporosis, overlap syndrome, pachydermoperiostosis Paget's disease of bone, palindromic rheumatism, patellofemoral pain syndrome, Pellegrini-Stieda syndrome, pigmented villonodular synovitis, piriformis syndrome, plantar fasciitis, polyarteritis nodos, Polymyalgia rheumatic, polymyositis, popliteal cysts, posterior tibial tendinitis, Pott's disease, prepatellar bursitis, prosthetic joint infection, pseudoxanthoma elasticum, psoriatic arthritis, Raynaud's phenomenon, reactive arthritis/Reiter's syndrome, reflex sympathetic dystrophy syndrome, relapsing polychondritis, retrocalcaneal bursitis, rheumatic fever, rheumatoid vasculitis, rotator cuff tendinitis, sacroiliitis, salmonella osteomyelitis, sarcoidosis, saturnine gout, Scheuermann's osteochondritis, scleroderma, septic arthritis, seronegative arthritis, shigella arthritis, shoulder-hand syndrome, sickle cell arthropathy, Sjogren's syndrome, slipped capital femoral epiphysis, spinal stenosis, spondylolysis, staphylococcus arthritis, Stickler syndrome, subacute cutaneous lupus, Sweet's syndrome, Sydenham's chorea, syphilitic arthritis, systemic lupus erythematosus, Takayasu's arteritis, tarsal tunnel syndrome, tennis elbow, Tietse's syndrome, transient osteoporosis, traumatic arthritis, trochanteric bursitis, tuberculosis arthritis, arthritis of Ulcerative colitis, undifferentiated connective tissue syndrome, urticarial vasculitis, viral arthritis, Wegener's granulomatosis, Whipple's disease, Wilson's disease, and yersinial arthritis.

13. The method of claim 9, further comprising administering an agent selected from the group consisting of a chemotherapeutic agent or an anti-inflammatory agent.

14. A method of reducing metastatic spread of a cancer cell in a subject comprising administering a compound of claim 1 in a pharmaceutically appropriate formulation to said subject.

15. A method of reducing growth of a cancer cell in a subject comprising administering a compound of claim 1 in a pharmaceutically appropriate formulation to said subject.

16. A method of reducing growth of a tumor in a subject comprising administering a compound of claim 1 in a pharmaceutically appropriate formulation to said subject.

17. A method of inhibiting the in vitro activity of rho protein comprising exposing said rho protein to a composition of claim 1.

18. The method of claim 17, wherein said rho protein is selected from the group consisting o rhoA and rhoC.

19. The method of claim 17, wherein said activity is assessed by measuring the expression of a rho-mediated gene.

20. The method of claim 19, wherein said measurement comprises assessing the level of a rho-mediated gene transcript.

21. The method of claim 19, wherein said measurement comprises assessing the level of a rho-mediated protein.

22. The method of claim 19, wherein said measurement comprises assessing the level of activity of a rho-mediated protein.

23. The composition of claim 1, comprising compound II, wherein T is: 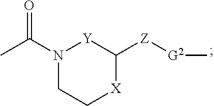
wherein X is (CH₂)₁, Y is (CH₂)₁, and Z is (CH₂)₀.

24. The composition of claim 23, wherein R₁ is a halogen and R₂ is a heterocyclic subgroup.

25. The composition of claim 24, wherein R₂ is a furan.

26. The composition of claim 23, wherein G₂ is (C═O)NH.

27. The composition of claim 1, comprising compound II, wherein T is: 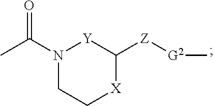
wherein R₁ is a halogen and R₂ is a heterocyclic subgroup, and wherein said heterocyclic subgroup is a furan;
wherein X is (CH₂)₁, Y is (CH₂)₁, and Z is (CH₂)₀; and
wherein G₂ is (C═O)NH.
